SEQUENCE LISTING (33 sequences)

SEQ ID NO: 1 - protein, 13 aa, Artificial Sequence
  Synthetic Construct
KASGGTFSSYAIS

SEQ ID NO: 2 - protein, 17 aa, Artificial Sequence
  Synthetic Construct
GIIPIFGTANYAQKFQG

SEQ ID NO: 3 - protein, 19 aa, Artificial Sequence
  Synthetic Construct
AREEGPYCSSTSCYGAFDI

SEQ ID NO: 4 - protein, 11 aa, Artificial Sequence
  Synthetic Construct
QGDSLRSYFAS

SEQ ID NO: 5 - protein, 8 aa, Artificial Sequence
  Synthetic Construct
YARNDRPA

SEQ ID NO: 6 - protein, 11 aa, Artificial Sequence
  Synthetic Construct
AAWDDSLNGYL

SEQ ID NO: 7 - protein, 117 aa, Artificial Sequence
  Synthetic Construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTV

SEQ ID NO: 8 - protein, 126 aa, Artificial Sequence
  Synthetic Construct
EVQLVQSGAEVKKPGSSVKVSCKASGGTFSSYAISWVRQAPGQGLEWMGGIIPIFGTANYAQKFQGRVTITADESTSTAYMELSSLRSEDTAVYYCAREEGPYCSSTSCYGAFDIWGQGTLVTVSS

SEQ ID NO: 9 - protein, 109 aa, Artificial Sequence
  Synthetic Construct
LLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVA

SEQ ID NO: 10 - protein, 101 aa, Artificial Sequence
  Synthetic Construct
QSVLTQDPAVSVALGQTVKITCQGDSLRSYFASWYQQKPGQAPTLVMYGVPDRFSGSKSGTSASLAISGLQSEDEADYYCAAWDDSLNGYLFGAGTKLTVL

SEQ ID NO: 11 - protein, 242 aa, Artificial Sequence
  Synthetic Construct
EVQLVQSGAEVKKPGSSVKVSCKASGGTFSSYAISWVRQAPGQGLEWMGGIIPIFGTANYAQKFQGRVTITADESTSTAYMELSSLRSEDTAVYYCAREEGPYCSSTSCYGAFDIWGQGTLVTVSSGGGGSGGGGSGGGGSQSVLTQDPAVSVALGQTVKITCQGDSLRSYFASWYQQKPGQAPTLVMYGVPDRFSGSKSGTSASLAISGLQSEDEADYYCAAWDDSLNGYLFGAGTKLTVL

SEQ ID NO: 12 - protein, 242 aa, Artificial Sequence
  Synthetic Construct
QSVLTQDPAVSVALGQTVKITCQGDSLRSYFASWYQQKPGQAPTLVMYGVPDRFSGSKSGTSASLAISGLQSEDEADYYCAAWDDSLNGYLFGAGTKLTVLGGGGSGGGGSGGGGSEVQLVQSGAEVKKPGSSVKVSCKASGGTFSSYAISWVRQAPGQGLEWMGGIIPIFGTANYAQKFQGRVTITADESTSTAYMELSSLRSEDTAVYYCAREEGPYCSSTSCYGAFDIWGQGTLVTVSS

SEQ ID NO: 13 - protein, 5 aa, Artificial Sequence
  Synthetic Construct
NYGMN

SEQ ID NO: 14 - protein, 5 aa, Artificial Sequence
  Synthetic Construct
GYAMS

SEQ ID NO: 15 - protein, 5 aa, Artificial Sequence
  Synthetic Construct
  MISC_FEATURE (1)..(1): Xaa is Arg or Gly
  MISC_FEATURE (3)..(3): Xaa is Ala or Gly
XYXMS

SEQ ID NO: 16 - protein, 17 aa, Artificial Sequence
  Synthetic Construct
  MISC_FEATURE (12)..(12): Xaa is Ala or Val
  MISC_FEATURE (13)..(13): Xaa is Gly or Asp
WINTYTGEPTYXXDFKG

SEQ ID NO: 17 - protein, 17 aa, Artificial Sequence
  Synthetic Construct
TISSGGTYIYYPDSVKG

SEQ ID NO: 18 - protein, 9 aa, Artificial Sequence
  Synthetic Construct
  MISC_FEATURE (1)..(1): Xaa is Ser or Thr
  MISC_FEATURE (3)..(3): Xaa is Thr or Asp
  MISC_FEATURE (4)..(4): Xaa is Thr or Ala
  MISC_FEATURE (5)..(6): Xaa is any amino acid
  MISC_FEATURE (7)..(7): Xaa is Tyr or Phe
XGXXXXXYA

SEQ ID NO: 19 - protein, 9 aa, Artificial Sequence
  Synthetic Construct
TGTTAILNG

SEQ ID NO: 20 - protein, 9 aa, Artificial Sequence
  Synthetic Construct
SGDGYWYYA

SEQ ID NO: 21 - protein, 10 aa, Artificial Sequence
  Synthetic Construct
  MISC_FEATURE (5)..(6): Xaa is any amino acid
  MISC_FEATURE (10)..(10): Xaa is Gly or Ala
DNYGXXYDYX

SEQ ID NO: 22 - protein, 10 aa, Artificial Sequence
  Synthetic Construct
  MISC_FEATURE (6)..(6): Xaa is Val or Ile
  MISC_FEATURE (9)..(9): Xaa is Met or Ile
  MISC_FEATURE (10)..(10): Xaa is His or Tyr
SASSSXSYXX

SEQ ID NO: 23 - protein, 15 aa, Artificial Sequence
  Synthetic Construct
RASKSVSTSGYSYMH

SEQ ID NO: 24 - protein, 7 aa, Artificial Sequence
  Synthetic Construct
  MISC_FEATURE (1)..(1): Xaa is Ser or Gly
XTSNLAS

SEQ ID NO: 25 - protein, 7 aa, Artificial Sequence
  Synthetic Construct
LASNLES

SEQ ID NO: 26 - protein, 9 aa, Artificial Sequence
  Synthetic Construct
  MISC_FEATURE (5)..(5): Xaa is Ser or Asn
  MISC_FEATURE (8)..(8): Xaa is Ser or Phe
QQRSXYPXT

SEQ ID NO: 27 - protein, 9 aa, Artificial Sequence
  Synthetic Construct
QHSRELPFT

SEQ ID NO: 28 - protein, 116 aa, Artificial Sequence
  Synthetic Construct
VQLQESGGGLVQPGGSMKLSCVASGFTFSNYWMNWVRQSPEKGLEWVAEIRLKSNNYATHYAESVKGRFTISRDDSKSSVYLQMNNLRAEDTGIYYCTGVGQFAYWGQGTTVTVSS

SEQ ID NO: 29 - protein, 106 aa, Artificial Sequence
  Synthetic Construct
DIELTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKL

SEQ ID NO: 30 - protein, 106 aa, Artificial Sequence; identical to SEQ ID NO: 29
  Synthetic Construct

SEQ ID NO: 31 - protein, 121 aa, Artificial Sequence
  Synthetic Construct
GGGGSVQLQESGGGLVQPGGSMKLSCVASGFTFSNYWMNWVRQSPEKGLEWVAEIRLKSNNYATHYAESVKGRFTISRDDSKSSVYLQMNNLRAEDTGIYYCTGVGQFAYWGQGTTVTVSS

SEQ ID NO: 32 - protein, 237 aa, Artificial Sequence
  Synthetic Construct
VQLQESGGGLVQPGGSMKLSCVASGFTFSNYWMNWVRQSPEKGLEWVAEIRLKSNNYATHYAESVKGRFTISRDDSKSSVYLQMNNLRAEDTGIYYCTGVGQFAYWGQGTTVTVSSGGGGSGGGGSGGGGSDIELTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKL

SEQ ID NO: 33 - protein, 243 aa, Artificial Sequence
  Synthetic Construct
EIVLTQSPATLSLSPGERATLTCSATSSVSYIHWYQQRPGQSPRLLIYSTSNLASGIPARFSGSGSGSDYTLTISSLEPEDFAVYYCQQRSSSPFTFGSGTKVEIKGGGGSGGGGSGGGGSEVQLVESGGGLVKPGGSLRLSCAASGFTFSRYGMSWVRQAPGKRLEWVSTISGGGTYIYYPDSVKGRFTISRDNAKNTLYLQMNSLRAEDTAVYYCTRDNYGRNYDYGMDYWGQGTLVTVSS

(19) United States
(12) Patent Application Publication
(10) Pub. No.: US 2023/0203168 A1
(43) Pub. Date: Jun. 29, 2023

(54) Dual EGFR-MUC1 Chimeric Antigen Receptor T Cells

(71) Applicant: H. LEE MOFFITT CANCER CENTER AND RESEARCH INSTITUTE, INC., Tampa, FL (US)

(72) Inventor: Marco L. Davila, Tampa, FL (US)

(21) Appl. No.: 18/000,312

(22) PCT Filed: Jun. 1, 2021
(86) PCT No.: PCT/US21/35184

§ 371 Date: Nov. 30, 2022

Related U.S. Application Data
(60) Provisional application No. 63/033,392, filed on Jun. 2, 2020.

(51) Int. Cl. C07K 16/28; C07K 16/30; A61P 35/00

(52) U.S. Cl. CPC C07K 16/2863; C07K 16/3092; A61P 35/00; C07K 2319/02; C07K 2317/622; C07K 2317/56; C07K 2317/53; C07K 2317/24

(57) ABSTRACT

Bi-specific CAR-T cells are disclosed for treating NSCLCs. The disclosed CAR-T cells contain CAR polypeptides that can bind EGFR/MUC1-expressing cells. Therefore, also disclosed is an immune effector cell genetically modified to express an anti-EGFR CAR binding agent and an anti-MUC1 binding agent. Also disclosed are methods of providing an anti-tumor immunity in a subject with a EGFR and MUC1-expressing cancer that involves adoptive transfer of the disclosed immune effector cells engineered to express the disclosed CARs.

### CROSS-REFERENCE TO RELATED APPLICATIONS

This application claims benefit of U.S. Provisional Application No. 63/033,392, filed Jun. 2, 2020, which is hereby incorporated herein by reference in its entirety.

### SEQUENCE LISTING

This application contains a sequence listing filed in electronic form as an ASCII.txt file entitled “320803-2540 Sequence Listing_ST25” created on May 29, 2021 and having 23,190 bytes. The content of the sequence listing is incorporated herein in its entirety.

### BACKGROUND

Treatment with chimeric antigen receptor (CAR) engineered T cells is a novel therapeutic strategy that has shown great promise in treatment of selected malignancies. Pre-clinically, anti-tumor activity of CAR T cells has been noted in a variety of solid malignancies, but its role in clinical setting remains limited. This is due in part to a lack of appropriate tumor associated antigens and risk of on/off target toxicities associated with therapy. Recent advances in treatment of NSCLC including immunotherapy and targeted therapy have changed the landscape of treatment for patients with NSCLC. However, both treatment strategies have certain limitations.

### SUMMARY

Bi-specific CAR-T cells are disclosed for treating NSCLCs. The disclosed CAR-T cells contain CAR polypeptides that can bind EGFR/MUC1-expressing cells. Therefore, also disclosed is an immune effector cell genetically modified to express an anti-EGFR CAR binding agent and an anti-MUC1 binding agent.

The anti-EGFR or anti-MUC1 binding agent is in some embodiments an antibody fragment that specifically binds EGFR or MUC1. For example, the antigen binding domain can be a Fab or a single-chain variable fragment (scFv) of an antibody that specifically binds EGFR or MUC1. The anti-EGFR or anti-MUC1 binding agent is in some embodiments an aptamer that specifically binds EGFR or MUC1. For example, the anti-EGFR or anti-MUC1 binding agent can be a peptide aptamer selected from a random sequence pool based on its ability to bind EGFR or MUC1. The anti-EGFR or anti-MUC1 binding agent can also be a natural ligand of EGFR or MUC1, or a variant and/or fragment thereof capable of binding EGFR or MUC1.

In some embodiments, the anti-EGFR or anti-MUC1 scFv can comprise a variable heavy (V_{H}) domain having CDR1, CDR2 and CDR3 sequences and a variable light (V_{L}) domain having CDR1, CDR2 and CDR3 sequences.

Also disclosed herein is a bi-specific CAR polypeptide that includes a EGFR antigen binding domain, a MUC1 antigen binding domain, a transmembrane domain, an intracellular signaling domain, and a co-stimulatory signaling region. In some embodiments, the EGFR antigen binding domain is a single-chain variable fragment (scFv) of an antibody comprising a variable heavy (V_{H}) domain and a variable light (V_{L}) domain, and wherein the MUC1 antigen binding domain is a scFv comprising a V_{H} domain and a V_{L} domain.

As shown in FIG. 8, the bi-specific CAR polypeptide can have a tandem format and therefore be defined by the formula:

SP-EV_{H}-EV_{L}-MV_{H}-MV_{L}-HG-TM-CSR/IDS;

SP-EV_{L}-EV_{H}-MV_{H}-MV_{L}-HG-TM-CSR/IDS;

SP-EV_{H}-EV_{L}-MV_{L}-MV_{H}-HG-TM-CSR/IDS;

SP-EV_{L}-EV_{H}-MV_{L}-MV_{H}-HG-TM-CSR/IDS;

SP-MV_{H}-MV_{L}-EV_{H}-EV_{L}-HG-TM-CSR/IDS;

SP-MV_{L}-MV_{H}-EV_{H}-EV_{L}-HG-TM-CSR/IDS;

SP-MV_{H}-MV_{L}-EV_{L}-EV_{H}-HG-TM-CSR/IDS; or

SP-MV_{L}-MV_{H}-EV_{L}-EV_{H}-HG-TM-CSR/IDS;
wherein “SP” represents a signal peptide,
wherein “EV_{H}” represents the EGFR scFv V_{H} domain,
wherein “EV_{L}” represents the EGFR scFv V_{L} domain,
wherein “MV_{H}” represents the MUC1 scFv V_{H} domain,
wherein “MV_{L}” represents the MUC1 scFv V_{L} domain,
wherein “HG” represents and optional hinge domain,
wherein “TM” represents a transmembrane domain,
wherein “CSR/IDS” represents a co-stimulatory signaling region and an intracellular signaling domain,
wherein “-” represents a bivalent linker.

As shown in FIG. 8, the bi-specific CAR polypeptide can have a loop format and therefore be defined by the formula:

SP-EV_{H}-MV_{L}-MV_{H}-EV_{L}-HG-TM-CSR/IDS;

SP-EV_{L}-MV_{L}-MV_{H}-EV_{H}-HG-TM-CSR/IDS;

SP-EV_{H}-MV_{H}-MV_{L}-EV_{L}-HG-TM-CSR/IDS;

SP-EV_{L}-MV_{H}-MV_{L}-EV_{H}-HG-TM-CSR/IDS;

SP-MV_{H}-EV_{L}-EV_{H}-MV_{L}-HG-TM-CSR/IDS;

SP-MV_{L}-EV_{L}-EV_{H}-MV_{H}-HG-TM-CSR/IDS;

SP-MV_{H}-EV_{H}-EV_{L}-MV_{L}-HG-TM-CSR/IDS;

SP-MV_{L}-EV_{H}-EV_{L}-MV_{H}-HG-TM-CSR/IDS;
wherein “SP” represents a signal peptide,
wherein “EV_{H}” represents the EGFR scFv V_{H} domain,
wherein “EVA” represents the EGFR scFv V_{L} domain,
wherein “MV_{H}” represents the MUC1 scFv V_{H} domain,
wherein “MV_{L}” represents the MUC1 scFv V_{L} domain,
wherein “HG” represents and optional hinge domain,
wherein “TM” represents a transmembrane domain,
wherein “CSR/IDS” represents a co-stimulatory signaling region and an intracellular signaling domain,
wherein “-” represents a bivalent linker.

Anti-EGFR antibodies are disclosed in U.S. Pat. No. 8,580,263, which is incorporated by reference for the these antibodies, including sequences for use in preparing scFVs.

For example, in some embodiments of the anti-EGFR scFv, the CDR1 sequence of the V_{H} domain comprises the amino acid sequence KASGGTFSSYAIS (SEQ ID NO:1); CDR2 sequence of the V_{H} domain comprises the amino acid sequence GIIPIFGTANYAQKFQG (SEQ ID NO:2); CDR3 sequence of the V_{H} domain comprises the amino acid sequence AREEGPYCSSTSCYGAFDI (SEQ ID NO:3); CDR1 sequence of the V_{L} comprises the amino acid sequence QGDSLRSYFAS (SEQ ID NO:4); CDR2 sequence of the V_{L} domain comprises the amino acid sequence YARNDRPA (SEQ ID NO:5); and CDR3 sequence of the V_{L} domain comprises the amino acid sequence AAWDDSLNGYL (SEQ ID NO:6).

In some embodiments, the anti-EGFR scFv V_{H} domain comprises the amino acid sequence:

(SEQ ID NO: 7)
QVQLKQSGPGLVQPSQSLSITCTVSGFSLTNYGVHWRQSPGKGLEWLGV
IWSGGNTDYNTPFTSRLSINKDNSKSQVFFKMNSLQSNDTAIYYCARAL
TYYDYEFAYWGQGTLVTV.

In some embodiments, the anti-EGFR scFv V_{H} domain comprises the amino acid sequence:

(SEQ ID NO: 8)
EVQLVQSGAEVKKPGSSVKVSCKASGGTFSSYAISWRQAPGQGLEWMGG
IIPIFGTANYAQKFQGRVTITADESTSTAYMELSSLRSEDTAVYYCARE
EGPYCSSTSCYGAFDIWGQGTLVTVSS.

In some embodiments, the anti-EGFR scFv V_{L} domain comprises the amino acid sequence:

(SEQ ID NO: 9)
LLTQSPVILSVSPGERVSFSCRASQSIGTNIHWYQQRTNGSPRLLIKYA
SESISGIPSRFSGSGSGTDFTLSINSVESEDIADYYCQQNNNWPTTFGA
GTKLELKRTVA.

In some embodiments, the anti-EGFR scFv V_{L} domain comprises the amino acid sequence:

(SEQ ID NO: 10)
QSVLTQDPAVSVALGQTVKITCQGDSLRSYFASWYQQKPGQAPTLVMYG
VPDRFSGSKSGTSASLAISGLQSEDEADYYCAAWDDSLNGYLFGAGTKL
TVL.

In some embodiments, the anti-EGFR comprises an amino acid sequence:

(SEQ ID NO: 11)
EVQLVQSGAEVKKPGSSVKVSCKASGGTFSSYAISWRQAPGQGLEWMGG
IIPIFGTANYAQKFQGRVTITADESTSTAYMELSSLRSEDTAVYYCARE
EGPYCSSTSCYGAFDIWGQGTLVTVSSGGGGSGGGGSGGGGSQSVLTQD
PAVSVALGQTVKITCQGDSLRSYFASWYQQKPGQAPTLVMYGVPDRFSG
SKSGTSASLAISGLQSEDEADYYCAAWDDSLNGYLFGAGTKLTVL.

In some embodiments, the anti-EGFR comprises an amino acid sequence:

(SEQ ID NO: 12)
QSVLTQDPAVSVALGQTVKITCQGDSLRSYFASWYQQKPGQAPTLVMYG
VPDRFSGSKSGTSASLAISGLQSEDEADYYCAAWDDSLNGYLFGAGTKL
TVLGGGGSGGGGSGGGGSEVQLVQSGAEVKKPGSSVKVSCKASGGTFSS
YAISWRQAPGQGLEWMGGIIPIFGTANYAQKFQGRVTITADESTSTAYM
ELSSLRSEDTAVYYCAREEGPYCSSTSCYGAFDIWGQGTLVTVSS.

Anti-MUC1* antibodies are disclosed in U.S. Patent Publication 2017/0204191A1, which is incorporated by reference for these antibodies, including sequences for use in preparing scFVs.

In some embodiments of the anti-MUC1 scFv, the CDR1 sequence of the V_{H} domain comprises the amino acid sequence NYGMN (SEQ ID NO:13), GYAMS (SEQ ID NO:14), or R/GYA/GMS (SEQ ID NO:15); CDR2 sequence of the V_{H} domain comprises the amino acid sequence WINTYTGEPTYA/VG/DDFKG (SEQ ID NO:16) or TISSGGTYIYYPDSVKG (SEQ ID NO:17); CDR3 sequence of the V_{H} domain comprises the amino acid sequence S/TGT/DT/AXXY/FYA (SEQ ID NO:18), TGTTAILNG (SEQ ID NO:19), SGDGYWYYA (SEQ ID NO:20) or DNYGXXYDYG/A (SEQ ID NO:21); CDR1 sequence of the V_{L} comprises the amino acid sequence SASSSV/ISYM/IH/Y (SEQ ID NO:22) or RASKSVSTSGYSYMH (SEQ ID NO:23); CDR2 sequence of the V_{L} domain comprises the amino acid sequence S/GTSNLAS (SEQ ID NO:24) or LASNLES (SEQ ID NO:25); and CDR3 sequence of the V_{L} domain comprises the amino acid sequence QQRSS/NYPS/FT (SEQ ID NO:26) or QHSRELPFT (SEQ ID NO:27).

In some embodiments, the anti-MUC1 scFv V_{H} domain comprises the amino acid sequence:

(SEQ ID NO: 28)
VQLQESGGGLVQPGGSMKLSCVASGFTFSNYWMNWWRQSPEKGLEWAEI
RLKSNNYATHYAESVKGRFTISRDDSKSSVYLQMNNLRAEDTGIYYCTG
VGQFAYWGQGTTVTVSS.

In some embodiments, the anti-MUC1 scFv V_{H} domain comprises the amino acid sequence:

(SEQ ID NO: 29)
DIELTQESALTTSPGETVTLTCRSSTGAVTTSNYANWVQEKPDHLFTGL
IGGTNNRAPGVPARFSGSLIGDKAALTITGAQTEDEAIYFCALWYSNHW
WFGGGTKL.

In some embodiments, the anti-MUC1 scFv V_{L} domain comprises the amino acid sequence:

(SEQ ID NO: 30)
DIELTQESALTTSPGETVTLTCRSSTGAVTTSNYANWWQEKPDHLFTGL
IGGTNNRAPGVPARFSGSLIGDKAALTITGAQTEDEAIYFCALWYSNHW
WFGGGTKL.

In some embodiments, the anti-MUC1 scFv V_{L} domain comprises the amino acid sequence:

(SEQ ID NO: 31)
GGGGSVQLQESGGGLVQPGGSMKLSCVASGFTFSNYWMNWVRQSPEKGL
EVWAEIRLKSNNYATHYAESVKGRFTISRDDSKSSVYLQMNNLRAEDTG
IYYCTGVGQFAYWGQGTTVTVSS.

In some embodiments, the anti-MUC1 comprises an amino acid sequence:

(SEQ ID NO: 32)
VQLQESGGGLVQPGGSMKLSCVASGFTFSNYWMNWWRQSPEKGLEWAEI
RLKSNNYATHYAESVKGRFTISRDDSKSSVYLQMNNLRAEDTGIYYCTG
VGQFAYWGQGTTVTVSSGGGGSGGGGSGGGGSDIELTQESALTTSPGET
VTLTCRSSTGAVTTSNYANWWQEKPDHLFTGLIGGTNNRAPGVPARFSG
SLIGDKAALTITGAQTEDEAIYFCALWYSNHWVFGGGTKL.

In some embodiments, the anti-MUC1 comprises an amino acid sequence:

(SEQ ID NO: 33)
EIVLTQSPATLSLSPGERATLTCSATSSVSYIHWYQQRPGQSPRLLIYS
TSNLASGIPARFSGSGSGSDYTLTISSLEPEDFAVYYCQQRSSSPFTFG
SGTKVEIKGGGGSGGGGSGGGGSEVQLVESGGGLVKPGGSLRLSCAASG
FTFSRYGMSWWRQAPGKRLEWSTISGGGTYIYYPDSVKGRFTISRDNAK
NTLYLQMNSLRAEDTAVYYCTRDNYGRNYDYGMDYWGOGTLVTVSS.

As with other CARs, the disclosed polypeptides can also contain a transmembrane domain and an endodomain capable of activating an immune effector cell. For example, the endodomain can contain a signaling domain and one or more co-stimulatory signaling regions.

In some embodiments, the intracellular signaling domain is a CD3 zeta (CD3ζ) signaling domain. In some embodiments, the costimulatory signaling region comprises the cytoplasmic domain of CD28, 4-1BB, or a combination thereof. In some cases, the costimulatory signaling region contains 1, 2, 3, or 4 cytoplasmic domains of one or more intracellular signaling and/or costimulatory molecules. In some embodiments, the co-stimulatory signaling region contains one or more mutations in the cytoplasmic domains of CD28 and/or 4-1BB that enhance signaling.

In some embodiments, each of the CAR polypeptides in the bi-specific CAR-T cells contain an incomplete endodomain such that activation only occurs when both the anti-EGFR and anti-MUC1 CARs bind their respective antigens. For example, one of the CAR polypeptide can contain only an intracellular signaling domain and the other CAR polypeptide can contain only a co-stimulatory domain.

Therefore, in some embodiments, the anti-EGFR CAR polypeptide contains a CD3 zeta (CD3ζ) signaling domain but does not contain a costimulatory signaling region (CSR), and the anti-MUC1 CAR polypeptide contains the cytoplasmic domain of CD28, 4-1 BB, or a combination thereof, but does not contain a CD3 zeta (CD3) signaling domain (SD).

In other embodiments, the anti-EGFR CAR polypeptide contains the cytoplasmic domain of CD28, 4-1BB, or a combination thereof, but does not contain a CD3 zeta (CD3ζ) signaling domain (SD), and the MUC1 CAR polypeptide contains a CD3 zeta (CD3ζ) signaling domain but does not contain a costimulatory signaling region (CSR).

Also disclosed are isolated nucleic acid sequences encoding the disclosed CAR polypeptides, vectors comprising these isolated nucleic acids, and cells containing these vectors. For example, the cell can be an immune effector cell selected from the group consisting of an alpha-beta T cells, a gamma-delta T cell, a Natural Killer (NK) cells, a Natural Killer T (NKT) cell, a B cell, an innate lymphoid cell (ILC), a cytokine induced killer (CIK) cell, a cytotoxic T lymphocyte (CTL), a lymphokine activated killer (LAK) cell, and a regulatory T cell.

Also disclosed is a method of providing an anti-tumor immunity in a subject with a EGFR/MUC1-expressing cancer that involves administering to the subject an effective amount of an immune effector cell genetically modified with a disclosed EGFR/MUC1-specific CARs. In some cases, the cancer can be any EGFR/MUC1-expressing malignancy. In some cases, the cancer comprises NSCLC.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 shows NSCLC expresses both MUC1 and EGFR.

FIG. 2 shows EGFR and MUC1 CAR combinations.

FIGS. 3A to 3D show EGFR and MUC1 bi-specific CAR-T 1, 2, and 3 elicit effector response against NSCLC.

FIGS. 4A to 4C show different CARs do not show differences between different subsets of T cells.

FIG. 5 shows CAR T killing comparison on different cells. Activated Bi specific EGFR and MUC1 CAR T cells or mock transduced T cells were co-cultured with target NSCLC cell lines (H23, H460, H520, and PC9) and cytotoxicity was compared via xCELLigence system as mentioned before.

FIGS. 6A to 6D show all EGFR and MUC1 bi-specific CARs produce IFN-gamma cytokine against NSCLC cell lines. EGFR and MUC1 Bi-specific CART cell cytokine production. Activated Bi specific EGFR and MUC1 CAR T were co-cultured with indicated target cells for 24 hours. Supernatants were collected and cytokines were analyzed via Ella.

FIGS. 7A to 7D show EGFR and MUC1 Bi-specific CAR produces cytokine IL-6 against NSCLC cell lines. EGFR and MUC1 Bi-specific CART cell cytokine production. Activated Bi specific EGFR and MUC1 CAR T were co-cultured with indicated target cells for 24 hours. Supernatants were collected and IL-6 cytokine were analyzed via Ella.

FIG. 8 illustrates various bi-specific CAR polypeptide constructs.

### DETAILED DESCRIPTION

Bi-specific CAR-T cells are disclosed for treating NSCLCs. The disclosed CAR-T cells contain CAR polypeptides that can bind EGFR/MUC1-expressing cells. Therefore, also disclosed is an immune effector cell genetically modified to express an anti-EGFR CAR binding agent and an anti-MUC1 binding agent.

Chimeric Antigen Receptors (CAR)

CARs generally incorporate an antigen recognition domain from the single-chain variable fragments (scFv) of a monoclonal antibody (mAb) with transmembrane signaling motifs involved in lymphocyte activation (Sadelain M, et al. Nat Rev Cancer 2003 3:35-45). Disclosed herein are chimeric antigen receptor (CAR) that can be that can be expressed in immune effector cells to suppress alloreactive donor cells.

The disclosed CAR is generally made up of three domains: an ectodomain, a transmembrane domain, and an endodomain. The ectodomain comprises the EGFR or MUC1-binding region and is responsible for antigen recognition. It also optionally contains a signal peptide (SP) so that the CAR can be glycosylated and anchored in the cell membrane of the immune effector cell. The transmembrane domain (TD), is as its name suggests, connects the ectodomain to the endodomain and resides within the cell membrane when expressed by a cell. The endodomain is the business end of the CAR that transmits an activation signal to the immune effector cell after antigen recognition. For example, the endodomain can contain an intracellular signaling domain (ISD) and optionally a co-stimulatory signaling region (CSR).

A “signaling domain (SD)” generally contains immunoreceptor tyrosine-based activation motifs (ITAMs) that activate a signaling cascade when the ITAM is phosphorylated. The term “co-stimulatory signaling region (CSR)” refers to intracellular signaling domains from costimulatory protein receptors, such as CD28, 41BB, and ICOS, that are able to enhance T-cell activation by T-cell receptors.

In some embodiments, the endodomain contains an SD or a CSR, but not both. In these embodiments, an immune effector cell containing the disclosed CAR is only activated if another CAR (or a T-cell receptor) containing the missing domain also binds its respective antigen.

In some embodiments, the disclosed CAR is defined by the formula:

SP-ARD-HG-TM-CSR-SD; or

SP-ARD-HG-TM-SD-CSR;
wherein “SP” represents an optional signal peptide,
wherein “ARD” represents an antigen recognition domain,
wherein “HG” represents an optional hinge domain,
wherein “TM” represents a transmembrane domain,
wherein “CSR” represents one or more co-stimulatory signaling regions,
wherein “SD” represents a signaling domain, and
wherein “-” represents a peptide bond or linker.

Additional CAR constructs are described, for example, in Fresnak A D, et al. Engineered T cells: the promise and challenges of cancer immunotherapy. Nat Rev Cancer. 2016 Aug. 23; 16(9):566-81, which is incorporated by reference in its entirety for the teaching of these CAR models.

For example, the CAR can be a TRUCK, Universal CAR, Self-driving CAR, Armored CAR, Self-destruct CAR, Conditional CAR, Marked CAR, TanCAR, Dual CAR, or sCAR.

CAR T cells engineered to be resistant to immunosuppression (Armored CARs) may be genetically modified to no longer express various immune checkpoint molecules (for example, cytotoxic T lymphocyte-associated antigen 4 (CTLA4) or programmed cell death protein 1 (PD1)), with an immune checkpoint switch receptor, or may be administered with a monoclonal antibody that blocks immune checkpoint signaling.

A self-destruct CAR may be designed using RNA delivered by electroporation to encode the CAR. Alternatively, inducible apoptosis of the T cell may be achieved based on ganciclovir binding to thymidine kinase in gene-modified lymphocytes or the more recently described system of activation of human caspase 9 by a small-molecule dimerizer.

A conditional CAR T cell is by default unresponsive, or switched ‘off’, until the addition of a small molecule to complete the circuit, enabling full transduction of both signal 1 and signal 2, thereby activating the CAR T cell. Alternatively, T cells may be engineered to express an adaptor-specific receptor with affinity for subsequently administered secondary antibodies directed at target antigen.

A tandem CAR (TanCAR) T cell expresses a single CAR consisting of two linked single-chain variable fragments (scFvs) that have different affinities fused to intracellular co-stimulatory domain(s) and a CD3ζ domain. TanCAR T cell activation is achieved only when target cells co-express both targets.

A dual CAR T cell expresses two separate CARs with different ligand binding targets; one CAR includes only the CD3ζ domain and the other CAR includes only the co-stimulatory domain(s). Dual CAR T cell activation requires co-expression of both targets.

A safety CAR (sCAR) consists of an extracellular scFv fused to an intracellular inhibitory domain. sCAR T cells co-expressing a standard CAR become activated only when encountering target cells that possess the standard CAR target but lack the sCAR target.

The antigen recognition domain of the disclosed CAR is usually an scFv. There are however many alternatives. An antigen recognition domain from native T-cell receptor (TCR) alpha and beta single chains have been described, as have simple ectodomains (e.g. CD4 ectodomain to recognize HIV infected cells) and more exotic recognition components such as a linked cytokine (which leads to recognition of cells bearing the cytokine receptor). In fact almost anything that binds a given target with high affinity can be used as an antigen recognition region.

The endodomain is the business end of the CAR that after antigen recognition transmits a signal to the immune effector cell, activating at least one of the normal effector functions of the immune effector cell. Effector function of a T cell, for example, may be cytolytic activity or helper activity including the secretion of cytokines. Therefore, the endodomain may comprise the “intracellular signaling domain” of a T cell receptor (TCR) and optional co-receptors. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal.

Cytoplasmic signaling sequences that regulate primary activation of the TCR complex that act in a stimulatory manner may contain signaling motifs which are known as immunoreceptor tyrosine-based activation motifs (ITAMs). Examples of

ITAM containing cytoplasmic signaling sequences include those derived from CD8, CD3ζ, CD3δ, CD3γ, CD3ε, CD32 (Fc gamma RIIa), DAP10, DAP12, CD79a, CD79b, FcγRIγ, FcγRIIIγ, FcεRIβ (FCERIB), and FcεRIγ (FCERIG).

In particular embodiments, the intracellular signaling domain is derived from CD3 zeta (CD3ζ) (TCR zeta, GenBank accno. BAG36664.1). T-cell surface glycoprotein CD3 zeta (CD3ζ) chain, also known as T-cell receptor T3 zeta chain or CD247 (Cluster of Differentiation 247), is a protein that in humans is encoded by the CD247 gene.

First-generation CARs typically had the intracellular domain from the CD3ζ chain, which is the primary transmitter of signals from endogenous TCRs. Second-generation CARs add intracellular signaling domains from various costimulatory protein receptors (e.g., CD28, 41 BB, ICOS) to the endodomain of the CAR to provide additional signals to the T cell. More recent, third-generation CARs combine multiple signaling domains to further augment potency. T cells grafted with these CARs have demonstrated improved expansion, activation, persistence, and tumor-eradicating efficiency independent of costimulatory receptor/ligand interaction (Imai C, et al. Leukemia 2004 18:676-84; Maher J, et al. Nat Biotechnol 2002 20:70-5).

For example, the endodomain of the CAR can be designed to comprise the CD3ζ signaling domain by itself or combined with any other desired cytoplasmic domain(s) useful in the context of the CAR of the invention. For example, the cytoplasmic domain of the CAR can comprise a CD3ζ chain portion and a costimulatory signaling region. The costimulatory signaling region refers to a portion of the CAR comprising the intracellular domain of a costimulatory molecule. A costimulatory molecule is a cell surface molecule other than an antigen receptor or their ligands that is required for an efficient response of lymphocytes to an antigen. Examples of such molecules include CD27, CD28, 4-1BB (CD137), OX40, CD30, CD40, ICOS, lymphocyte function-associated antigen-1 (LFA-1), CD2, CD7, LIGHT, NKG2C, B7-H3, and a ligand that specifically binds with CD123, CD8, CD4, b2c, CD80, CD86, DAP10, DAP12, MyD88, BTNL3, and NKG2D. Thus, while the CAR is exemplified primarily with CD28 as the co-stimulatory signaling element, other costimulatory elements can be used alone or in combination with other co-stimulatory signaling elements.

In some embodiments, the CAR comprises a hinge sequence. A hinge sequence is a short sequence of amino acids that facilitates antibody flexibility (see, e.g., Woof et al., Nat. Rev. Immunol., 4(2): 89-99 (2004)). The hinge sequence may be positioned between the antigen recognition moiety (e.g., scFv) and the transmembrane domain. The hinge sequence can be any suitable sequence derived or obtained from any suitable molecule. In some embodiments, for example, the hinge sequence is derived from a CD8a molecule or a CD28 molecule.

The transmembrane domain may be derived either from a natural or from a synthetic source. Where the source is natural, the domain may be derived from any membrane-bound or transmembrane protein. For example, the transmembrane region may be derived from (i.e. comprise at least the transmembrane region(s) of) the alpha, beta or zeta chain of the T-cell receptor, CD28, CD3 epsilon, CD45, CD4, CD5, CD8 (e.g., CD8 alpha, CD8 beta), CD9, CD16, CD22, CD33, CD37, CD64, CD80, CD86, CD134, CD137, or CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2R beta, IL2R gamma, IL7R α, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, and PAG/Cbp. Alternatively the transmembrane domain may be synthetic, in which case it will comprise predominantly hydrophobic residues such as leucine and valine. In some cases, a triplet of phenylalanine, tryptophan and valine will be found at each end of a synthetic transmembrane domain. A short oligo- or polypeptide linker, such as between 2 and 10 amino acids in length, may form the linkage between the transmembrane domain and the endoplasmic domain of the CAR.

In some embodiments, the CAR has more than one transmembrane domain, which can be a repeat of the same transmembrane domain, or can be different transmembrane domains.

In some embodiments, the CAR is a multi-chain CAR, as described in WO2015/039523, which is incorporated by reference for this teaching. A multi-chain CAR can comprise separate extracellular ligand binding and signaling domains in different transmembrane polypeptides. The signaling domains can be designed to assemble in juxtamembrane position, which forms flexible architecture closer to natural receptors, that confers optimal signal transduction. For example, the multi-chain CAR can comprise a part of an FCERI alpha chain and a part of an FCERI beta chain such that the FCERI chains spontaneously dimerize together to form a CAR.

Tables 1, 2, and 3 below provide some example combinations of co-stimulatory signaling regions, and intracellular signaling domain that can occur in the disclosed CARs.

TABLE 1
First Generation CARs
Signal Domain

CD8
CD3ζ
CD3δ
CD3γ
CD3ε
FcγRI-γ
FcγRIII-γ
FcεRIβ
FcεRIγ
DAP10
DAP12
CD32
CD79a

TABLE 2
Second Generation CARs

| Co-stimulatory | Signal | Co-stimulatory | Signal |
|---|---|---|---|
| Signal | Domain | Signal | Domain |
| CD8 | CD8 | CD86 | FcεRIβ |
| CD8 | CD3ζ | CD86 | FcεRIγ |
| CD8 | CD3δ | CD86 | DAP10 |
| CD8 | CD3γ | CD86 | DAP12 |
| CD8 | CD3ε | CD86 | CD32 |
| CD8 | FcγRI-γ | CD86 | CD79a |
| CD8 | FcγRIII-γ | CD86 | CD79b |
| CD8 | FcεRIβ | OX40 | CD8 |
| CD8 | FcεRIγ | OX40 | CD3ζ |
| CD8 | DAP10 | OX40 | CD3δ |
| CD8 | DAP12 | OX40 | CD3γ |
| CD8 | CD32 | OX40 | CD3ε |
| CD8 | CD79a | OX40 | FcγRI-γ |
| CD8 | CD79b | OX40 | FcγRIII-γ |
| CD4 | CD8 | OX40 | FcεRIβ |
| CD4 | CD3ζ | OX40 | FcεRIγ |
| CD4 | CD3δ | OX40 | DAP10 |
| CD4 | CD3γ | OX40 | DAP12 |
| CD4 | CD3ε | OX40 | CD32 |
| CD4 | FcγRI-γ | OX40 | CD79a |
| CD4 | FcγRIII-γ | OX40 | CD79b |
| CD4 | FcεRIβ | DAP10 | CD8 |
| CD4 | FcεRIγ | DAP10 | CD3ζ |
| CD4 | DAP10 | DAP10 | CD3δ |
| CD4 | DAP12 | DAP10 | CD3γ |
| CD4 | CD32 | DAP10 | CD3ε |
| CD4 | CD79a | DAP10 | FcγRI-γ |
| CD4 | CD79b | DAP10 | FcγRIII-γ |
| b2c | CD8 | DAP10 | FcεRIβ |
| b2c | CD3ζ | DAP10 | FcεRIγ |
| b2c | CD3δ | DAP10 | DAP10 |
| b2c | CD3γ | DAP10 | DAP12 |
| b2c | CD3ε | DAP10 | CD32 |
| b2c | FcγRI-γ | DAP10 | CD79a |
| b2c | FcγRIII-γ | DAP10 | CD79b |
| b2c | FcεRIβ | DAP12 | CD8 |
| b2c | FcεRIγ | DAP12 | CD3ζ |
| b2c | DAP10 | DAP12 | CD3δ |
| b2c | DAP12 | DAP12 | CD3γ |
| b2c | CD32 | DAP12 | CD3ε |
| b2c | CD79a | DAP12 | FcγRI-γ |
| b2c | CD79b | DAP12 | FcγRIII-γ |
| CD137/41BB | CD8 | DAP12 | FcεRIβ |
| CD137/41BB | CD3ζ | DAP12 | FcεRIγ |
| CD137/41BB | CD3δ | DAP12 | DAP10 |
| CD137/41BB | CD3γ | DAP12 | DAP12 |
| CD137/41BB | CD3ε | DAP12 | CD32 |
| CD137/41BB | FcγRI-γ | DAP12 | CD79a |
| CD137/41BB | FcγRIII-γ | DAP12 | CD79b |
| CD137/41BB | FcεRIβ | MyD88 | CD8 |
| CD137/41BB | FcεRIγ | MyD88 | CD3ζ |
| CD137/41BB | DAP10 | MyD88 | CD3δ |
| CD137/41BB | DAP12 | MyD88 | CD3γ |
| CD137/41BB | CD32 | MyD88 | CD3ε |
| CD137/41BB | CD79a | MyD88 | FcγRI-γ |
| CD137/41BB | CD79b | MyD88 | FcγRIII-γ |
| ICOS | CD8 | MyD88 | FcεRIβ |
| ICOS | CD3ζ | MyD88 | FcεRIγ |
| ICOS | CD3δ | MyD88 | DAP10 |
| ICOS | CD3γ | MyD88 | DAP12 |
| ICOS | CD3ε | MyD88 | CD32 |
| ICOS | FcγRI-γ | MyD88 | CD79a |
| ICOS | FcγRIII-γ | MyD88 | CD79b |
| ICOS | FcεRIβ | CD7 | CD8 |
| ICOS | FcεRIγ | CD7 | CD3ζ |
| ICOS | DAP10 | CD7 | CD3δ |
| ICOS | DAP12 | CD7 | CD3γ |
| ICOS | CD32 | CD7 | CD3ε |
| ICOS | CD79a | CD7 | FcγRI-γ |
| ICOS | CD79b | CD7 | FcγRIII-γ |
| CD27 | CD8 | CD7 | FcεRIβ |
| CD27 | CD3ζ | CD7 | FcεRIγ |
| CD27 | CD3δ | CD7 | DAP10 |
| CD27 | CD3γ | CD7 | DAP12 |
| CD27 | CD3ε | CD7 | CD32 |
| CD27 | FcγRI-γ | CD7 | CD79a |
| CD27 | FcγRIII-γ | CD7 | CD79b |
| CD27 | FcεRIβ | BTNL3 | CD8 |
| CD27 | FcεRIγ | BTNL3 | CD3ζ |
| CD27 | DAP10 | BTNL3 | CD3δ |
| CD27 | DAP12 | BTNL3 | CD3γ |
| CD27 | CD32 | BTNL3 | CD3ε |
| CD27 | CD79a | BTNL3 | FcγRI-γ |
| CD27 | CD79b | BTNL3 | FcγRIII-γ |
| CD28δ | CD8 | BTNL3 | FcεRIβ |
| CD28δ | CD3ζ | BTNL3 | FcεRIγ |
| CD28δ | CD3δ | BTNL3 | DAP10 |
| CD28δ | CD3γ | BTNL3 | DAP12 |
| CD28δ | CD3ε | BTNL3 | CD32 |
| CD28δ | FcγRI-γ | BTNL3 | CD79a |
| CD28δ | FcγRIII-γ | BTNL3 | CD79b |
| CD28δ | FcεRIβ | NKG2D | CD8 |
| CD28δ | FcεRIγ | NKG2D | CD3ζ |
| CD28δ | DAP10 | NKG2D | CD3δ |
| CD28δ | DAP12 | NKG2D | CD3γ |
| CD28δ | CD32 | NKG2D | CD3ε |
| CD28δ | CD79a | NKG2D | FcγRI-γ |
| CD28δ | CD79b | NKG2D | FcγRIII-γ |
| CD80 | CD8 | NKG2D | FcεRIβ |
| CD80 | CD3ζ | NKG2D | FcεRIγ |
| CD80 | CD3δ | NKG2D | DAP10 |
| CD80 | CD3γ | NKG2D | DAP12 |
| CD80 | CD3ε | NKG2D | CD32 |
| CD80 | FcγRI-γ | NKG2D | CD79a |
| CD80 | FcγRIII-γ | NKG2D | CD79b |

TABLE 3
Third Generation CARs

| Co-stimulatory | Co-stimulatory | Signal |
|---|---|---|
| Signal | Signal | Domain |
| CD28 | CD28 | CD8 |
| CD28 | CD28 | CD3ζ |
| CD28 | CD28 | CD3δ |
| CD28 | CD28 | CD3γ |
| CD28 | CD28 | CD3ε |
| CD28 | CD28 | FcγRI-γ |
| CD28 | CD28 | FcγRIII-γ |
| CD28 | CD28 | FcεRIβ |
| CD28 | CD28 | FcεRIγ |
| CD28 | CD28 | DAP10 |
| CD28 | CD28 | DAP12 |
| CD28 | CD28 | CD32 |
| CD28 | CD28 | CD79a |
| CD28 | CD28 | CD79b |
| CD28 | CD8 | CD8 |
| CD28 | CD8 | CD3ζ |
| CD28 | CD8 | CD3δ |
| CD28 | CD8 | CD3γ |
| CD28 | CD8 | CD3ε |
| CD28 | CD8 | FcγRI-γ |
| CD28 | CD8 | FcγRIII-γ |
| CD28 | CD8 | FcεRIβ |
| CD28 | CD8 | FcεRIγ |
| CD28 | CD8 | DAP10 |
| CD28 | CD8 | DAP12 |
| CD28 | CD8 | CD32 |
| CD28 | CD8 | CD79a |
| CD28 | CD8 | CD79b |
| CD28 | CD4 | CD8 |
| CD28 | CD4 | CD3ζ |
| CD28 | CD4 | CD3δ |
| CD28 | CD4 | CD3γ |
| CD28 | CD4 | CD3ε |
| CD28 | CD4 | FcγRI-γ |
| CD28 | CD4 | FcγRIII-γ |
| CD28 | CD4 | FcεRIβ |
| CD28 | CD4 | FcεRIγ |
| CD28 | CD4 | DAP10 |
| CD28 | CD4 | DAP12 |
| CD28 | CD4 | CD32 |
| CD28 | CD4 | CD79a |
| CD28 | CD4 | CD79b |
| CD28 | b2c | CD8 |
| CD28 | b2c | CD3ζ |
| CD28 | b2c | CD3δ |
| CD28 | b2c | CD3γ |
| CD28 | b2c | CD3ε |
| CD28 | b2c | FcγRI-γ |
| CD28 | b2c | FcγRIII-γ |
| CD28 | b2c | FcεRIβ |
| CD28 | b2c | FcεRIγ |
| CD28 | b2c | DAP10 |
| CD28 | b2c | DAP12 |
| CD28 | b2c | CD32 |
| CD28 | b2c | CD79a |
| CD28 | b2c | CD79b |
| CD28 | CD137/41BB | CD8 |
| CD28 | CD137/41BB | CD3ζ |
| CD28 | CD137/41BB | CD3δ |
| CD28 | CD137/41BB | CD3γ |
| CD28 | CD137/41BB | CD3ε |
| CD28 | CD137/41BB | FcγRI-γ |
| CD28 | CD137/41BB | FcγRIII-γ |
| CD28 | CD137/41BB | FcεRIβ |
| CD28 | CD137/41BB | FcεRIγ |
| CD28 | CD137/41BB | DAP10 |
| CD28 | CD137/41BB | DAP12 |
| CD28 | CD137/41BB | CD32 |
| CD28 | CD137/41BB | CD79a |
| CD28 | CD137/41BB | CD79b |
| CD28 | ICOS | CD8 |
| CD28 | ICOS | CD3ζ |
| CD28 | ICOS | CD3δ |
| CD28 | ICOS | CD3γ |
| CD28 | ICOS | CD3ε |
| CD28 | ICOS | FcγRI-γ |
| CD28 | ICOS | FcγRIII-γ |
| CD28 | ICOS | FcεRIβ |
| CD28 | ICOS | FcεRIγ |
| CD28 | ICOS | DAP10 |
| CD28 | ICOS | DAP12 |
| CD28 | ICOS | CD32 |
| CD28 | ICOS | CD79a |
| CD28 | ICOS | CD79b |
| CD28 | CD27 | CD8 |
| CD28 | CD27 | CD3ζ |
| CD28 | CD27 | CD3δ |
| CD28 | CD27 | CD3γ |
| CD28 | CD27 | CD3ε |
| CD28 | CD27 | FcγRI-γ |
| CD28 | CD27 | FcγRIII-γ |
| CD28 | CD27 | FcεRIβ |
| CD28 | CD27 | FcεRIγ |
| CD28 | CD27 | DAP10 |
| CD28 | CD27 | DAP12 |
| CD28 | CD27 | CD32 |
| CD28 | CD27 | CD79a |
| CD28 | CD27 | CD79b |
| CD28 | CD28δ | CD8 |
| CD28 | CD28δ | CD3ζ |
| CD28 | CD28δ | CD3δ |
| CD28 | CD28δ | CD3γ |
| CD28 | CD28δ | CD3ε |
| CD28 | CD28δ | FcγRI-γ |
| CD28 | CD28δ | FcγRIII-γ |
| CD28 | CD28δ | FcεRIβ |
| CD28 | CD28δ | FcεRIγ |
| CD28 | CD28δ | DAP10 |
| CD28 | CD28δ | DAP12 |
| CD28 | CD28δ | CD32 |
| CD28 | CD28δ | CD79a |
| CD28 | CD28δ | CD79b |
| CD28 | CD80 | CD8 |
| CD28 | CD80 | CD3ζ |
| CD28 | CD80 | CD3δ |
| CD28 | CD80 | CD3γ |
| CD28 | CD80 | CD3ε |
| CD28 | CD80 | FcγRI-γ |
| CD28 | CD80 | FcγRIII-γ |
| CD28 | CD80 | FcεRIβ |
| CD28 | CD80 | FcεRIγ |
| CD28 | CD80 | DAP10 |
| CD28 | CD80 | DAP12 |
| CD28 | CD80 | CD32 |
| CD28 | CD80 | CD79a |
| CD28 | CD80 | CD79b |
| CD28 | CD86 | CD8 |
| CD28 | CD86 | CD3ζ |
| CD28 | CD86 | CD3δ |
| CD28 | CD86 | CD3γ |
| CD28 | CD86 | CD3ε |
| CD28 | CD86 | FcγRI-γ |
| CD28 | CD86 | FcγRIII-γ |
| CD28 | CD86 | FcεRIβ |
| CD28 | CD86 | FcεRIγ |
| CD28 | CD86 | DAP10 |
| CD28 | CD86 | DAP12 |
| CD28 | CD86 | CD32 |
| CD28 | CD86 | CD79a |
| CD28 | CD86 | CD79b |
| CD28 | OX40 | CD8 |
| CD28 | OX40 | CD3ζ |
| CD28 | OX40 | CD3δ |
| CD28 | OX40 | CD3γ |
| CD28 | OX40 | CD3ε |
| CD28 | OX40 | FcγRI-γ |
| CD28 | OX40 | FcγRIII-γ |
| CD28 | OX40 | FcεRIβ |
| CD28 | OX40 | FcεRIγ |
| CD28 | OX40 | DAP10 |
| CD28 | OX40 | DAP12 |
| CD28 | OX40 | CD32 |
| CD28 | OX40 | CD79a |
| CD28 | OX40 | CD79b |
| CD28 | DAP10 | CD8 |
| CD28 | DAP10 | CD3ζ |
| CD28 | DAP10 | CD3δ |
| CD28 | DAP10 | CD3γ |
| CD28 | DAP10 | CD3ε |
| CD28 | DAP10 | FcγRI-γ |
| CD28 | DAP10 | FcγRIII-γ |
| CD28 | DAP10 | FcεRIβ |
| CD28 | DAP10 | FcεRIγ |
| CD28 | DAP10 | DAP10 |
| CD28 | DAP10 | DAP12 |
| CD28 | DAP10 | CD32 |
| CD28 | DAP10 | CD79a |
| CD28 | DAP10 | CD79b |
| CD28 | DAP12 | CD8 |
| CD28 | DAP12 | CD3ζ |
| CD28 | DAP12 | CD3δ |
| CD28 | DAP12 | CD3γ |
| CD28 | DAP12 | CD3ε |
| CD28 | DAP12 | FcγRI-γ |
| CD28 | DAP12 | FcγRIII-γ |
| CD28 | DAP12 | FcεRIβ |
| CD28 | DAP12 | FcεRIγ |
| CD28 | DAP12 | DAP10 |
| CD28 | DAP12 | DAP12 |
| CD28 | DAP12 | CD32 |
| CD28 | DAP12 | CD79a |
| CD28 | DAP12 | CD79b |
| CD28 | MyD88 | CD8 |
| CD28 | MyD88 | CD3ζ |
| CD28 | MyD88 | CD3δ |
| CD28 | MyD88 | CD3γ |
| CD28 | MyD88 | CD3ε |
| CD28 | MyD88 | FcγRI-γ |
| CD28 | MyD88 | FcγRIII-γ |
| CD28 | MyD88 | FcεRIβ |
| CD28 | MyD88 | FcεRIγ |
| CD28 | MyD88 | DAP10 |
| CD28 | MyD88 | DAP12 |
| CD28 | MyD88 | CD32 |
| CD28 | MyD88 | CD79a |
| CD28 | MyD88 | CD79b |
| CD28 | CD7 | CD8 |
| CD28 | CD7 | CD3ζ |
| CD28 | CD7 | CD3δ |
| CD28 | CD7 | CD3γ |
| CD28 | CD7 | CD3ε |
| CD28 | CD7 | FcγRI-γ |
| CD28 | CD7 | FcγRIII-γ |
| CD28 | CD7 | FcεRIβ |
| CD28 | CD7 | FcεRIγ |
| CD28 | CD7 | DAP10 |
| CD28 | CD7 | DAP12 |
| CD28 | CD7 | CD32 |
| CD28 | CD7 | CD79a |
| CD28 | CD7 | CD79b |
| CD28 | BTNL3 | CD8 |
| CD28 | BTNL3 | CD3ζ |
| CD28 | BTNL3 | CD3δ |
| CD28 | BTNL3 | CD3γ |
| CD28 | BTNL3 | CD3ε |
| CD28 | BTNL3 | FcγRI-γ |
| CD28 | BTNL3 | FcγRIII-γ |
| CD28 | BTNL3 | FcεRIβ |
| CD28 | BTNL3 | FcεRIγ |
| CD28 | BTNL3 | DAP10 |
| CD28 | BTNL3 | DAP12 |
| CD28 | BTNL3 | CD32 |
| CD28 | BTNL3 | CD79a |
| CD28 | BTNL3 | CD79b |
| CD28 | NKG2D | CD8 |
| CD28 | NKG2D | CD3ζ |
| CD28 | NKG2D | CD3δ |
| CD28 | NKG2D | CD3γ |
| CD28 | NKG2D | CD3ε |
| CD28 | NKG2D | FcγRI-γ |
| CD28 | NKG2D | FcγRIII-γ |
| CD28 | NKG2D | FcεRIβ |
| CD28 | NKG2D | FcεRIγ |
| CD28 | NKG2D | DAP10 |
| CD28 | NKG2D | DAP12 |
| CD28 | NKG2D | CD32 |
| CD28 | NKG2D | CD79a |
| CD28 | NKG2D | CD79b |
| CD8 | CD28 | CD8 |
| CD8 | CD28 | CD3ζ |
| CD8 | CD28 | CD3δ |
| CD8 | CD28 | CD3γ |
| CD8 | CD28 | CD3ε |
| CD8 | CD28 | FcγRI-γ |
| CD8 | CD28 | FcγRIII-γ |
| CD8 | CD28 | FcεRIβ |
| CD8 | CD28 | FcεRIγ |
| CD8 | CD28 | DAP10 |
| CD8 | CD28 | DAP12 |
| CD8 | CD28 | CD32 |
| CD8 | CD28 | CD79a |
| CD8 | CD28 | CD79b |
| CD8 | CD8 | CD8 |
| CD8 | CD8 | CD3ζ |
| CD8 | CD8 | CD3δ |
| CD8 | CD8 | CD3γ |
| CD8 | CD8 | CD3ε |
| CD8 | CD8 | FcγRI-γ |
| CD8 | CD8 | FcγRIII-γ |
| CD8 | CD8 | FcεRIβ |
| CD8 | CD8 | FcεRIγ |
| CD8 | CD8 | DAP10 |
| CD8 | CD8 | DAP12 |
| CD8 | CD8 | CD32 |
| CD8 | CD8 | CD79a |
| CD8 | CD8 | CD79b |
| CD8 | CD4 | CD8 |
| CD8 | CD4 | CD3ζ |
| CD8 | CD4 | CD3δ |
| CD8 | CD4 | CD3γ |
| CD8 | CD4 | CD3ε |
| CD8 | CD4 | FcγRI-γ |
| CD8 | CD4 | FcγRIII-γ |
| CD8 | CD4 | FcεRIβ |
| CD8 | CD4 | FcεRIγ |
| CD8 | CD4 | DAP10 |
| CD8 | CD4 | DAP12 |
| CD8 | CD4 | CD32 |
| CD8 | CD4 | CD79a |
| CD8 | CD4 | CD79b |
| CD8 | b2c | CD8 |
| CD8 | b2c | CD3ζ |
| CD8 | b2c | CD3δ |
| CD8 | b2c | CD3γ |
| CD8 | b2c | CD3ε |
| CD8 | b2c | FcγRI-γ |
| CD8 | b2c | FcγRIII-γ |
| CD8 | b2c | FcεRIβ |
| CD8 | b2c | FcεRIγ |
| CD8 | b2c | DAP10 |
| CD8 | b2c | DAP12 |
| CD8 | b2c | CD32 |
| CD8 | b2c | CD79a |
| CD8 | b2c | CD79b |
| CD8 | CD137/41BB | CD8 |
| CD8 | CD137/41BB | CD3ζ |
| CD8 | CD137/41BB | CD3δ |
| CD8 | CD137/41BB | CD3γ |
| CD8 | CD137/41BB | CD3ε |
| CD8 | CD137/41BB | FcγRI-γ |
| CD8 | CD137/41BB | FcγRIII-γ |
| CD8 | CD137/41BB | FcεRIβ |
| CD8 | CD137/41BB | FcεRIγ |
| CD8 | CD137/41BB | DAP10 |
| CD8 | CD137/41BB | DAP12 |
| CD8 | CD137/41BB | CD32 |
| CD8 | CD137/41BB | CD79a |
| CD8 | CD137/41BB | CD79b |
| CD8 | ICOS | CD8 |
| CD8 | ICOS | CD3ζ |
| CD8 | ICOS | CD3δ |
| CD8 | ICOS | CD3γ |
| CD8 | ICOS | CD3ε |
| CD8 | ICOS | FcγRI-γ |
| CD8 | ICOS | FcγRIII-γ |
| CD8 | ICOS | FcεRIβ |
| CD8 | ICOS | FcεRIγ |
| CD8 | ICOS | DAP10 |
| CD8 | ICOS | DAP12 |
| CD8 | ICOS | CD32 |
| CD8 | ICOS | CD79a |
| CD8 | ICOS | CD79b |
| CD8 | CD27 | CD8 |
| CD8 | CD27 | CD3ζ |
| CD8 | CD27 | CD3δ |
| CD8 | CD27 | CD3γ |
| CD8 | CD27 | CD3ε |
| CD8 | CD27 | FcγRI-γ |
| CD8 | CD27 | FcγRIII-γ |
| CD8 | CD27 | FcεRIβ |
| CD8 | CD27 | FcεRIγ |
| CD8 | CD27 | DAP10 |
| CD8 | CD27 | DAP12 |
| CD8 | CD27 | CD32 |
| CD8 | CD27 | CD79a |
| CD8 | CD27 | CD79b |
| CD8 | CD28δ | CD8 |
| CD8 | CD28δ | CD3ζ |
| CD8 | CD28δ | CD3δ |
| CD8 | CD28δ | CD3γ |
| CD8 | CD28δ | CD3ε |
| CD8 | CD28δ | FcγRI-γ |
| CD8 | CD28δ | FcγRIII-γ |
| CD8 | CD28δ | FcεRIβ |
| CD8 | CD28δ | FcεRIγ |
| CD8 | CD28δ | DAP10 |
| CD8 | CD28δ | DAP12 |
| CD8 | CD28δ | CD32 |
| CD8 | CD28δ | CD79a |
| CD8 | CD28δ | CD79b |
| CD8 | CD80 | CD8 |
| CD8 | CD80 | CD3ζ |
| CD8 | CD80 | CD3δ |
| CD8 | CD80 | CD3γ |
| CD8 | CD80 | CD3ε |
| CD8 | CD80 | FcγRI-γ |
| CD8 | CD80 | FcγRIII-γ |
| CD8 | CD80 | FcεRIβ |
| CD8 | CD80 | FcεRIγ |
| CD8 | CD80 | DAP10 |
| CD8 | CD80 | DAP12 |
| CD8 | CD80 | CD32 |
| CD8 | CD80 | CD79a |
| CD8 | CD80 | CD79b |
| CD8 | CD86 | CD8 |
| CD8 | CD86 | CD3ζ |
| CD8 | CD86 | CD3δ |
| CD8 | CD86 | CD3γ |
| CD8 | CD86 | CD3ε |
| CD8 | CD86 | FcγRI-γ |
| CD8 | CD86 | FcγRIII-γ |
| CD8 | CD86 | FcεRIβ |
| CD8 | CD86 | FcεRIγ |
| CD8 | CD86 | DAP10 |
| CD8 | CD86 | DAP12 |
| CD8 | CD86 | CD32 |
| CD8 | CD86 | CD79a |
| CD8 | CD86 | CD79b |
| CD8 | OX40 | CD8 |
| CD8 | OX40 | CD3ζ |
| CD8 | OX40 | CD3δ |
| CD8 | OX40 | CD3γ |
| CD8 | OX40 | CD3ε |
| CD8 | OX40 | FcγRI-γ |
| CD8 | OX40 | FcγRIII-γ |
| CD8 | OX40 | FcεRIβ |
| CD8 | OX40 | FcεRIγ |
| CD8 | OX40 | DAP10 |
| CD8 | OX40 | DAP12 |
| CD8 | OX40 | CD32 |
| CD8 | OX40 | CD79a |
| CD8 | OX40 | CD79b |
| CD8 | DAP10 | CD8 |
| CD8 | DAP10 | CD3ζ |
| CD8 | DAP10 | CD3δ |
| CD8 | DAP10 | CD3γ |
| CD8 | DAP10 | CD3ε |
| CD8 | DAP10 | FcγRI-γ |
| CD8 | DAP10 | FcγRIII-γ |
| CD8 | DAP10 | FcεRIβ |
| CD8 | DAP10 | FcεRIγ |
| CD8 | DAP10 | DAP10 |
| CD8 | DAP10 | DAP12 |
| CD8 | DAP10 | CD32 |
| CD8 | DAP10 | CD79a |
| CD8 | DAP10 | CD79b |
| CD8 | DAP12 | CD8 |
| CD8 | DAP12 | CD3ζ |
| CD8 | DAP12 | CD3δ |
| CD8 | DAP12 | CD3γ |
| CD8 | DAP12 | CD3ε |
| CD8 | DAP12 | FcγRI-γ |
| CD8 | DAP12 | FcγRIII-γ |
| CD8 | DAP12 | FcεRIβ |
| CD8 | DAP12 | FcεRIγ |
| CD8 | DAP12 | DAP10 |
| CD8 | DAP12 | DAP12 |
| CD8 | DAP12 | CD32 |
| CD8 | DAP12 | CD79a |
| CD8 | DAP12 | CD79b |
| CD8 | MyD88 | CD8 |
| CD8 | MyD88 | CD3ζ |
| CD8 | MyD88 | CD3δ |
| CD8 | MyD88 | CD3γ |
| CD8 | MyD88 | CD3ε |
| CD8 | MyD88 | FcγRI-γ |
| CD8 | MyD88 | FcγRIII-γ |
| CD8 | MyD88 | FcεRIβ |
| CD8 | MyD88 | FcεRIγ |
| CD8 | MyD88 | DAP10 |
| CD8 | MyD88 | DAP12 |
| CD8 | MyD88 | CD32 |
| CD8 | MyD88 | CD79a |
| CD8 | MyD88 | CD79b |
| CD8 | CD7 | CD8 |
| CD8 | CD7 | CD3ζ |
| CD8 | CD7 | CD3δ |
| CD8 | CD7 | CD3γ |
| CD8 | CD7 | CD3ε |
| CD8 | CD7 | FcγRI-γ |
| CD8 | CD7 | FcγRIII-γ |
| CD8 | CD7 | FcεRIβ |
| CD8 | CD7 | FcεRIγ |
| CD8 | CD7 | DAP10 |
| CD8 | CD7 | DAP12 |
| CD8 | CD7 | CD32 |
| CD8 | CD7 | CD79a |
| CD8 | CD7 | CD79b |
| CD8 | BTNL3 | CD8 |
| CD8 | BTNL3 | CD3ζ |
| CD8 | BTNL3 | CD3δ |
| CD8 | BTNL3 | CD3γ |
| CD8 | BTNL3 | CD3ε |
| CD8 | BTNL3 | FcγRI-γ |
| CD8 | BTNL3 | FcγRIII-γ |
| CD8 | BTNL3 | FcεRIβ |
| CD8 | BTNL3 | FcεRIγ |
| CD8 | BTNL3 | DAP10 |
| CD8 | BTNL3 | DAP12 |
| CD8 | BTNL3 | CD32 |
| CD8 | BTNL3 | CD79a |
| CD8 | BTNL3 | CD79b |
| CD8 | NKG2D | CD8 |
| CD8 | NKG2D | CD3ζ |
| CD8 | NKG2D | CD3δ |
| CD8 | NKG2D | CD3γ |
| CD8 | NKG2D | CD3ε |
| CD8 | NKG2D | FcγRI-γ |
| CD8 | NKG2D | FcγRIII-γ |
| CD8 | NKG2D | FcεRIβ |
| CD8 | NKG2D | FcεRIγ |
| CD8 | NKG2D | DAP10 |
| CD8 | NKG2D | DAP12 |
| CD8 | NKG2D | CD32 |
| CD8 | NKG2D | CD79a |
| CD8 | NKG2D | CD79b |
| CD4 | CD28 | CD8 |
| CD4 | CD28 | CD3ζ |
| CD4 | CD28 | CD3δ |
| CD4 | CD28 | CD3γ |
| CD4 | CD28 | CD3ε |
| CD4 | CD28 | FcγRI-γ |
| CD4 | CD28 | FcγRIII-γ |
| CD4 | CD28 | FcεRIβ |
| CD4 | CD28 | FcεRIγ |
| CD4 | CD28 | DAP10 |
| CD4 | CD28 | DAP12 |
| CD4 | CD28 | CD32 |
| CD4 | CD28 | CD79a |
| CD4 | CD28 | CD79b |
| CD4 | CD8 | CD8 |
| CD4 | CD8 | CD3ζ |
| CD4 | CD8 | CD3δ |
| CD4 | CD8 | CD3γ |
| CD4 | CD8 | CD3ε |
| CD4 | CD8 | FcγRI-γ |
| CD4 | CD8 | FcγRIII-γ |
| CD4 | CD8 | FcεRIβ |
| CD4 | CD8 | FcεRIγ |
| CD4 | CD8 | DAP10 |
| CD4 | CD8 | DAP12 |
| CD4 | CD8 | CD32 |
| CD4 | CD8 | CD79a |
| CD4 | CD8 | CD79b |
| CD4 | CD4 | CD8 |
| CD4 | CD4 | CD3ζ |
| CD4 | CD4 | CD3δ |
| CD4 | CD4 | CD3γ |
| CD4 | CD4 | CD3ε |
| CD4 | CD4 | FcγRI-γ |
| CD4 | CD4 | FcγRIII-γ |
| CD4 | CD4 | FcεRIβ |
| CD4 | CD4 | FcεRIγ |
| CD4 | CD4 | DAP10 |
| CD4 | CD4 | DAP12 |
| CD4 | CD4 | CD32 |
| CD4 | CD4 | CD79a |
| CD4 | CD4 | CD79b |
| CD4 | b2c | CD8 |
| CD4 | b2c | CD3ζ |
| CD4 | b2c | CD3δ |
| CD4 | b2c | CD3γ |
| CD4 | b2c | CD3ε |
| CD4 | b2c | FcγRI-γ |
| CD4 | b2c | FcγRIII-γ |
| CD4 | b2c | FcεRIβ |
| CD4 | b2c | FcεRIγ |
| CD4 | b2c | DAP10 |
| CD4 | b2c | DAP12 |
| CD4 | b2c | CD32 |
| CD4 | b2c | CD79a |
| CD4 | b2c | CD79b |
| CD4 | CD137/41BB | CD8 |
| CD4 | CD137/41BB | CD3ζ |
| CD4 | CD137/41BB | CD3δ |
| CD4 | CD137/41BB | CD3γ |
| CD4 | CD137/41BB | CD3ε |
| CD4 | CD137/41BB | FcγRI-γ |
| CD4 | CD137/41BB | FcγRIII-γ |
| CD4 | CD137/41BB | FcεRIβ |
| CD4 | CD137/41BB | FcεRIγ |
| CD4 | CD137/41BB | DAP10 |
| CD4 | CD137/41BB | DAP12 |
| CD4 | CD137/41BB | CD32 |
| CD4 | CD137/41BB | CD79a |
| CD4 | CD137/41BB | CD79b |
| CD4 | ICOS | CD8 |
| CD4 | ICOS | CD3ζ |
| CD4 | ICOS | CD3δ |
| CD4 | ICOS | CD3γ |
| CD4 | ICOS | CD3ε |
| CD4 | ICOS | FcγRI-γ |
| CD4 | ICOS | FcγRIII-γ |
| CD4 | ICOS | FcεRIβ |
| CD4 | ICOS | FcεRIγ |
| CD4 | ICOS | DAP10 |
| CD4 | ICOS | DAP12 |
| CD4 | ICOS | CD32 |
| CD4 | ICOS | CD79a |
| CD4 | ICOS | CD79b |
| CD4 | CD27 | CD8 |
| CD4 | CD27 | CD3ζ |
| CD4 | CD27 | CD3δ |
| CD4 | CD27 | CD3γ |
| CD4 | CD27 | CD3ε |
| CD4 | CD27 | FcγRI-γ |
| CD4 | CD27 | FcγRIII-γ |
| CD4 | CD27 | FcεRIβ |
| CD4 | CD27 | FcεRIγ |
| CD4 | CD27 | DAP10 |
| CD4 | CD27 | DAP12 |
| CD4 | CD27 | CD32 |
| CD4 | CD27 | CD79a |
| CD4 | CD27 | CD79b |
| CD4 | CD28δ | CD8 |
| CD4 | CD28δ | CD3ζ |
| CD4 | CD28δ | CD3δ |
| CD4 | CD28δ | CD3γ |
| CD4 | CD28δ | CD3ε |
| CD4 | CD28δ | FcγRI-γ |
| CD4 | CD28δ | FcγRIII-γ |
| CD4 | CD28δ | FcεRIβ |
| CD4 | CD28δ | FcεRIγ |
| CD4 | CD28δ | DAP10 |
| CD4 | CD28δ | DAP12 |
| CD4 | CD28δ | CD32 |
| CD4 | CD28δ | CD79a |
| CD4 | CD28δ | CD79b |
| CD4 | CD80 | CD8 |
| CD4 | CD80 | CD3ζ |
| CD4 | CD80 | CD3δ |
| CD4 | CD80 | CD3γ |
| CD4 | CD80 | CD3ε |
| CD4 | CD80 | FcγRI-γ |
| CD4 | CD80 | FcγRIII-γ |
| CD4 | CD80 | FcεRIβ |
| CD4 | CD80 | FcεRIγ |
| CD4 | CD80 | DAP10 |
| CD4 | CD80 | DAP12 |
| CD4 | CD80 | CD32 |
| CD4 | CD80 | CD79a |
| CD4 | CD80 | CD79b |
| CD4 | CD86 | CD8 |
| CD4 | CD86 | CD3ζ |
| CD4 | CD86 | CD3δ |
| CD4 | CD86 | CD3γ |
| CD4 | CD86 | CD3ε |
| CD4 | CD86 | FcγRI-γ |
| CD4 | CD86 | FcγRIII-γ |
| CD4 | CD86 | FcεRIβ |
| CD4 | CD86 | FcεRIγ |
| CD4 | CD86 | DAP10 |
| CD4 | CD86 | DAP12 |
| CD4 | CD86 | CD32 |
| CD4 | CD86 | CD79a |
| CD4 | CD86 | CD79b |
| CD4 | OX40 | CD8 |
| CD4 | OX40 | CD3ζ |
| CD4 | OX40 | CD3δ |
| CD4 | OX40 | CD3γ |
| CD4 | OX40 | CD3ε |
| CD4 | OX40 | FcγRI-γ |
| CD4 | OX40 | FcγRIII-γ |
| CD4 | OX40 | FcεRIβ |
| CD4 | OX40 | FcεRIγ |
| CD4 | OX40 | DAP10 |
| CD4 | OX40 | DAP12 |
| CD4 | OX40 | CD32 |
| CD4 | OX40 | CD79a |
| CD4 | OX40 | CD79b |
| CD4 | DAP10 | CD8 |
| CD4 | DAP10 | CD3ζ |
| CD4 | DAP10 | CD3δ |
| CD4 | DAP10 | CD3γ |
| CD4 | DAP10 | CD3ε |
| CD4 | DAP10 | FcγRI-γ |
| CD4 | DAP10 | FcγRIII-γ |
| CD4 | DAP10 | FcεRIβ |
| CD4 | DAP10 | FcεRIγ |
| CD4 | DAP10 | DAP10 |
| CD4 | DAP10 | DAP12 |
| CD4 | DAP10 | CD32 |
| CD4 | DAP10 | CD79a |
| CD4 | DAP10 | CD79b |
| CD4 | DAP12 | CD8 |
| CD4 | DAP12 | CD3ζ |
| CD4 | DAP12 | CD3δ |
| CD4 | DAP12 | CD3γ |
| CD4 | DAP12 | CD3ε |
| CD4 | DAP12 | FcγRI-γ |
| CD4 | DAP12 | FcγRIII-γ |
| CD4 | DAP12 | FcεRIβ |
| CD4 | DAP12 | FcεRIγ |
| CD4 | DAP12 | DAP10 |
| CD4 | DAP12 | DAP12 |
| CD4 | DAP12 | CD32 |
| CD4 | DAP12 | CD79a |
| CD4 | DAP12 | CD79b |
| CD4 | MyD88 | CD8 |
| CD4 | MyD88 | CD3ζ |
| CD4 | MyD88 | CD3δ |
| CD4 | MyD88 | CD3γ |
| CD4 | MyD88 | CD3ε |
| CD4 | MyD88 | FcγRI-γ |
| CD4 | MyD88 | FcγRIII-γ |
| CD4 | MyD88 | FcεRIβ |
| CD4 | MyD88 | FcεRIγ |
| CD4 | MyD88 | DAP10 |
| CD4 | MyD88 | DAP12 |
| CD4 | MyD88 | CD32 |
| CD4 | MyD88 | CD79a |
| CD4 | MyD88 | CD79b |
| CD4 | CD7 | CD8 |
| CD4 | CD7 | CD3ζ |
| CD4 | CD7 | CD3δ |
| CD4 | CD7 | CD3γ |
| CD4 | CD7 | CD3ε |
| CD4 | CD7 | FcγRI-γ |
| CD4 | CD7 | FcγRIII-γ |
| CD4 | CD7 | FcεRIβ |
| CD4 | CD7 | FcεRIγ |
| CD4 | CD7 | DAP10 |
| CD4 | CD7 | DAP12 |
| CD4 | CD7 | CD32 |
| CD4 | CD7 | CD79a |
| CD4 | CD7 | CD79b |
| CD4 | BTNL3 | CD8 |
| CD4 | BTNL3 | CD3ζ |
| CD4 | BTNL3 | CD3δ |
| CD4 | BTNL3 | CD3γ |
| CD4 | BTNL3 | CD3ε |
| CD4 | BTNL3 | FcγRI-γ |
| CD4 | BTNL3 | FcγRIII-γ |
| CD4 | BTNL3 | FcεRIβ |
| CD4 | BTNL3 | FcεRIγ |
| CD4 | BTNL3 | DAP10 |
| CD4 | BTNL3 | DAP12 |
| CD4 | BTNL3 | CD32 |
| CD4 | BTNL3 | CD79a |
| CD4 | BTNL3 | CD79b |
| CD4 | NKG2D | CD8 |
| CD4 | NKG2D | CD3ζ |
| CD4 | NKG2D | CD3δ |
| CD4 | NKG2D | CD3γ |
| CD4 | NKG2D | CD3ε |
| CD4 | NKG2D | FcγRI-γ |
| CD4 | NKG2D | FcγRIII-γ |
| CD4 | NKG2D | FcεRIβ |
| CD4 | NKG2D | FcεRIγ |
| CD4 | NKG2D | DAP10 |
| CD4 | NKG2D | DAP12 |
| CD4 | NKG2D | CD32 |
| CD4 | NKG2D | CD79a |
| CD4 | NKG2D | CD79b |
| b2c | CD28 | CD8 |
| b2c | CD28 | CD3ζ |
| b2c | CD28 | CD3δ |
| b2c | CD28 | CD3γ |
| b2c | CD28 | CD3ε |
| b2c | CD28 | FcγRI-γ |
| b2c | CD28 | FcγRIII-γ |
| b2c | CD28 | FcεRIβ |
| b2c | CD28 | FcεRIγ |
| b2c | CD28 | DAP10 |
| b2c | CD28 | DAP12 |
| b2c | CD28 | CD32 |
| b2c | CD28 | CD79a |
| b2c | CD28 | CD79b |
| b2c | CD8 | CD8 |
| b2c | CD8 | CD3ζ |
| b2c | CD8 | CD3δ |
| b2c | CD8 | CD3γ |
| b2c | CD8 | CD3ε |
| b2c | CD8 | FcγRI-γ |
| b2c | CD8 | FcγRIII-γ |
| b2c | CD8 | FcεRIβ |
| b2c | CD8 | FcεRIγ |
| b2c | CD8 | DAP10 |
| b2c | CD8 | DAP12 |
| b2c | CD8 | CD32 |
| b2c | CD8 | CD79a |
| b2c | CD8 | CD79b |
| b2c | CD4 | CD8 |
| b2c | CD4 | CD3ζ |
| b2c | CD4 | CD3δ |
| b2c | CD4 | CD3γ |
| b2c | CD4 | CD3ε |
| b2c | CD4 | FcγRI-γ |
| b2c | CD4 | FcγRIII-γ |
| b2c | CD4 | FcεRIβ |
| b2c | CD4 | FcεRIγ |
| b2c | CD4 | DAP10 |
| b2c | CD4 | DAP12 |
| b2c | CD4 | CD32 |
| b2c | CD4 | CD79a |
| b2c | CD4 | CD79b |
| b2c | b2c | CD8 |
| b2c | b2c | CD3ζ |
| b2c | b2c | CD3δ |
| b2c | b2c | CD3γ |
| b2c | b2c | CD3ε |
| b2c | b2c | FcγRI-γ |
| b2c | b2c | FcγRIII-γ |
| b2c | b2c | FcεRIβ |
| b2c | b2c | FcεRIγ |
| b2c | b2c | DAP10 |
| b2c | b2c | DAP12 |
| b2c | b2c | CD32 |
| b2c | b2c | CD79a |
| b2c | b2c | CD79b |
| b2c | CD137/41BB | CD8 |
| b2c | CD137/41BB | CD3ζ |
| b2c | CD137/41BB | CD3δ |
| b2c | CD137/41BB | CD3γ |
| b2c | CD137/41BB | CD3ε |
| b2c | CD137/41BB | FcγRI-γ |
| b2c | CD137/41BB | FcγRIII-γ |
| b2c | CD137/41BB | FcεRIβ |
| b2c | CD137/41BB | FcεRIγ |
| b2c | CD137/41BB | DAP10 |
| b2c | CD137/41BB | DAP12 |
| b2c | CD137/41BB | CD32 |
| b2c | CD137/41BB | CD79a |
| b2c | CD137/41BB | CD79b |
| b2c | ICOS | CD8 |
| b2c | ICOS | CD3ζ |
| b2c | ICOS | CD3δ |
| b2c | ICOS | CD3γ |
| b2c | ICOS | CD3ε |
| b2c | ICOS | FcγRI-γ |
| b2c | ICOS | FcγRIII-γ |
| b2c | ICOS | FcεRIβ |
| b2c | ICOS | FcεRIγ |
| b2c | ICOS | DAP10 |
| b2c | ICOS | DAP12 |
| b2c | ICOS | CD32 |
| b2c | ICOS | CD79a |
| b2c | ICOS | CD79b |
| b2c | CD27 | CD8 |
| b2c | CD27 | CD3ζ |
| b2c | CD27 | CD3δ |
| b2c | CD27 | CD3γ |
| b2c | CD27 | CD3ε |
| b2c | CD27 | FcγRI-γ |
| b2c | CD27 | FcγRIII-γ |
| b2c | CD27 | FcεRIβ |
| b2c | CD27 | FcεRIγ |
| b2c | CD27 | DAP10 |
| b2c | CD27 | DAP12 |
| b2c | CD27 | CD32 |
| b2c | CD27 | CD79a |
| b2c | CD27 | CD79b |
| b2c | CD28δ | CD8 |
| b2c | CD28δ | CD3ζ |
| b2c | CD28δ | CD3δ |
| b2c | CD28δ | CD3γ |
| b2c | CD28δ | CD3ε |
| b2c | CD28δ | FcγRI-γ |
| b2c | CD28δ | FcγRIII-γ |
| b2c | CD28δ | FcεRIβ |
| b2c | CD28δ | FcεRIγ |
| b2c | CD28δ | DAP10 |
| b2c | CD28δ | DAP12 |
| b2c | CD28δ | CD32 |
| b2c | CD28δ | CD79a |
| b2c | CD28δ | CD79b |
| b2c | CD80 | CD8 |
| b2c | CD80 | CD3ζ |
| b2c | CD80 | CD3δ |
| b2c | CD80 | CD3γ |
| b2c | CD80 | CD3ε |
| b2c | CD80 | FcγRI-γ |
| b2c | CD80 | FcγRIII-γ |
| b2c | CD80 | FcεRIβ |
| b2c | CD80 | FcεRIγ |
| b2c | CD80 | DAP10 |
| b2c | CD80 | DAP12 |
| b2c | CD80 | CD32 |
| b2c | CD80 | CD79a |
| b2c | CD80 | CD79b |
| b2c | CD86 | CD8 |
| b2c | CD86 | CD3ζ |
| b2c | CD86 | CD3δ |
| b2c | CD86 | CD3γ |
| b2c | CD86 | CD3ε |
| b2c | CD86 | FcγRI-γ |
| b2c | CD86 | FcγRIII-γ |
| b2c | CD86 | FcεRIβ |
| b2c | CD86 | FcεRIγ |
| b2c | CD86 | DAP10 |
| b2c | CD86 | DAP12 |
| b2c | CD86 | CD32 |
| b2c | CD86 | CD79a |
| b2c | CD86 | CD79b |
| b2c | OX40 | CD8 |
| b2c | OX40 | CD3ζ |
| b2c | OX40 | CD3δ |
| b2c | OX40 | CD3γ |
| b2c | OX40 | CD3ε |
| b2c | OX40 | FcγRI-γ |
| b2c | OX40 | FcγRIII-γ |
| b2c | OX40 | FcεRIβ |
| b2c | OX40 | FcεRIγ |
| b2c | OX40 | DAP10 |
| b2c | OX40 | DAP12 |
| b2c | OX40 | CD32 |
| b2c | OX40 | CD79a |
| b2c | OX40 | CD79b |
| b2c | DAP10 | CD8 |
| b2c | DAP10 | CD3ζ |
| b2c | DAP10 | CD3δ |
| b2c | DAP10 | CD3γ |
| b2c | DAP10 | CD3ε |
| b2c | DAP10 | FcγRI-γ |
| b2c | DAP10 | FcγRIII-γ |
| b2c | DAP10 | FcεRIβ |
| b2c | DAP10 | FcεRIγ |
| b2c | DAP10 | DAP10 |
| b2c | DAP10 | DAP12 |
| b2c | DAP10 | CD32 |
| b2c | DAP10 | CD79a |
| b2c | DAP10 | CD79b |
| b2c | DAP12 | CD8 |
| b2c | DAP12 | CD3ζ |
| b2c | DAP12 | CD3δ |
| b2c | DAP12 | CD3γ |
| b2c | DAP12 | CD3ε |
| b2c | DAP12 | FcγRI-γ |
| b2c | DAP12 | FcγRIII-γ |
| b2c | DAP12 | FcεRIβ |
| b2c | DAP12 | FcεRIγ |
| b2c | DAP12 | DAP10 |
| b2c | DAP12 | DAP12 |
| b2c | DAP12 | CD32 |
| b2c | DAP12 | CD79a |
| b2c | DAP12 | CD79b |
| b2c | MyD88 | CD8 |
| b2c | MyD88 | CD3ζ |
| b2c | MyD88 | CD3δ |
| b2c | MyD88 | CD3γ |
| b2c | MyD88 | CD3ε |
| b2c | MyD88 | FcγRI-γ |
| b2c | MyD88 | FcγRIII-γ |
| b2c | MyD88 | FcεRIβ |
| b2c | MyD88 | FcεRIγ |
| b2c | MyD88 | DAP10 |
| b2c | MyD88 | DAP12 |
| b2c | MyD88 | CD32 |
| b2c | MyD88 | CD79a |
| b2c | MyD88 | CD79b |
| b2c | CD7 | CD8 |
| b2c | CD7 | CD3ζ |
| b2c | CD7 | CD3δ |
| b2c | CD7 | CD3γ |
| b2c | CD7 | CD3ε |
| b2c | CD7 | FcγRI-γ |
| b2c | CD7 | FcγRIII-γ |
| b2c | CD7 | FcεRIβ |
| b2c | CD7 | FcεRIγ |
| b2c | CD7 | DAP10 |
| b2c | CD7 | DAP12 |
| b2c | CD7 | CD32 |
| b2c | CD7 | CD79a |
| b2c | CD7 | CD79b |
| b2c | BTNL3 | CD8 |
| b2c | BTNL3 | CD3ζ |
| b2c | BTNL3 | CD3δ |
| b2c | BTNL3 | CD3γ |
| b2c | BTNL3 | CD3ε |
| b2c | BTNL3 | FcγRI-γ |
| b2c | BTNL3 | FcγRIII-γ |
| b2c | BTNL3 | FcεRIβ |
| b2c | BTNL3 | FcεRIγ |
| b2c | BTNL3 | DAP10 |
| b2c | BTNL3 | DAP12 |
| b2c | BTNL3 | CD32 |
| b2c | BTNL3 | CD79a |
| b2c | BTNL3 | CD79b |
| b2c | NKG2D | CD8 |
| b2c | NKG2D | CD3ζ |
| b2c | NKG2D | CD3δ |
| b2c | NKG2D | CD3γ |
| b2c | NKG2D | CD3ε |
| b2c | NKG2D | FcγRI-γ |
| b2c | NKG2D | FcγRIII-γ |
| b2c | NKG2D | FcεRIβ |
| b2c | NKG2D | FcεRIγ |
| b2c | NKG2D | DAP10 |
| b2c | NKG2D | DAP12 |
| b2c | NKG2D | CD32 |
| b2c | NKG2D | CD79a |
| b2c | NKG2D | CD79b |
| CD137/41BB | CD28 | CD8 |
| CD137/41BB | CD28 | CD3ζ |
| CD137/41BB | CD28 | CD3δ |
| CD137/41BB | CD28 | CD3γ |
| CD137/41BB | CD28 | CD3ε |
| CD137/41BB | CD28 | FcγRI-γ |
| CD137/41BB | CD28 | FcγRIII-γ |
| CD137/41BB | CD28 | FcεRIβ |
| CD137/41BB | CD28 | FcεRIγ |
| CD137/41BB | CD28 | DAP10 |
| CD137/41BB | CD28 | DAP12 |
| CD137/41BB | CD28 | CD32 |
| CD137/41BB | CD28 | CD79a |
| CD137/41BB | CD28 | CD79b |
| CD137/41BB | CD8 | CD8 |
| CD137/41BB | CD8 | CD3ζ |
| CD137/41BB | CD8 | CD3δ |
| CD137/41BB | CD8 | CD3γ |
| CD137/41BB | CD8 | CD3ε |
| CD137/41BB | CD8 | FcγRI-γ |
| CD137/41BB | CD8 | FcγRIII-γ |
| CD137/41BB | CD8 | FcεRIβ |
| CD137/41BB | CD8 | FcεRIγ |
| CD137/41BB | CD8 | DAP10 |
| CD137/41BB | CD8 | DAP12 |
| CD137/41BB | CD8 | CD32 |
| CD137/41BB | CD8 | CD79a |
| CD137/41BB | CD8 | CD79b |
| CD137/41BB | CD4 | CD8 |
| CD137/41BB | CD4 | CD3ζ |
| CD137/41BB | CD4 | CD3δ |
| CD137/41BB | CD4 | CD3γ |
| CD137/41BB | CD4 | CD3ε |
| CD137/41BB | CD4 | FcγRI-γ |
| CD137/41BB | CD4 | FcγRIII-γ |
| CD137/41BB | CD4 | FcεRIβ |
| CD137/41BB | CD4 | FcεRIγ |
| CD137/41BB | CD4 | DAP10 |
| CD137/41BB | CD4 | DAP12 |
| CD137/41BB | CD4 | CD32 |
| CD137/41BB | CD4 | CD79a |
| CD137/41BB | CD4 | CD79b |
| CD137/41BB | b2c | CD8 |
| CD137/41BB | b2c | CD3ζ |
| CD137/41BB | b2c | CD3δ |
| CD137/41BB | b2c | CD3γ |
| CD137/41BB | b2c | CD3ε |
| CD137/41BB | b2c | FcγRI-γ |
| CD137/41BB | b2c | FcγRIII-γ |
| CD137/41BB | b2c | FcεRIβ |
| CD137/41BB | b2c | FcεRIγ |
| CD137/41BB | b2c | DAP10 |
| CD137/41BB | b2c | DAP12 |
| CD137/41BB | b2c | CD32 |
| CD137/41BB | b2c | CD79a |
| CD137/41BB | b2c | CD79b |
| CD137/41BB | CD137/41BB | CD8 |
| CD137/41BB | CD137/41BB | CD3ζ |
| CD137/41BB | CD137/41BB | CD3δ |
| CD137/41BB | CD137/41BB | CD3γ |
| CD137/41BB | CD137/41BB | CD3ε |
| CD137/41BB | CD137/41BB | FcγRI-γ |
| CD137/41BB | CD137/41BB | FcγRIII-γ |
| CD137/41BB | CD137/41BB | FcεRIβ |
| CD137/41BB | CD137/41BB | FcεRIγ |
| CD137/41BB | CD137/41BB | DAP10 |
| CD137/41BB | CD137/41BB | DAP12 |
| CD137/41BB | CD137/41BB | CD32 |
| CD137/41BB | CD137/41BB | CD79a |
| CD137/41BB | CD137/41BB | CD79b |
| CD137/41BB | ICOS | CD8 |
| CD137/41BB | ICOS | CD3ζ |
| CD137/41BB | ICOS | CD3δ |
| CD137/41BB | ICOS | CD3γ |
| CD137/41BB | ICOS | CD3ε |
| CD137/41BB | ICOS | FcγRI-γ |
| CD137/41BB | ICOS | FcγRIII-γ |
| CD137/41BB | ICOS | FcεRIβ |
| CD137/41BB | ICOS | FcεRIγ |
| CD137/41BB | ICOS | DAP10 |
| CD137/41BB | ICOS | DAP12 |
| CD137/41BB | ICOS | CD32 |
| CD137/41BB | ICOS | CD79a |
| CD137/41BB | ICOS | CD79b |
| CD137/41BB | CD27 | CD8 |
| CD137/41BB | CD27 | CD3ζ |
| CD137/41BB | CD27 | CD3δ |
| CD137/41BB | CD27 | CD3γ |
| CD137/41BB | CD27 | CD3ε |
| CD137/41BB | CD27 | FcγRI-γ |
| CD137/41BB | CD27 | FcγRIII-γ |
| CD137/41BB | CD27 | FcεRIβ |
| CD137/41BB | CD27 | FcεRIγ |
| CD137/41BB | CD27 | DAP10 |
| CD137/41BB | CD27 | DAP12 |
| CD137/41BB | CD27 | CD32 |
| CD137/41BB | CD27 | CD79a |
| CD137/41BB | CD27 | CD79b |
| CD137/41BB | CD28δ | CD8 |
| CD137/41BB | CD28δ | CD3ζ |
| CD137/41BB | CD28δ | CD3δ |
| CD137/41BB | CD28δ | CD3γ |
| CD137/41BB | CD28δ | CD3ε |
| CD137/41BB | CD28δ | FcγRI-γ |
| CD137/41BB | CD28δ | FcγRIII-γ |
| CD137/41BB | CD28δ | FcεRIβ |
| CD137/41BB | CD28δ | FcεRIγ |
| CD137/41BB | CD28δ | DAP10 |
| CD137/41BB | CD28δ | DAP12 |
| CD137/41BB | CD28δ | CD32 |
| CD137/41BB | CD28δ | CD79a |
| CD137/41BB | CD28δ | CD79b |
| CD137/41BB | CD80 | CD8 |
| CD137/41BB | CD80 | CD3ζ |
| CD137/41BB | CD80 | CD3δ |
| CD137/41BB | CD80 | CD3γ |
| CD137/41BB | CD80 | CD3ε |
| CD137/41BB | CD80 | FcγRI-γ |
| CD137/41BB | CD80 | FcγRIII-γ |
| CD137/41BB | CD80 | FcεRIβ |
| CD137/41BB | CD80 | FcεRIγ |
| CD137/41BB | CD80 | DAP10 |
| CD137/41BB | CD80 | DAP12 |
| CD137/41BB | CD80 | CD32 |
| CD137/41BB | CD80 | CD79a |
| CD137/41BB | CD80 | CD79b |
| CD137/41BB | CD86 | CD8 |
| CD137/41BB | CD86 | CD3ζ |
| CD137/41BB | CD86 | CD3δ |
| CD137/41BB | CD86 | CD3γ |
| CD137/41BB | CD86 | CD3ε |
| CD137/41BB | CD86 | FcγRI-γ |
| CD137/41BB | CD86 | FcγRIII-γ |
| CD137/41BB | CD86 | FcεRIβ |
| CD137/41BB | CD86 | FcεRIγ |
| CD137/41BB | CD86 | DAP10 |
| CD137/41BB | CD86 | DAP12 |
| CD137/41BB | CD86 | CD32 |
| CD137/41BB | CD86 | CD79a |
| CD137/41BB | CD86 | CD79b |
| CD137/41BB | OX40 | CD8 |
| CD137/41BB | OX40 | CD3ζ |
| CD137/41BB | OX40 | CD3δ |
| CD137/41BB | OX40 | CD3γ |
| CD137/41BB | OX40 | CD3ε |
| CD137/41BB | OX40 | FcγRI-γ |
| CD137/41BB | OX40 | FcγRIII-γ |
| CD137/41BB | OX40 | FcεRIβ |
| CD137/41BB | OX40 | FcεRIγ |
| CD137/41BB | OX40 | DAP10 |
| CD137/41BB | OX40 | DAP12 |
| CD137/41BB | OX40 | CD32 |
| CD137/41BB | OX40 | CD79a |
| CD137/41BB | OX40 | CD79b |
| CD137/41BB | DAP10 | CD8 |
| CD137/41BB | DAP10 | CD3ζ |
| CD137/41BB | DAP10 | CD3δ |
| CD137/41BB | DAP10 | CD3γ |
| CD137/41BB | DAP10 | CD3ε |
| CD137/41BB | DAP10 | FcγRI-γ |
| CD137/41BB | DAP10 | FcγRIII-γ |
| CD137/41BB | DAP10 | FcεRIβ |
| CD137/41BB | DAP10 | FcεRIγ |
| CD137/41BB | DAP10 | DAP10 |
| CD137/41BB | DAP10 | DAP12 |
| CD137/41BB | DAP10 | CD32 |
| CD137/41BB | DAP10 | CD79a |
| CD137/41BB | DAP10 | CD79b |
| CD137/41BB | DAP12 | CD8 |
| CD137/41BB | DAP12 | CD3ζ |
| CD137/41BB | DAP12 | CD3δ |
| CD137/41BB | DAP12 | CD3γ |
| CD137/41BB | DAP12 | CD3ε |
| CD137/41BB | DAP12 | FcγRI-γ |
| CD137/41BB | DAP12 | FcγRIII-γ |
| CD137/41BB | DAP12 | FcεRIβ |
| CD137/41BB | DAP12 | FcεRIγ |
| CD137/41BB | DAP12 | DAP10 |
| CD137/41BB | DAP12 | DAP12 |
| CD137/41BB | DAP12 | CD32 |
| CD137/41BB | DAP12 | CD79a |
| CD137/41BB | DAP12 | CD79b |
| CD137/41BB | MyD88 | CD8 |
| CD137/41BB | MyD88 | CD3ζ |
| CD137/41BB | MyD88 | CD3δ |
| CD137/41BB | MyD88 | CD3γ |
| CD137/41BB | MyD88 | CD3ε |
| CD137/41BB | MyD88 | FcγRI-γ |
| CD137/41BB | MyD88 | FcγRIII-γ |
| CD137/41BB | MyD88 | FcεRIβ |
| CD137/41BB | MyD88 | FcεRIγ |
| CD137/41BB | MyD88 | DAP10 |
| CD137/41BB | MyD88 | DAP12 |
| CD137/41BB | MyD88 | CD32 |
| CD137/41BB | MyD88 | CD79a |
| CD137/41BB | MyD88 | CD79b |
| CD137/41BB | CD7 | CD8 |
| CD137/41BB | CD7 | CD3ζ |
| CD137/41BB | CD7 | CD3δ |
| CD137/41BB | CD7 | CD3γ |
| CD137/41BB | CD7 | CD3ε |
| CD137/41BB | CD7 | FcγRI-γ |
| CD137/41BB | CD7 | FcγRIII-γ |
| CD137/41BB | CD7 | FcεRIβ |
| CD137/41BB | CD7 | FcεRIγ |
| CD137/41BB | CD7 | DAP10 |
| CD137/41BB | CD7 | DAP12 |
| CD137/41BB | CD7 | CD32 |
| CD137/41BB | CD7 | CD79a |
| CD137/41BB | CD7 | CD79b |
| CD137/41BB | BTNL3 | CD8 |
| CD137/41BB | BTNL3 | CD3ζ |
| CD137/41BB | BTNL3 | CD3δ |
| CD137/41BB | BTNL3 | CD3γ |
| CD137/41BB | BTNL3 | CD3ε |
| CD137/41BB | BTNL3 | FcγRI-γ |
| CD137/41BB | BTNL3 | FcγRIII-γ |
| CD137/41BB | BTNL3 | FcεRIβ |
| CD137/41BB | BTNL3 | FcεRIγ |
| CD137/41BB | BTNL3 | DAP10 |
| CD137/41BB | BTNL3 | DAP12 |
| CD137/41BB | BTNL3 | CD32 |
| CD137/41BB | BTNL3 | CD79a |
| CD137/41BB | BTNL3 | CD79b |
| CD137/41BB | NKG2D | CD8 |
| CD137/41BB | NKG2D | CD3ζ |
| CD137/41BB | NKG2D | CD3δ |
| CD137/41BB | NKG2D | CD3γ |
| CD137/41BB | NKG2D | CD3ε |
| CD137/41BB | NKG2D | FcγRI-γ |
| CD137/41BB | NKG2D | FcγRIII-γ |
| CD137/41BB | NKG2D | FcεRIβ |
| CD137/41BB | NKG2D | FcεRIγ |
| CD137/41BB | NKG2D | DAP10 |
| CD137/41BB | NKG2D | DAP12 |
| CD137/41BB | NKG2D | CD32 |
| CD137/41BB | NKG2D | CD79a |
| CD137/41BB | NKG2D | CD79b |
| ICOS | CD28 | CD8 |
| ICOS | CD28 | CD3ζ |
| ICOS | CD28 | CD3δ |
| ICOS | CD28 | CD3γ |
| ICOS | CD28 | CD3ε |
| ICOS | CD28 | FcγRI-γ |
| ICOS | CD28 | FcγRIII-γ |
| ICOS | CD28 | FcεRIβ |
| ICOS | CD28 | FcεRIγ |
| ICOS | CD28 | DAP10 |
| ICOS | CD28 | DAP12 |
| ICOS | CD28 | CD32 |
| ICOS | CD28 | CD79a |
| ICOS | CD28 | CD79b |
| ICOS | CD8 | CD8 |
| ICOS | CD8 | CD3ζ |
| ICOS | CD8 | CD3δ |
| ICOS | CD8 | CD3γ |
| ICOS | CD8 | CD3ε |
| ICOS | CD8 | FcγRI-γ |
| ICOS | CD8 | FcγRIII-γ |
| ICOS | CD8 | FcεRIβ |
| ICOS | CD8 | FcεRIγ |
| ICOS | CD8 | DAP10 |
| ICOS | CD8 | DAP12 |
| ICOS | CD8 | CD32 |
| ICOS | CD8 | CD79a |
| ICOS | CD8 | CD79b |
| ICOS | CD4 | CD8 |
| ICOS | CD4 | CD3ζ |
| ICOS | CD4 | CD3δ |
| ICOS | CD4 | CD3γ |
| ICOS | CD4 | CD3ε |
| ICOS | CD4 | FcγRI-γ |
| ICOS | CD4 | FcγRIII-γ |
| ICOS | CD4 | FcεRIβ |
| ICOS | CD4 | FcεRIγ |
| ICOS | CD4 | DAP10 |
| ICOS | CD4 | DAP12 |
| ICOS | CD4 | CD32 |
| ICOS | CD4 | CD79a |
| ICOS | CD4 | CD79b |
| ICOS | b2c | CD8 |
| ICOS | b2c | CD3ζ |
| ICOS | b2c | CD3δ |
| ICOS | b2c | CD3γ |
| ICOS | b2c | CD3ε |
| ICOS | b2c | FcγRI-γ |
| ICOS | b2c | FcγRIII-γ |
| ICOS | b2c | FcεRIβ |
| ICOS | b2c | FcεRIγ |
| ICOS | b2c | DAP10 |
| ICOS | b2c | DAP12 |
| ICOS | b2c | CD32 |
| ICOS | b2c | CD79a |
| ICOS | b2c | CD79b |
| ICOS | CD137/41BB | CD8 |
| ICOS | CD137/41BB | CD3ζ |
| ICOS | CD137/41BB | CD3δ |
| ICOS | CD137/41BB | CD3γ |
| ICOS | CD137/41BB | CD3ε |
| ICOS | CD137/41BB | FcγRI-γ |
| ICOS | CD137/41BB | FcγRIII-γ |
| ICOS | CD137/41BB | FcεRIβ |
| ICOS | CD137/41BB | FcεRIγ |
| ICOS | CD137/41BB | DAP10 |
| ICOS | CD137/41BB | DAP12 |
| ICOS | CD137/41BB | CD32 |
| ICOS | CD137/41BB | CD79a |
| ICOS | CD137/41BB | CD79b |
| ICOS | ICOS | CD8 |
| ICOS | ICOS | CD3ζ |
| ICOS | ICOS | CD3δ |
| ICOS | ICOS | CD3γ |
| ICOS | ICOS | CD3ε |
| ICOS | ICOS | FcγRI-γ |
| ICOS | ICOS | FcγRIII-γ |
| ICOS | ICOS | FcεRIβ |
| ICOS | ICOS | FcεRIγ |
| ICOS | ICOS | DAP10 |
| ICOS | ICOS | DAP12 |
| ICOS | ICOS | CD32 |
| ICOS | ICOS | CD79a |
| ICOS | ICOS | CD79b |
| ICOS | CD27 | CD8 |
| ICOS | CD27 | CD3ζ |
| ICOS | CD27 | CD3δ |
| ICOS | CD27 | CD3γ |
| ICOS | CD27 | CD3ε |
| ICOS | CD27 | FcγRI-γ |
| ICOS | CD27 | FcγRIII-γ |
| ICOS | CD27 | FcεRIβ |
| ICOS | CD27 | FcεRIγ |
| ICOS | CD27 | DAP10 |
| ICOS | CD27 | DAP12 |
| ICOS | CD27 | CD32 |
| ICOS | CD27 | CD79a |
| ICOS | CD27 | CD79b |
| ICOS | CD28δ | CD8 |
| ICOS | CD28δ | CD3ζ |
| ICOS | CD28δ | CD3δ |
| ICOS | CD28δ | CD3γ |
| ICOS | CD28δ | CD3ε |
| ICOS | CD28δ | FcγRI-γ |
| ICOS | CD28δ | FcγRIII-γ |
| ICOS | CD28δ | FcεRIβ |
| ICOS | CD28δ | FcεRIγ |
| ICOS | CD28δ | DAP10 |
| ICOS | CD28δ | DAP12 |
| ICOS | CD28δ | CD32 |
| ICOS | CD28δ | CD79a |
| ICOS | CD28δ | CD79b |
| ICOS | CD80 | CD8 |
| ICOS | CD80 | CD3ζ |
| ICOS | CD80 | CD3δ |
| ICOS | CD80 | CD3γ |
| ICOS | CD80 | CD3ε |
| ICOS | CD80 | FcγRI-γ |
| ICOS | CD80 | FcγRIII-γ |
| ICOS | CD80 | FcεRIβ |
| ICOS | CD80 | FcεRIγ |
| ICOS | CD80 | DAP10 |
| ICOS | CD80 | DAP12 |
| ICOS | CD80 | CD32 |
| ICOS | CD80 | CD79a |
| ICOS | CD80 | CD79b |
| ICOS | CD86 | CD8 |
| ICOS | CD86 | CD3ζ |
| ICOS | CD86 | CD3δ |
| ICOS | CD86 | CD3γ |
| ICOS | CD86 | CD3ε |
| ICOS | CD86 | FcγRI-γ |
| ICOS | CD86 | FcγRIII-γ |
| ICOS | CD86 | FcεRIβ |
| ICOS | CD86 | FcεRIγ |
| ICOS | CD86 | DAP10 |
| ICOS | CD86 | DAP12 |
| ICOS | CD86 | CD32 |
| ICOS | CD86 | CD79a |
| ICOS | CD86 | CD79b |
| ICOS | OX40 | CD8 |
| ICOS | OX40 | CD3ζ |
| ICOS | OX40 | CD3δ |
| ICOS | OX40 | CD3γ |
| ICOS | OX40 | CD3ε |
| ICOS | OX40 | FcγRI-γ |
| ICOS | OX40 | FcγRIII-γ |
| ICOS | OX40 | FcεRIβ |
| ICOS | OX40 | FcεRIγ |
| ICOS | OX40 | DAP10 |
| ICOS | OX40 | DAP12 |
| ICOS | OX40 | CD32 |
| ICOS | OX40 | CD79a |
| ICOS | OX40 | CD79b |
| ICOS | DAP10 | CD8 |
| ICOS | DAP10 | CD3ζ |
| ICOS | DAP10 | CD3δ |
| ICOS | DAP10 | CD3γ |
| ICOS | DAP10 | CD3ε |
| ICOS | DAP10 | FcγRI-γ |
| ICOS | DAP10 | FcγRIII-γ |
| ICOS | DAP10 | FcεRIβ |
| ICOS | DAP10 | FcεRIγ |
| ICOS | DAP10 | DAP10 |
| ICOS | DAP10 | DAP12 |
| ICOS | DAP10 | CD32 |
| ICOS | DAP10 | CD79a |
| ICOS | DAP10 | CD79b |
| ICOS | DAP12 | CD8 |
| ICOS | DAP12 | CD3ζ |
| ICOS | DAP12 | CD3δ |
| ICOS | DAP12 | CD3γ |
| ICOS | DAP12 | CD3ε |
| ICOS | DAP12 | FcγRI-γ |
| ICOS | DAP12 | FcγRIII-γ |
| ICOS | DAP12 | FcεRIβ |
| ICOS | DAP12 | FcεRIγ |
| ICOS | DAP12 | DAP10 |
| ICOS | DAP12 | DAP12 |
| ICOS | DAP12 | CD32 |
| ICOS | DAP12 | CD79a |
| ICOS | DAP12 | CD79b |
| ICOS | MyD88 | CD8 |
| ICOS | MyD88 | CD3ζ |
| ICOS | MyD88 | CD3δ |
| ICOS | MyD88 | CD3γ |
| ICOS | MyD88 | CD3ε |
| ICOS | MyD88 | FcγRI-γ |
| ICOS | MyD88 | FcγRIII-γ |
| ICOS | MyD88 | FcεRIβ |
| ICOS | MyD88 | FcεRIγ |
| ICOS | MyD88 | DAP10 |
| ICOS | MyD88 | DAP12 |
| ICOS | MyD88 | CD32 |
| ICOS | MyD88 | CD79a |
| ICOS | MyD88 | CD79b |
| ICOS | CD7 | CD8 |
| ICOS | CD7 | CD3ζ |
| ICOS | CD7 | CD3δ |
| ICOS | CD7 | CD3γ |
| ICOS | CD7 | CD3ε |
| ICOS | CD7 | FcγRI-γ |
| ICOS | CD7 | FcγRIII-γ |
| ICOS | CD7 | FcεRIβ |
| ICOS | CD7 | FcεRIγ |
| ICOS | CD7 | DAP10 |
| ICOS | CD7 | DAP12 |
| ICOS | CD7 | CD32 |
| ICOS | CD7 | CD79a |
| ICOS | CD7 | CD79b |
| ICOS | BTNL3 | CD8 |
| ICOS | BTNL3 | CD3ζ |
| ICOS | BTNL3 | CD3δ |
| ICOS | BTNL3 | CD3γ |
| ICOS | BTNL3 | CD3ε |
| ICOS | BTNL3 | FcγRI-γ |
| ICOS | BTNL3 | FcγRIII-γ |
| ICOS | BTNL3 | FcεRIβ |
| ICOS | BTNL3 | FcεRIγ |
| ICOS | BTNL3 | DAP10 |
| ICOS | BTNL3 | DAP12 |
| ICOS | BTNL3 | CD32 |
| ICOS | BTNL3 | CD79a |
| ICOS | BTNL3 | CD79b |
| ICOS | NKG2D | CD8 |
| ICOS | NKG2D | CD3ζ |
| ICOS | NKG2D | CD3δ |
| ICOS | NKG2D | CD3γ |
| ICOS | NKG2D | CD3ε |
| ICOS | NKG2D | FcγRI-γ |
| ICOS | NKG2D | FcγRIII-γ |
| ICOS | NKG2D | FcεRIβ |
| ICOS | NKG2D | FcεRIγ |
| ICOS | NKG2D | DAP10 |
| ICOS | NKG2D | DAP12 |
| ICOS | NKG2D | CD32 |
| ICOS | NKG2D | CD79a |
| ICOS | NKG2D | CD79b |
| CD27 | CD28 | CD8 |
| CD27 | CD28 | CD3ζ |
| CD27 | CD28 | CD3δ |
| CD27 | CD28 | CD3γ |
| CD27 | CD28 | CD3ε |
| CD27 | CD28 | FcγRI-γ |
| CD27 | CD28 | FcγRIII-γ |
| CD27 | CD28 | FcεRIβ |
| CD27 | CD28 | FcεRIγ |
| CD27 | CD28 | DAP10 |
| CD27 | CD28 | DAP12 |
| CD27 | CD28 | CD32 |
| CD27 | CD28 | CD79a |
| CD27 | CD28 | CD79b |
| CD27 | CD8 | CD8 |
| CD27 | CD8 | CD3ζ |
| CD27 | CD8 | CD3δ |
| CD27 | CD8 | CD3γ |
| CD27 | CD8 | CD3ε |
| CD27 | CD8 | FcγRI-γ |
| CD27 | CD8 | FcγRIII-γ |
| CD27 | CD8 | FcεRIβ |
| CD27 | CD8 | FcεRIγ |
| CD27 | CD8 | DAP10 |
| CD27 | CD8 | DAP12 |
| CD27 | CD8 | CD32 |
| CD27 | CD8 | CD79a |
| CD27 | CD8 | CD79b |
| CD27 | CD4 | CD8 |
| CD27 | CD4 | CD3ζ |
| CD27 | CD4 | CD3δ |
| CD27 | CD4 | CD3γ |
| CD27 | CD4 | CD3ε |
| CD27 | CD4 | FcγRI-γ |
| CD27 | CD4 | FcγRIII-γ |
| CD27 | CD4 | FcεRIβ |
| CD27 | CD4 | FcεRIγ |
| CD27 | CD4 | DAP10 |
| CD27 | CD4 | DAP12 |
| CD27 | CD4 | CD32 |
| CD27 | CD4 | CD79a |
| CD27 | CD4 | CD79b |
| CD27 | b2c | CD8 |
| CD27 | b2c | CD3ζ |
| CD27 | b2c | CD3δ |
| CD27 | b2c | CD3γ |
| CD27 | b2c | CD3ε |
| CD27 | b2c | FcγRI-γ |
| CD27 | b2c | FcγRIII-γ |
| CD27 | b2c | FcεRIβ |
| CD27 | b2c | FcεRIγ |
| CD27 | b2c | DAP10 |
| CD27 | b2c | DAP12 |
| CD27 | b2c | CD32 |
| CD27 | b2c | CD79a |
| CD27 | b2c | CD79b |
| CD27 | CD137/41BB | CD8 |
| CD27 | CD137/41BB | CD3ζ |
| CD27 | CD137/41BB | CD3δ |
| CD27 | CD137/41BB | CD3γ |
| CD27 | CD137/41BB | CD3ε |
| CD27 | CD137/41BB | FcγRI-γ |
| CD27 | CD137/41BB | FcγRIII-γ |
| CD27 | CD137/41BB | FcεRIβ |
| CD27 | CD137/41BB | FcεRIγ |
| CD27 | CD137/41BB | DAP10 |
| CD27 | CD137/41BB | DAP12 |
| CD27 | CD137/41BB | CD32 |
| CD27 | CD137/41BB | CD79a |
| CD27 | CD137/41BB | CD79b |
| CD27 | ICOS | CD8 |
| CD27 | ICOS | CD3ζ |
| CD27 | ICOS | CD3δ |
| CD27 | ICOS | CD3γ |
| CD27 | ICOS | CD3ε |
| CD27 | ICOS | FcγRI-γ |
| CD27 | ICOS | FcγRIII-γ |
| CD27 | ICOS | FcεRIβ |
| CD27 | ICOS | FcεRIγ |
| CD27 | ICOS | DAP10 |
| CD27 | ICOS | DAP12 |
| CD27 | ICOS | CD32 |
| CD27 | ICOS | CD79a |
| CD27 | ICOS | CD79b |
| CD27 | CD27 | CD8 |
| CD27 | CD27 | CD3ζ |
| CD27 | CD27 | CD3δ |
| CD27 | CD27 | CD3γ |
| CD27 | CD27 | CD3ε |
| CD27 | CD27 | FcγRI-γ |
| CD27 | CD27 | FcγRIII-γ |
| CD27 | CD27 | FcεRIβ |
| CD27 | CD27 | FcεRIγ |
| CD27 | CD27 | DAP10 |
| CD27 | CD27 | DAP12 |
| CD27 | CD27 | CD32 |
| CD27 | CD27 | CD79a |
| CD27 | CD27 | CD79b |
| CD27 | CD28δ | CD8 |
| CD27 | CD28δ | CD3ζ |
| CD27 | CD28δ | CD3δ |
| CD27 | CD28δ | CD3γ |
| CD27 | CD28δ | CD3ε |
| CD27 | CD28δ | FcγRI-γ |
| CD27 | CD28δ | FcγRIII-γ |
| CD27 | CD28δ | FcεRIβ |
| CD27 | CD28δ | FcεRIγ |
| CD27 | CD28δ | DAP10 |
| CD27 | CD28δ | DAP12 |
| CD27 | CD28δ | CD32 |
| CD27 | CD28δ | CD79a |
| CD27 | CD28δ | CD79b |
| CD27 | CD80 | CD8 |
| CD27 | CD80 | CD3ζ |
| CD27 | CD80 | CD3δ |
| CD27 | CD80 | CD3γ |
| CD27 | CD80 | CD3ε |
| CD27 | CD80 | FcγRI-γ |
| CD27 | CD80 | FcγRIII-γ |
| CD27 | CD80 | FcεRIβ |
| CD27 | CD80 | FcεRIγ |
| CD27 | CD80 | DAP10 |
| CD27 | CD80 | DAP12 |
| CD27 | CD80 | CD32 |
| CD27 | CD80 | CD79a |
| CD27 | CD80 | CD79b |
| CD27 | CD86 | CD8 |
| CD27 | CD86 | CD3ζ |
| CD27 | CD86 | CD3δ |
| CD27 | CD86 | CD3γ |
| CD27 | CD86 | CD3ε |
| CD27 | CD86 | FcγRI-γ |
| CD27 | CD86 | FcγRIII-γ |
| CD27 | CD86 | FcεRIβ |
| CD27 | CD86 | FcεRIγ |
| CD27 | CD86 | DAP10 |
| CD27 | CD86 | DAP12 |
| CD27 | CD86 | CD32 |
| CD27 | CD86 | CD79a |
| CD27 | CD86 | CD79b |
| CD27 | OX40 | CD8 |
| CD27 | OX40 | CD3ζ |
| CD27 | OX40 | CD3δ |
| CD27 | OX40 | CD3γ |
| CD27 | OX40 | CD3ε |
| CD27 | OX40 | FcγRI-γ |
| CD27 | OX40 | FcγRIII-γ |
| CD27 | OX40 | FcεRIβ |
| CD27 | OX40 | FcεRIγ |
| CD27 | OX40 | DAP10 |
| CD27 | OX40 | DAP12 |
| CD27 | OX40 | CD32 |
| CD27 | OX40 | CD79a |
| CD27 | OX40 | CD79b |
| CD27 | DAP10 | CD8 |
| CD27 | DAP10 | CD3ζ |
| CD27 | DAP10 | CD3δ |
| CD27 | DAP10 | CD3γ |
| CD27 | DAP10 | CD3ε |
| CD27 | DAP10 | FcγRI-γ |
| CD27 | DAP10 | FcγRIII-γ |
| CD27 | DAP10 | FcεRIβ |
| CD27 | DAP10 | FcεRIγ |
| CD27 | DAP10 | DAP10 |
| CD27 | DAP10 | DAP12 |
| CD27 | DAP10 | CD32 |
| CD27 | DAP10 | CD79a |
| CD27 | DAP10 | CD79b |
| CD27 | DAP12 | CD8 |
| CD27 | DAP12 | CD3ζ |
| CD27 | DAP12 | CD3δ |
| CD27 | DAP12 | CD3γ |
| CD27 | DAP12 | CD3ε |
| CD27 | DAP12 | FcγRI-γ |
| CD27 | DAP12 | FcγRIII-γ |
| CD27 | DAP12 | FcεRIβ |
| CD27 | DAP12 | FcεRIγ |
| CD27 | DAP12 | DAP10 |
| CD27 | DAP12 | DAP12 |
| CD27 | DAP12 | CD32 |
| CD27 | DAP12 | CD79a |
| CD27 | DAP12 | CD79b |
| CD27 | MyD88 | CD8 |
| CD27 | MyD88 | CD3ζ |
| CD27 | MyD88 | CD3δ |
| CD27 | MyD88 | CD3γ |
| CD27 | MyD88 | CD3ε |
| CD27 | MyD88 | FcγRI-γ |
| CD27 | MyD88 | FcγRIII-γ |
| CD27 | MyD88 | FcεRIβ |
| CD27 | MyD88 | FcεRIγ |
| CD27 | MyD88 | DAP10 |
| CD27 | MyD88 | DAP12 |
| CD27 | MyD88 | CD32 |
| CD27 | MyD88 | CD79a |
| CD27 | MyD88 | CD79b |
| CD27 | CD7 | CD8 |
| CD27 | CD7 | CD3ζ |
| CD27 | CD7 | CD3δ |
| CD27 | CD7 | CD3γ |
| CD27 | CD7 | CD3ε |
| CD27 | CD7 | FcγRI-γ |
| CD27 | CD7 | FcγRIII-γ |
| CD27 | CD7 | FcεRIβ |
| CD27 | CD7 | FcεRIγ |
| CD27 | CD7 | DAP10 |
| CD27 | CD7 | DAP12 |
| CD27 | CD7 | CD32 |
| CD27 | CD7 | CD79a |
| CD27 | CD7 | CD79b |
| CD27 | BTNL3 | CD8 |
| CD27 | BTNL3 | CD3ζ |
| CD27 | BTNL3 | CD3δ |
| CD27 | BTNL3 | CD3γ |
| CD27 | BTNL3 | CD3ε |
| CD27 | BTNL3 | FcγRI-γ |
| CD27 | BTNL3 | FcγRIII-γ |
| CD27 | BTNL3 | FcεRIβ |
| CD27 | BTNL3 | FcεRIγ |
| CD27 | BTNL3 | DAP10 |
| CD27 | BTNL3 | DAP12 |
| CD27 | BTNL3 | CD32 |
| CD27 | BTNL3 | CD79a |
| CD27 | BTNL3 | CD79b |
| CD27 | NKG2D | CD8 |
| CD27 | NKG2D | CD3ζ |
| CD27 | NKG2D | CD3δ |
| CD27 | NKG2D | CD3γ |
| CD27 | NKG2D | CD3ε |
| CD27 | NKG2D | FcγRI-γ |
| CD27 | NKG2D | FcγRIII-γ |
| CD27 | NKG2D | FcεRIβ |
| CD27 | NKG2D | FcεRIγ |
| CD27 | NKG2D | DAP10 |
| CD27 | NKG2D | DAP12 |
| CD27 | NKG2D | CD32 |
| CD27 | NKG2D | CD79a |
| CD27 | NKG2D | CD79b |
| CD28δ | CD28 | CD8 |
| CD28δ | CD28 | CD3ζ |
| CD28δ | CD28 | CD3δ |
| CD28δ | CD28 | CD3γ |
| CD28δ | CD28 | CD3ε |
| CD28δ | CD28 | FcγRI-γ |
| CD28δ | CD28 | FcγRIII-γ |
| CD28δ | CD28 | FcεRIβ |
| CD28δ | CD28 | FcεRIγ |
| CD28δ | CD28 | DAP10 |
| CD28δ | CD28 | DAP12 |
| CD28δ | CD28 | CD32 |
| CD28δ | CD28 | CD79a |
| CD28δ | CD28 | CD79b |
| CD28δ | CD8 | CD8 |
| CD28δ | CD8 | CD3ζ |
| CD28δ | CD8 | CD3δ |
| CD28δ | CD8 | CD3γ |
| CD28δ | CD8 | CD3ε |
| CD28δ | CD8 | FcγRI-γ |
| CD28δ | CD8 | FcγRIII-γ |
| CD28δ | CD8 | FcεRIβ |
| CD28δ | CD8 | FcεRIγ |
| CD28δ | CD8 | DAP10 |
| CD28δ | CD8 | DAP12 |
| CD28δ | CD8 | CD32 |
| CD28δ | CD8 | CD79a |
| CD28δ | CD8 | CD79b |
| CD28δ | CD4 | CD8 |
| CD28δ | CD4 | CD3ζ |
| CD28δ | CD4 | CD3δ |
| CD28δ | CD4 | CD3γ |
| CD28δ | CD4 | CD3ε |
| CD28δ | CD4 | FcγRI-γ |
| CD28δ | CD4 | FcγRIII-γ |
| CD28δ | CD4 | FcεRIβ |
| CD28δ | CD4 | FcεRIγ |
| CD28δ | CD4 | DAP10 |
| CD28δ | CD4 | DAP12 |
| CD28δ | CD4 | CD32 |
| CD28δ | CD4 | CD79a |
| CD28δ | CD4 | CD79b |
| CD28δ | b2c | CD8 |
| CD28δ | b2c | CD3ζ |
| CD28δ | b2c | CD3δ |
| CD28δ | b2c | CD3γ |
| CD28δ | b2c | CD3ε |
| CD28δ | b2c | FcγRI-γ |
| CD28δ | b2c | FcγRIII-γ |
| CD28δ | b2c | FcεRIβ |
| CD28δ | b2c | FcεRIγ |
| CD28δ | b2c | DAP10 |
| CD28δ | b2c | DAP12 |
| CD28δ | b2c | CD32 |
| CD28δ | b2c | CD79a |
| CD28δ | b2c | CD79b |
| CD28δ | CD137/41BB | CD8 |
| CD28δ | CD137/41BB | CD3ζ |
| CD28δ | CD137/41BB | CD3δ |
| CD28δ | CD137/41BB | CD3γ |
| CD28δ | CD137/41BB | CD3ε |
| CD28δ | CD137/41BB | FcγRI-γ |
| CD28δ | CD137/41BB | FcγRIII-γ |
| CD28δ | CD137/41BB | FcεRIβ |
| CD28δ | CD137/41BB | FcεRIγ |
| CD28δ | CD137/41BB | DAP10 |
| CD28δ | CD137/41BB | DAP12 |
| CD28δ | CD137/41BB | CD32 |
| CD28δ | CD137/41BB | CD79a |
| CD28δ | CD137/41BB | CD79b |
| CD28δ | ICOS | CD8 |
| CD28δ | ICOS | CD3ζ |
| CD28δ | ICOS | CD3δ |
| CD28δ | ICOS | CD3γ |
| CD28δ | ICOS | CD3ε |
| CD28δ | ICOS | FcγRI-γ |
| CD28δ | ICOS | FcγRIII-γ |
| CD28δ | ICOS | FcεRIβ |
| CD28δ | ICOS | FcεRIγ |
| CD28δ | ICOS | DAP10 |
| CD28δ | ICOS | DAP12 |
| CD28δ | ICOS | CD32 |
| CD28δ | ICOS | CD79a |
| CD28δ | ICOS | CD79b |
| CD28δ | CD27 | CD8 |
| CD28δ | CD27 | CD3ζ |
| CD28δ | CD27 | CD3δ |
| CD28δ | CD27 | CD3γ |
| CD28δ | CD27 | CD3ε |
| CD28δ | CD27 | FcγRI-γ |
| CD28δ | CD27 | FcγRIII-γ |
| CD28δ | CD27 | FcεRIβ |
| CD28δ | CD27 | FcεRIγ |
| CD28δ | CD27 | DAP10 |
| CD28δ | CD27 | DAP12 |
| CD28δ | CD27 | CD32 |
| CD28δ | CD27 | CD79a |
| CD28δ | CD27 | CD79b |
| CD28δ | CD28δ | CD8 |
| CD28δ | CD28δ | CD3ζ |
| CD28δ | CD28δ | CD3δ |
| CD28δ | CD28δ | CD3γ |
| CD28δ | CD28δ | CD3ε |
| CD28δ | CD28δ | FcγRI-γ |
| CD28δ | CD28δ | FcγRIII-γ |
| CD28δ | CD28δ | FcεRIβ |
| CD28δ | CD28δ | FcεRIγ |
| CD28δ | CD28δ | DAP10 |
| CD28δ | CD28δ | DAP12 |
| CD28δ | CD28δ | CD32 |
| CD28δ | CD28δ | CD79a |
| CD28δ | CD28δ | CD79b |
| CD28δ | CD80 | CD8 |
| CD28δ | CD80 | CD3ζ |
| CD28δ | CD80 | CD3δ |
| CD28δ | CD80 | CD3γ |
| CD28δ | CD80 | CD3ε |
| CD28δ | CD80 | FcγRI-γ |
| CD28δ | CD80 | FcγRIII-γ |
| CD28δ | CD80 | FcεRIβ |
| CD28δ | CD80 | FcεRIγ |
| CD28δ | CD80 | DAP10 |
| CD28δ | CD80 | DAP12 |
| CD28δ | CD80 | CD32 |
| CD28δ | CD80 | CD79a |
| CD28δ | CD80 | CD79b |
| CD28δ | CD86 | CD8 |
| CD28δ | CD86 | CD3ζ |
| CD28δ | CD86 | CD3δ |
| CD28δ | CD86 | CD3γ |
| CD28δ | CD86 | CD3ε |
| CD28δ | CD86 | FcγRI-γ |
| CD28δ | CD86 | FcγRIII-γ |
| CD28δ | CD86 | FcεRIβ |
| CD28δ | CD86 | FcεRIγ |
| CD28δ | CD86 | DAP10 |
| CD28δ | CD86 | DAP12 |
| CD28δ | CD86 | CD32 |
| CD28δ | CD86 | CD79a |
| CD28δ | CD86 | CD79b |
| CD28δ | OX40 | CD8 |
| CD28δ | OX40 | CD3ζ |
| CD28δ | OX40 | CD3δ |
| CD28δ | OX40 | CD3γ |
| CD28δ | OX40 | CD3ε |
| CD28δ | OX40 | FcγRI-γ |
| CD28δ | OX40 | FcγRIII-γ |
| CD28δ | OX40 | FcεRIβ |
| CD28δ | OX40 | FcεRIγ |
| CD28δ | OX40 | DAP10 |
| CD28δ | OX40 | DAP12 |
| CD28δ | OX40 | CD32 |
| CD28δ | OX40 | CD79a |
| CD28δ | OX40 | CD79b |
| CD28δ | DAP10 | CD8 |
| CD28δ | DAP10 | CD3ζ |
| CD28δ | DAP10 | CD3δ |
| CD28δ | DAP10 | CD3γ |
| CD28δ | DAP10 | CD3ε |
| CD28δ | DAP10 | FcγRI-γ |
| CD28δ | DAP10 | FcγRIII-γ |
| CD28δ | DAP10 | FcεRIβ |
| CD28δ | DAP10 | FcεRIγ |
| CD28δ | DAP10 | DAP10 |
| CD28δ | DAP10 | DAP12 |
| CD28δ | DAP10 | CD32 |
| CD28δ | DAP10 | CD79a |
| CD28δ | DAP10 | CD79b |
| CD28δ | DAP12 | CD8 |
| CD28δ | DAP12 | CD3ζ |
| CD28δ | DAP12 | CD3δ |
| CD28δ | DAP12 | CD3γ |
| CD28δ | DAP12 | CD3ε |
| CD28δ | DAP12 | FcγRI-γ |
| CD28δ | DAP12 | FcγRIII-γ |
| CD28δ | DAP12 | FcεRIβ |
| CD28δ | DAP12 | FcεRIγ |
| CD28δ | DAP12 | DAP10 |
| CD28δ | DAP12 | DAP12 |
| CD28δ | DAP12 | CD32 |
| CD28δ | DAP12 | CD79a |
| CD28δ | DAP12 | CD79b |
| CD28δ | MyD88 | CD8 |
| CD28δ | MyD88 | CD3ζ |
| CD28δ | MyD88 | CD3δ |
| CD28δ | MyD88 | CD3γ |
| CD28δ | MyD88 | CD3ε |
| CD28δ | MyD88 | FcγRI-γ |
| CD28δ | MyD88 | FcγRIII-γ |
| CD28δ | MyD88 | FcεRIβ |
| CD28δ | MyD88 | FcεRIγ |
| CD28δ | MyD88 | DAP10 |
| CD28δ | MyD88 | DAP12 |
| CD28δ | MyD88 | CD32 |
| CD28δ | MyD88 | CD79a |
| CD28δ | MyD88 | CD79b |
| CD28δ | CD7 | CD8 |
| CD28δ | CD7 | CD3ζ |
| CD28δ | CD7 | CD3δ |
| CD28δ | CD7 | CD3γ |
| CD28δ | CD7 | CD3ε |
| CD28δ | CD7 | FcγRI-γ |
| CD28δ | CD7 | FcγRIII-γ |
| CD28δ | CD7 | FcεRIβ |
| CD28δ | CD7 | FcεRIγ |
| CD28δ | CD7 | DAP10 |
| CD28δ | CD7 | DAP12 |
| CD28δ | CD7 | CD32 |
| CD28δ | CD7 | CD79a |
| CD28δ | CD7 | CD79b |
| CD28δ | BTNL3 | CD8 |
| CD28δ | BTNL3 | CD3ζ |
| CD28δ | BTNL3 | CD3δ |
| CD28δ | BTNL3 | CD3γ |
| CD28δ | BTNL3 | CD3ε |
| CD28δ | BTNL3 | FcγRI-γ |
| CD28δ | BTNL3 | FcγRIII-γ |
| CD28δ | BTNL3 | FcεRIβ |
| CD28δ | BTNL3 | FcεRIγ |
| CD28δ | BTNL3 | DAP10 |
| CD28δ | BTNL3 | DAP12 |
| CD28δ | BTNL3 | CD32 |
| CD28δ | BTNL3 | CD79a |
| CD28δ | BTNL3 | CD79b |
| CD28δ | NKG2D | CD8 |
| CD28δ | NKG2D | CD3ζ |
| CD28δ | NKG2D | CD3δ |
| CD28δ | NKG2D | CD3γ |
| CD28δ | NKG2D | CD3ε |
| CD28δ | NKG2D | FcγRI-γ |
| CD28δ | NKG2D | FcγRIII-γ |
| CD28δ | NKG2D | FcεRIβ |
| CD28δ | NKG2D | FcεRIγ |
| CD28δ | NKG2D | DAP10 |
| CD28δ | NKG2D | DAP12 |
| CD28δ | NKG2D | CD32 |
| CD28δ | NKG2D | CD79a |
| CD28δ | NKG2D | CD79b |
| CD80 | CD28 | CD8 |
| CD80 | CD28 | CD3ζ |
| CD80 | CD28 | CD3δ |
| CD80 | CD28 | CD3γ |
| CD80 | CD28 | CD3ε |
| CD80 | CD28 | FcγRI-γ |
| CD80 | CD28 | FcγRIII-γ |
| CD80 | CD28 | FcεRIβ |
| CD80 | CD28 | FcεRIγ |
| CD80 | CD28 | DAP10 |
| CD80 | CD28 | DAP12 |
| CD80 | CD28 | CD32 |
| CD80 | CD28 | CD79a |
| CD80 | CD28 | CD79b |
| CD80 | CD8 | CD8 |
| CD80 | CD8 | CD3ζ |
| CD80 | CD8 | CD3δ |
| CD80 | CD8 | CD3γ |
| CD80 | CD8 | CD3ε |
| CD80 | CD8 | FcγRI-γ |
| CD80 | CD8 | FcγRIII-γ |
| CD80 | CD8 | FcεRIβ |
| CD80 | CD8 | FcεRIγ |
| CD80 | CD8 | DAP10 |
| CD80 | CD8 | DAP12 |
| CD80 | CD8 | CD32 |
| CD80 | CD8 | CD79a |
| CD80 | CD8 | CD79b |
| CD80 | CD4 | CD8 |
| CD80 | CD4 | CD3ζ |
| CD80 | CD4 | CD3δ |
| CD80 | CD4 | CD3γ |
| CD80 | CD4 | CD3ε |
| CD80 | CD4 | FcγRI-γ |
| CD80 | CD4 | FcγRIII-γ |
| CD80 | CD4 | FcεRIβ |
| CD80 | CD4 | FcεRIγ |
| CD80 | CD4 | DAP10 |
| CD80 | CD4 | DAP12 |
| CD80 | CD4 | CD32 |
| CD80 | CD4 | CD79a |
| CD80 | CD4 | CD79b |
| CD80 | b2c | CD8 |
| CD80 | b2c | CD3ζ |
| CD80 | b2c | CD3δ |
| CD80 | b2c | CD3γ |
| CD80 | b2c | CD3ε |
| CD80 | b2c | FcγRI-γ |
| CD80 | b2c | FcγRIII-γ |
| CD80 | b2c | FcεRIβ |
| CD80 | b2c | FcεRIγ |
| CD80 | b2c | DAP10 |
| CD80 | b2c | DAP12 |
| CD80 | b2c | CD32 |
| CD80 | b2c | CD79a |
| CD80 | b2c | CD79b |
| CD80 | CD137/41BB | CD8 |
| CD80 | CD137/41BB | CD3ζ |
| CD80 | CD137/41BB | CD3δ |
| CD80 | CD137/41BB | CD3γ |
| CD80 | CD137/41BB | CD3ε |
| CD80 | CD137/41BB | FcγRI-γ |
| CD80 | CD137/41BB | FcγRIII-γ |
| CD80 | CD137/41BB | FcεRIβ |
| CD80 | CD137/41BB | FcεRIγ |
| CD80 | CD137/41BB | DAP10 |
| CD80 | CD137/41BB | DAP12 |
| CD80 | CD137/41BB | CD32 |
| CD80 | CD137/41BB | CD79a |
| CD80 | CD137/41BB | CD79b |
| CD80 | ICOS | CD8 |
| CD80 | ICOS | CD3ζ |
| CD80 | ICOS | CD3δ |
| CD80 | ICOS | CD3γ |
| CD80 | ICOS | CD3ε |
| CD80 | ICOS | FcγRI-γ |
| CD80 | ICOS | FcγRIII-γ |
| CD80 | ICOS | FcεRIβ |
| CD80 | ICOS | FcεRIγ |
| CD80 | ICOS | DAP10 |
| CD80 | ICOS | DAP12 |
| CD80 | ICOS | CD32 |
| CD80 | ICOS | CD79a |
| CD80 | ICOS | CD79b |
| CD80 | CD27 | CD8 |
| CD80 | CD27 | CD3ζ |
| CD80 | CD27 | CD3δ |
| CD80 | CD27 | CD3γ |
| CD80 | CD27 | CD3ε |
| CD80 | CD27 | FcγRI-γ |
| CD80 | CD27 | FcγRIII-γ |
| CD80 | CD27 | FcεRIβ |
| CD80 | CD27 | FcεRIγ |
| CD80 | CD27 | DAP10 |
| CD80 | CD27 | DAP12 |
| CD80 | CD27 | CD32 |
| CD80 | CD27 | CD79a |
| CD80 | CD27 | CD79b |
| CD80 | CD28δ | CD8 |
| CD80 | CD28δ | CD3ζ |
| CD80 | CD28δ | CD3δ |
| CD80 | CD28δ | CD3γ |
| CD80 | CD28δ | CD3ε |
| CD80 | CD28δ | FcγRI-γ |
| CD80 | CD28δ | FcγRIII-γ |
| CD80 | CD28δ | FcεRIβ |
| CD80 | CD28δ | FcεRIγ |
| CD80 | CD28δ | DAP10 |
| CD80 | CD28δ | DAP12 |
| CD80 | CD28δ | CD32 |
| CD80 | CD28δ | CD79a |
| CD80 | CD28δ | CD79b |
| CD80 | CD80 | CD8 |
| CD80 | CD80 | CD3ζ |
| CD80 | CD80 | CD3δ |
| CD80 | CD80 | CD3γ |
| CD80 | CD80 | CD3ε |
| CD80 | CD80 | FcγRI-γ |
| CD80 | CD80 | FcγRIII-γ |
| CD80 | CD80 | FcεRIβ |
| CD80 | CD80 | FcεRIγ |
| CD80 | CD80 | DAP10 |
| CD80 | CD80 | DAP12 |
| CD80 | CD80 | CD32 |
| CD80 | CD80 | CD79a |
| CD80 | CD80 | CD79b |
| CD80 | CD86 | CD8 |
| CD80 | CD86 | CD3ζ |
| CD80 | CD86 | CD3δ |
| CD80 | CD86 | CD3γ |
| CD80 | CD86 | CD3ε |
| CD80 | CD86 | FcγRI-γ |
| CD80 | CD86 | FcγRIII-γ |
| CD80 | CD86 | FcεRIβ |
| CD80 | CD86 | FcεRIγ |
| CD80 | CD86 | DAP10 |
| CD80 | CD86 | DAP12 |
| CD80 | CD86 | CD32 |
| CD80 | CD86 | CD79a |
| CD80 | CD86 | CD79b |
| CD80 | OX40 | CD8 |
| CD80 | OX40 | CD3ζ |
| CD80 | OX40 | CD3δ |
| CD80 | OX40 | CD3γ |
| CD80 | OX40 | CD3ε |
| CD80 | OX40 | FcγRI-γ |
| CD80 | OX40 | FcγRIII-γ |
| CD80 | OX40 | FcεRIβ |
| CD80 | OX40 | FcεRIγ |
| CD80 | OX40 | DAP10 |
| CD80 | OX40 | DAP12 |
| CD80 | OX40 | CD32 |
| CD80 | OX40 | CD79a |
| CD80 | OX40 | CD79b |
| CD80 | DAP10 | CD8 |
| CD80 | DAP10 | CD3ζ |
| CD80 | DAP10 | CD3δ |
| CD80 | DAP10 | CD3γ |
| CD80 | DAP10 | CD3ε |
| CD80 | DAP10 | FcγRI-γ |
| CD80 | DAP10 | FcγRIII-γ |
| CD80 | DAP10 | FcεRIβ |
| CD80 | DAP10 | FcεRIγ |
| CD80 | DAP10 | DAP10 |
| CD80 | DAP10 | DAP12 |
| CD80 | DAP10 | CD32 |
| CD80 | DAP10 | CD79a |
| CD80 | DAP10 | CD79b |
| CD80 | DAP12 | CD8 |
| CD80 | DAP12 | CD3ζ |
| CD80 | DAP12 | CD3δ |
| CD80 | DAP12 | CD3γ |
| CD80 | DAP12 | CD3ε |
| CD80 | DAP12 | FcγRI-γ |
| CD80 | DAP12 | FcγRIII-γ |
| CD80 | DAP12 | FcεRIβ |
| CD80 | DAP12 | FcεRIγ |
| CD80 | DAP12 | DAP10 |
| CD80 | DAP12 | DAP12 |
| CD80 | DAP12 | CD32 |
| CD80 | DAP12 | CD79a |
| CD80 | DAP12 | CD79b |
| CD80 | MyD88 | CD8 |
| CD80 | MyD88 | CD3ζ |
| CD80 | MyD88 | CD3δ |
| CD80 | MyD88 | CD3γ |
| CD80 | MyD88 | CD3ε |
| CD80 | MyD88 | FcγRI-γ |
| CD80 | MyD88 | FcγRIII-γ |
| CD80 | MyD88 | FcεRIβ |
| CD80 | MyD88 | FcεRIγ |
| CD80 | MyD88 | DAP10 |
| CD80 | MyD88 | DAP12 |
| CD80 | MyD88 | CD32 |
| CD80 | MyD88 | CD79a |
| CD80 | MyD88 | CD79b |
| CD80 | CD7 | CD8 |
| CD80 | CD7 | CD3ζ |
| CD80 | CD7 | CD3δ |
| CD80 | CD7 | CD3γ |
| CD80 | CD7 | CD3ε |
| CD80 | CD7 | FcγRI-γ |
| CD80 | CD7 | FcγRIII-γ |
| CD80 | CD7 | FcεRIβ |
| CD80 | CD7 | FcεRIγ |
| CD80 | CD7 | DAP10 |
| CD80 | CD7 | DAP12 |
| CD80 | CD7 | CD32 |
| CD80 | CD7 | CD79a |
| CD80 | CD7 | CD79b |
| CD80 | BTNL3 | CD8 |
| CD80 | BTNL3 | CD3ζ |
| CD80 | BTNL3 | CD3δ |
| CD80 | BTNL3 | CD3γ |
| CD80 | BTNL3 | CD3ε |
| CD80 | BTNL3 | FcγRI-γ |
| CD80 | BTNL3 | FcγRIII-γ |
| CD80 | BTNL3 | FcεRIβ |
| CD80 | BTNL3 | FcεRIγ |
| CD80 | BTNL3 | DAP10 |
| CD80 | BTNL3 | DAP12 |
| CD80 | BTNL3 | CD32 |
| CD80 | BTNL3 | CD79a |
| CD80 | BTNL3 | CD79b |
| CD80 | NKG2D | CD8 |
| CD80 | NKG2D | CD3ζ |
| CD80 | NKG2D | CD3δ |
| CD80 | NKG2D | CD3γ |
| CD80 | NKG2D | CD3ε |
| CD80 | NKG2D | FcγRI-γ |
| CD80 | NKG2D | FcγRIII-γ |
| CD80 | NKG2D | FcεRIβ |
| CD80 | NKG2D | FcεRIγ |
| CD80 | NKG2D | DAP10 |
| CD80 | NKG2D | DAP12 |
| CD80 | NKG2D | CD32 |
| CD80 | NKG2D | CD79a |
| CD80 | NKG2D | CD79b |
| CD86 | CD28 | CD8 |
| CD86 | CD28 | CD3ζ |
| CD86 | CD28 | CD3δ |
| CD86 | CD28 | CD3γ |
| CD86 | CD28 | CD3ε |
| CD86 | CD28 | FcγRI-γ |
| CD86 | CD28 | FcγRIII-γ |
| CD86 | CD28 | FcεRIβ |
| CD86 | CD28 | FcεRIγ |
| CD86 | CD28 | DAP10 |
| CD86 | CD28 | DAP12 |
| CD86 | CD28 | CD32 |
| CD86 | CD28 | CD79a |
| CD86 | CD28 | CD79b |
| CD86 | CD8 | CD8 |
| CD86 | CD8 | CD3ζ |
| CD86 | CD8 | CD3δ |
| CD86 | CD8 | CD3γ |
| CD86 | CD8 | CD3ε |
| CD86 | CD8 | FcγRI-γ |
| CD86 | CD8 | FcγRIII-γ |
| CD86 | CD8 | FcεRIβ |
| CD86 | CD8 | FcεRIγ |
| CD86 | CD8 | DAP10 |
| CD86 | CD8 | DAP12 |
| CD86 | CD8 | CD32 |
| CD86 | CD8 | CD79a |
| CD86 | CD8 | CD79b |
| CD86 | CD4 | CD8 |
| CD86 | CD4 | CD3ζ |
| CD86 | CD4 | CD3δ |
| CD86 | CD4 | CD3γ |
| CD86 | CD4 | CD3ε |
| CD86 | CD4 | FcγRI-γ |
| CD86 | CD4 | FcγRIII-γ |
| CD86 | CD4 | FcεRIβ |
| CD86 | CD4 | FcεRIγ |
| CD86 | CD4 | DAP10 |
| CD86 | CD4 | DAP12 |
| CD86 | CD4 | CD32 |
| CD86 | CD4 | CD79a |
| CD86 | CD4 | CD79b |
| CD86 | b2c | CD8 |
| CD86 | b2c | CD3ζ |
| CD86 | b2c | CD3δ |
| CD86 | b2c | CD3γ |
| CD86 | b2c | CD3ε |
| CD86 | b2c | FcγRI-γ |
| CD86 | b2c | FcγRIII-γ |
| CD86 | b2c | FcεRIβ |
| CD86 | b2c | FcεRIγ |
| CD86 | b2c | DAP10 |
| CD86 | b2c | DAP12 |
| CD86 | b2c | CD32 |
| CD86 | b2c | CD79a |
| CD86 | b2c | CD79b |
| CD86 | CD137/41BB | CD8 |
| CD86 | CD137/41BB | CD3ζ |
| CD86 | CD137/41BB | CD3δ |
| CD86 | CD137/41BB | CD3γ |
| CD86 | CD137/41BB | CD3ε |
| CD86 | CD137/41BB | FcγRI-γ |
| CD86 | CD137/41BB | FcγRIII-γ |
| CD86 | CD137/41BB | FcεRIβ |
| CD86 | CD137/41BB | FcεRIγ |
| CD86 | CD137/41BB | DAP10 |
| CD86 | CD137/41BB | DAP12 |
| CD86 | CD137/41BB | CD32 |
| CD86 | CD137/41BB | CD79a |
| CD86 | CD137/41BB | CD79b |
| CD86 | ICOS | CD8 |
| CD86 | ICOS | CD3ζ |
| CD86 | ICOS | CD3δ |
| CD86 | ICOS | CD3γ |
| CD86 | ICOS | CD3ε |
| CD86 | ICOS | FcγRI-γ |
| CD86 | ICOS | FcγRIII-γ |
| CD86 | ICOS | FcεRIβ |
| CD86 | ICOS | FcεRIγ |
| CD86 | ICOS | DAP10 |
| CD86 | ICOS | DAP12 |
| CD86 | ICOS | CD32 |
| CD86 | ICOS | CD79a |
| CD86 | ICOS | CD79b |
| CD86 | CD27 | CD8 |
| CD86 | CD27 | CD3ζ |
| CD86 | CD27 | CD3δ |
| CD86 | CD27 | CD3γ |
| CD86 | CD27 | CD3ε |
| CD86 | CD27 | FcγRI-γ |
| CD86 | CD27 | FcγRIII-γ |
| CD86 | CD27 | FcεRIβ |
| CD86 | CD27 | FcεRIγ |
| CD86 | CD27 | DAP10 |
| CD86 | CD27 | DAP12 |
| CD86 | CD27 | CD32 |
| CD86 | CD27 | CD79a |
| CD86 | CD27 | CD79b |
| CD86 | CD28δ | CD8 |
| CD86 | CD28δ | CD3ζ |
| CD86 | CD28δ | CD3δ |
| CD86 | CD28δ | CD3γ |
| CD86 | CD28δ | CD3ε |
| CD86 | CD28δ | FcγRI-γ |
| CD86 | CD28δ | FcγRIII-γ |
| CD86 | CD28δ | FcεRIβ |
| CD86 | CD28δ | FcεRIγ |
| CD86 | CD28δ | DAP10 |
| CD86 | CD28δ | DAP12 |
| CD86 | CD28δ | CD32 |
| CD86 | CD28δ | CD79a |
| CD86 | CD28δ | CD79b |
| CD86 | CD80 | CD8 |
| CD86 | CD80 | CD3ζ |
| CD86 | CD80 | CD3δ |
| CD86 | CD80 | CD3γ |
| CD86 | CD80 | CD3ε |
| CD86 | CD80 | FcγRI-γ |
| CD86 | CD80 | FcγRIII-γ |
| CD86 | CD80 | FcεRIβ |
| CD86 | CD80 | FcεRIγ |
| CD86 | CD80 | DAP10 |
| CD86 | CD80 | DAP12 |
| CD86 | CD80 | CD32 |
| CD86 | CD80 | CD79a |
| CD86 | CD80 | CD79b |
| CD86 | CD86 | CD8 |
| CD86 | CD86 | CD3ζ |
| CD86 | CD86 | CD3δ |
| CD86 | CD86 | CD3γ |
| CD86 | CD86 | CD3ε |
| CD86 | CD86 | FcγRI-γ |
| CD86 | CD86 | FcγRIII-γ |
| CD86 | CD86 | FcεRIβ |
| CD86 | CD86 | FcεRIγ |
| CD86 | CD86 | DAP10 |
| CD86 | CD86 | DAP12 |
| CD86 | CD86 | CD32 |
| CD86 | CD86 | CD79a |
| CD86 | CD86 | CD79b |
| CD86 | OX40 | CD8 |
| CD86 | OX40 | CD3ζ |
| CD86 | OX40 | CD3δ |
| CD86 | OX40 | CD3γ |
| CD86 | OX40 | CD3ε |
| CD86 | OX40 | FcγRI-γ |
| CD86 | OX40 | FcγRIII-γ |
| CD86 | OX40 | FcεRIβ |
| CD86 | OX40 | FcεRIγ |
| CD86 | OX40 | DAP10 |
| CD86 | OX40 | DAP12 |
| CD86 | OX40 | CD32 |
| CD86 | OX40 | CD79a |
| CD86 | OX40 | CD79b |
| CD86 | DAP10 | CD8 |
| CD86 | DAP10 | CD3ζ |
| CD86 | DAP10 | CD3δ |
| CD86 | DAP10 | CD3γ |
| CD86 | DAP10 | CD3ε |
| CD86 | DAP10 | FcγRI-γ |
| CD86 | DAP10 | FcγRIII-γ |
| CD86 | DAP10 | FcεRIβ |
| CD86 | DAP10 | FcεRIγ |
| CD86 | DAP10 | DAP10 |
| CD86 | DAP10 | DAP12 |
| CD86 | DAP10 | CD32 |
| CD86 | DAP10 | CD79a |
| CD86 | DAP10 | CD79b |
| CD86 | DAP12 | CD8 |
| CD86 | DAP12 | CD3ζ |
| CD86 | DAP12 | CD3δ |
| CD86 | DAP12 | CD3γ |
| CD86 | DAP12 | CD3ε |
| CD86 | DAP12 | FcγRI-γ |
| CD86 | DAP12 | FcγRIII-γ |
| CD86 | DAP12 | FcεRIβ |
| CD86 | DAP12 | FcεRIγ |
| CD86 | DAP12 | DAP10 |
| CD86 | DAP12 | DAP12 |
| CD86 | DAP12 | CD32 |
| CD86 | DAP12 | CD79a |
| CD86 | DAP12 | CD79b |
| CD86 | MyD88 | CD8 |
| CD86 | MyD88 | CD3ζ |
| CD86 | MyD88 | CD3δ |
| CD86 | MyD88 | CD3γ |
| CD86 | MyD88 | CD3ε |
| CD86 | MyD88 | FcγRI-γ |
| CD86 | MyD88 | FcγRIII-γ |
| CD86 | MyD88 | FcεRIβ |
| CD86 | MyD88 | FcεRIγ |
| CD86 | MyD88 | DAP10 |
| CD86 | MyD88 | DAP12 |
| CD86 | MyD88 | CD32 |
| CD86 | MyD88 | CD79a |
| CD86 | MyD88 | CD79b |
| CD86 | CD7 | CD8 |
| CD86 | CD7 | CD3ζ |
| CD86 | CD7 | CD3δ |
| CD86 | CD7 | CD3γ |
| CD86 | CD7 | CD3ε |
| CD86 | CD7 | FcγRI-γ |
| CD86 | CD7 | FcγRIII-γ |
| CD86 | CD7 | FcεRIβ |
| CD86 | CD7 | FcεRIγ |
| CD86 | CD7 | DAP10 |
| CD86 | CD7 | DAP12 |
| CD86 | CD7 | CD32 |
| CD86 | CD7 | CD79a |
| CD86 | CD7 | CD79b |
| CD86 | BTNL3 | CD8 |
| CD86 | BTNL3 | CD3ζ |
| CD86 | BTNL3 | CD3δ |
| CD86 | BTNL3 | CD3γ |
| CD86 | BTNL3 | CD3ε |
| CD86 | BTNL3 | FcγRI-γ |
| CD86 | BTNL3 | FcγRIII-γ |
| CD86 | BTNL3 | FcεRIβ |
| CD86 | BTNL3 | FcεRIγ |
| CD86 | BTNL3 | DAP10 |
| CD86 | BTNL3 | DAP12 |
| CD86 | BTNL3 | CD32 |
| CD86 | BTNL3 | CD79a |
| CD86 | BTNL3 | CD79b |
| CD86 | NKG2D | CD8 |
| CD86 | NKG2D | CD3ζ |
| CD86 | NKG2D | CD3δ |
| CD86 | NKG2D | CD3γ |
| CD86 | NKG2D | CD3ε |
| CD86 | NKG2D | FcγRI-γ |
| CD86 | NKG2D | FcγRIII-γ |
| CD86 | NKG2D | FcεRIβ |
| CD86 | NKG2D | FcεRIγ |
| CD86 | NKG2D | DAP10 |
| CD86 | NKG2D | DAP12 |
| CD86 | NKG2D | CD32 |
| CD86 | NKG2D | CD79a |
| CD86 | NKG2D | CD79b |
| OX40 | CD28 | CD8 |
| OX40 | CD28 | CD3ζ |
| OX40 | CD28 | CD3δ |
| OX40 | CD28 | CD3γ |
| OX40 | CD28 | CD3ε |
| OX40 | CD28 | FcγRI-γ |
| OX40 | CD28 | FcγRIII-γ |
| OX40 | CD28 | FcεRIβ |
| OX40 | CD28 | FcεRIγ |
| OX40 | CD28 | DAP10 |
| OX40 | CD28 | DAP12 |
| OX40 | CD28 | CD32 |
| OX40 | CD28 | CD79a |
| OX40 | CD28 | CD79b |
| OX40 | CD8 | CD8 |
| OX40 | CD8 | CD3ζ |
| OX40 | CD8 | CD3δ |
| OX40 | CD8 | CD3γ |
| OX40 | CD8 | CD3ε |
| OX40 | CD8 | FcγRI-γ |
| OX40 | CD8 | FcγRIII-γ |
| OX40 | CD8 | FcεRIβ |
| OX40 | CD8 | FcεRIγ |
| OX40 | CD8 | DAP10 |
| OX40 | CD8 | DAP12 |
| OX40 | CD8 | CD32 |
| OX40 | CD8 | CD79a |
| OX40 | CD8 | CD79b |
| OX40 | CD4 | CD8 |
| OX40 | CD4 | CD3ζ |
| OX40 | CD4 | CD3δ |
| OX40 | CD4 | CD3γ |
| OX40 | CD4 | CD3ε |
| OX40 | CD4 | FcγRI-γ |
| OX40 | CD4 | FcγRIII-γ |
| OX40 | CD4 | FcεRIβ |
| OX40 | CD4 | FcεRIγ |
| OX40 | CD4 | DAP10 |
| OX40 | CD4 | DAP12 |
| OX40 | CD4 | CD32 |
| OX40 | CD4 | CD79a |
| OX40 | CD4 | CD79b |
| OX40 | b2c | CD8 |
| OX40 | b2c | CD3ζ |
| OX40 | b2c | CD3δ |
| OX40 | b2c | CD3γ |
| OX40 | b2c | CD3ε |
| OX40 | b2c | FcγRI-γ |
| OX40 | b2c | FcγRIII-γ |
| OX40 | b2c | FcεRIβ |
| OX40 | b2c | FcεRIγ |
| OX40 | b2c | DAP10 |
| OX40 | b2c | DAP12 |
| OX40 | b2c | CD32 |
| OX40 | b2c | CD79a |
| OX40 | b2c | CD79b |
| OX40 | CD137/41BB | CD8 |
| OX40 | CD137/41BB | CD3ζ |
| OX40 | CD137/41BB | CD3δ |
| OX40 | CD137/41BB | CD3γ |
| OX40 | CD137/41BB | CD3ε |
| OX40 | CD137/41BB | FcγRI-γ |
| OX40 | CD137/41BB | FcγRIII-γ |
| OX40 | CD137/41BB | FcεRIβ |
| OX40 | CD137/41BB | FcεRIγ |
| OX40 | CD137/41BB | DAP10 |
| OX40 | CD137/41BB | DAP12 |
| OX40 | CD137/41BB | CD32 |
| OX40 | CD137/41BB | CD79a |
| OX40 | CD137/41BB | CD79b |
| OX40 | ICOS | CD8 |
| OX40 | ICOS | CD3ζ |
| OX40 | ICOS | CD3δ |
| OX40 | ICOS | CD3γ |
| OX40 | ICOS | CD3ε |
| OX40 | ICOS | FcγRI-γ |
| OX40 | ICOS | FcγRIII-γ |
| OX40 | ICOS | FcεRIβ |
| OX40 | ICOS | FcεRIγ |
| OX40 | ICOS | DAP10 |
| OX40 | ICOS | DAP12 |
| OX40 | ICOS | CD32 |
| OX40 | ICOS | CD79a |
| OX40 | ICOS | CD79b |
| OX40 | CD27 | CD8 |
| OX40 | CD27 | CD3ζ |
| OX40 | CD27 | CD3δ |
| OX40 | CD27 | CD3γ |
| OX40 | CD27 | CD3ε |
| OX40 | CD27 | FcγRI-γ |
| OX40 | CD27 | FcγRIII-γ |
| OX40 | CD27 | FcεRIβ |
| OX40 | CD27 | FcεRIγ |
| OX40 | CD27 | DAP10 |
| OX40 | CD27 | DAP12 |
| OX40 | CD27 | CD32 |
| OX40 | CD27 | CD79a |
| OX40 | CD27 | CD79b |
| OX40 | CD28δ | CD8 |
| OX40 | CD28δ | CD3ζ |
| OX40 | CD28δ | CD3δ |
| OX40 | CD28δ | CD3γ |
| OX40 | CD28δ | CD3ε |
| OX40 | CD28δ | FcγRI-γ |
| OX40 | CD28δ | FcγRIII-γ |
| OX40 | CD28δ | FcεRIβ |
| OX40 | CD28δ | FcεRIγ |
| OX40 | CD28δ | DAP10 |
| OX40 | CD28δ | DAP12 |
| OX40 | CD28δ | CD32 |
| OX40 | CD28δ | CD79a |
| OX40 | CD28δ | CD79b |
| OX40 | CD80 | CD8 |
| OX40 | CD80 | CD3ζ |
| OX40 | CD80 | CD3δ |
| OX40 | CD80 | CD3γ |
| OX40 | CD80 | CD3ε |
| OX40 | CD80 | FcγRI-γ |
| OX40 | CD80 | FcγRIII-γ |
| OX40 | CD80 | FcεRIβ |
| OX40 | CD80 | FcεRIγ |
| OX40 | CD80 | DAP10 |
| OX40 | CD80 | DAP12 |
| OX40 | CD80 | CD32 |
| OX40 | CD80 | CD79a |
| OX40 | CD80 | CD79b |
| OX40 | CD86 | CD8 |
| OX40 | CD86 | CD3ζ |
| OX40 | CD86 | CD3δ |
| OX40 | CD86 | CD3γ |
| OX40 | CD86 | CD3ε |
| OX40 | CD86 | FcγRI-γ |
| OX40 | CD86 | FcγRIII-γ |
| OX40 | CD86 | FcεRIβ |
| OX40 | CD86 | FcεRIγ |
| OX40 | CD86 | DAP10 |
| OX40 | CD86 | DAP12 |
| OX40 | CD86 | CD32 |
| OX40 | CD86 | CD79a |
| OX40 | CD86 | CD79b |
| OX40 | OX40 | CD8 |
| OX40 | OX40 | CD3ζ |
| OX40 | OX40 | CD3δ |
| OX40 | OX40 | CD3γ |
| OX40 | OX40 | CD3ε |
| OX40 | OX40 | FcγRI-γ |
| OX40 | OX40 | FcγRIII-γ |
| OX40 | OX40 | FcεRIβ |
| OX40 | OX40 | FcεRIγ |
| OX40 | OX40 | DAP10 |
| OX40 | OX40 | DAP12 |
| OX40 | OX40 | CD32 |
| OX40 | OX40 | CD79a |
| OX40 | OX40 | CD79b |
| OX40 | DAP10 | CD8 |
| OX40 | DAP10 | CD3ζ |
| OX40 | DAP10 | CD3δ |
| OX40 | DAP10 | CD3γ |
| OX40 | DAP10 | CD3ε |
| OX40 | DAP10 | FcγRI-γ |
| OX40 | DAP10 | FcγRIII-γ |
| OX40 | DAP10 | FcεRIβ |
| OX40 | DAP10 | FcεRIγ |
| OX40 | DAP10 | DAP10 |
| OX40 | DAP10 | DAP12 |
| OX40 | DAP10 | CD32 |
| OX40 | DAP10 | CD79a |
| OX40 | DAP10 | CD79b |
| OX40 | DAP12 | CD8 |
| OX40 | DAP12 | CD3ζ |
| OX40 | DAP12 | CD3δ |
| OX40 | DAP12 | CD3γ |
| OX40 | DAP12 | CD3ε |
| OX40 | DAP12 | FcγRI-γ |
| OX40 | DAP12 | FcγRIII-γ |
| OX40 | DAP12 | FcεRIβ |
| OX40 | DAP12 | FcεRIγ |
| OX40 | DAP12 | DAP10 |
| OX40 | DAP12 | DAP12 |
| OX40 | DAP12 | CD32 |
| OX40 | DAP12 | CD79a |
| OX40 | DAP12 | CD79b |
| OX40 | MyD88 | CD8 |
| OX40 | MyD88 | CD3ζ |
| OX40 | MyD88 | CD3δ |
| OX40 | MyD88 | CD3γ |
| OX40 | MyD88 | CD3ε |
| OX40 | MyD88 | FcγRI-γ |
| OX40 | MyD88 | FcγRIII-γ |
| OX40 | MyD88 | FcεRIβ |
| OX40 | MyD88 | FcεRIγ |
| OX40 | MyD88 | DAP10 |
| OX40 | MyD88 | DAP12 |
| OX40 | MyD88 | CD32 |
| OX40 | MyD88 | CD79a |
| OX40 | MyD88 | CD79b |
| OX40 | CD7 | CD8 |
| OX40 | CD7 | CD3ζ |
| OX40 | CD7 | CD3δ |
| OX40 | CD7 | CD3γ |
| OX40 | CD7 | CD3ε |
| OX40 | CD7 | FcγRI-γ |
| OX40 | CD7 | FcγRIII-γ |
| OX40 | CD7 | FcεRIβ |
| OX40 | CD7 | FcεRIγ |
| OX40 | CD7 | DAP10 |
| OX40 | CD7 | DAP12 |
| OX40 | CD7 | CD32 |
| OX40 | CD7 | CD79a |
| OX40 | CD7 | CD79b |
| OX40 | BTNL3 | CD8 |
| OX40 | BTNL3 | CD3ζ |
| OX40 | BTNL3 | CD3δ |
| OX40 | BTNL3 | CD3γ |
| OX40 | BTNL3 | CD3ε |
| OX40 | BTNL3 | FcγRI-γ |
| OX40 | BTNL3 | FcγRIII-γ |
| OX40 | BTNL3 | FcεRIβ |
| OX40 | BTNL3 | FcεRIγ |
| OX40 | BTNL3 | DAP10 |
| OX40 | BTNL3 | DAP12 |
| OX40 | BTNL3 | CD32 |
| OX40 | BTNL3 | CD79a |
| OX40 | BTNL3 | CD79b |
| OX40 | NKG2D | CD8 |
| OX40 | NKG2D | CD3ζ |
| OX40 | NKG2D | CD3δ |
| OX40 | NKG2D | CD3γ |
| OX40 | NKG2D | CD3ε |
| OX40 | NKG2D | FcγRI-γ |
| OX40 | NKG2D | FcγRIII-γ |
| OX40 | NKG2D | FcεRIβ |
| OX40 | NKG2D | FcεRIγ |
| OX40 | NKG2D | DAP10 |
| OX40 | NKG2D | DAP12 |
| OX40 | NKG2D | CD32 |
| OX40 | NKG2D | CD79a |
| OX40 | NKG2D | CD79b |
| DAP10 | CD28 | CD8 |
| DAP10 | CD28 | CD3ζ |
| DAP10 | CD28 | CD3δ |
| DAP10 | CD28 | CD3γ |
| DAP10 | CD28 | CD3ε |
| DAP10 | CD28 | FcγRI-γ |
| DAP10 | CD28 | FcγRIII-γ |
| DAP10 | CD28 | FcεRIβ |
| DAP10 | CD28 | FcεRIγ |
| DAP10 | CD28 | DAP10 |
| DAP10 | CD28 | DAP12 |
| DAP10 | CD28 | CD32 |
| DAP10 | CD28 | CD79a |
| DAP10 | CD28 | CD79b |
| DAP10 | CD8 | CD8 |
| DAP10 | CD8 | CD3ζ |
| DAP10 | CD8 | CD3δ |
| DAP10 | CD8 | CD3γ |
| DAP10 | CD8 | CD3ε |
| DAP10 | CD8 | FcγRI-γ |
| DAP10 | CD8 | FcγRIII-γ |
| DAP10 | CD8 | FcεRIβ |
| DAP10 | CD8 | FcεRIγ |
| DAP10 | CD8 | DAP10 |
| DAP10 | CD8 | DAP12 |
| DAP10 | CD8 | CD32 |
| DAP10 | CD8 | CD79a |
| DAP10 | CD8 | CD79b |
| DAP10 | CD4 | CD8 |
| DAP10 | CD4 | CD3ζ |
| DAP10 | CD4 | CD3δ |
| DAP10 | CD4 | CD3γ |
| DAP10 | CD4 | CD3ε |
| DAP10 | CD4 | FcγRI-γ |
| DAP10 | CD4 | FcγRIII-γ |
| DAP10 | CD4 | FcεRIβ |
| DAP10 | CD4 | FcεRIγ |
| DAP10 | CD4 | DAP10 |
| DAP10 | CD4 | DAP12 |
| DAP10 | CD4 | CD32 |
| DAP10 | CD4 | CD79a |
| DAP10 | CD4 | CD79b |
| DAP10 | b2c | CD8 |
| DAP10 | b2c | CD3ζ |
| DAP10 | b2c | CD3δ |
| DAP10 | b2c | CD3γ |
| DAP10 | b2c | CD3ε |
| DAP10 | b2c | FcγRI-γ |
| DAP10 | b2c | FcγRIII-γ |
| DAP10 | b2c | FcεRIβ |
| DAP10 | b2c | FcεRIγ |
| DAP10 | b2c | DAP10 |
| DAP10 | b2c | DAP12 |
| DAP10 | b2c | CD32 |
| DAP10 | b2c | CD79a |
| DAP10 | b2c | CD79b |
| DAP10 | CD137/41BB | CD8 |
| DAP10 | CD137/41BB | CD3ζ |
| DAP10 | CD137/41BB | CD3δ |
| DAP10 | CD137/41BB | CD3γ |
| DAP10 | CD137/41BB | CD3ε |
| DAP10 | CD137/41BB | FcγRI-γ |
| DAP10 | CD137/41BB | FcγRIII-γ |
| DAP10 | CD137/41BB | FcεRIβ |
| DAP10 | CD137/41BB | FcεRIγ |
| DAP10 | CD137/41BB | DAP10 |
| DAP10 | CD137/41BB | DAP12 |
| DAP10 | CD137/41BB | CD32 |
| DAP10 | CD137/41BB | CD79a |
| DAP10 | CD137/41BB | CD79b |
| DAP10 | ICOS | CD8 |
| DAP10 | ICOS | CD3ζ |
| DAP10 | ICOS | CD3δ |
| DAP10 | ICOS | CD3γ |
| DAP10 | ICOS | CD3ε |
| DAP10 | ICOS | FcγRI-γ |
| DAP10 | ICOS | FcγRIII-γ |
| DAP10 | ICOS | FcεRIβ |
| DAP10 | ICOS | FcεRIγ |
| DAP10 | ICOS | DAP10 |
| DAP10 | ICOS | DAP12 |
| DAP10 | ICOS | CD32 |
| DAP10 | ICOS | CD79a |
| DAP10 | ICOS | CD79b |
| DAP10 | CD27 | CD8 |
| DAP10 | CD27 | CD3ζ |
| DAP10 | CD27 | CD3δ |
| DAP10 | CD27 | CD3γ |
| DAP10 | CD27 | CD3ε |
| DAP10 | CD27 | FcγRI-γ |
| DAP10 | CD27 | FcγRIII-γ |
| DAP10 | CD27 | FcεRIβ |
| DAP10 | CD27 | FcεRIγ |
| DAP10 | CD27 | DAP10 |
| DAP10 | CD27 | DAP12 |
| DAP10 | CD27 | CD32 |
| DAP10 | CD27 | CD79a |
| DAP10 | CD27 | CD79b |
| DAP10 | CD28δ | CD8 |
| DAP10 | CD28δ | CD3ζ |
| DAP10 | CD28δ | CD3δ |
| DAP10 | CD28δ | CD3γ |
| DAP10 | CD28δ | CD3ε |
| DAP10 | CD28δ | FcγRI-γ |
| DAP10 | CD28δ | FcγRIII-γ |
| DAP10 | CD28δ | FcεRIβ |
| DAP10 | CD28δ | FcεRIγ |
| DAP10 | CD28δ | DAP10 |
| DAP10 | CD28δ | DAP12 |
| DAP10 | CD28δ | CD32 |
| DAP10 | CD28δ | CD79a |
| DAP10 | CD28δ | CD79b |
| DAP10 | CD80 | CD8 |
| DAP10 | CD80 | CD3ζ |
| DAP10 | CD80 | CD3δ |
| DAP10 | CD80 | CD3γ |
| DAP10 | CD80 | CD3ε |
| DAP10 | CD80 | FcγRI-γ |
| DAP10 | CD80 | FcγRIII-γ |
| DAP10 | CD80 | FcεRIβ |
| DAP10 | CD80 | FcεRIγ |
| DAP10 | CD80 | DAP10 |
| DAP10 | CD80 | DAP12 |
| DAP10 | CD80 | CD32 |
| DAP10 | CD80 | CD79a |
| DAP10 | CD80 | CD79b |
| DAP10 | CD86 | CD8 |
| DAP10 | CD86 | CD3ζ |
| DAP10 | CD86 | CD3δ |
| DAP10 | CD86 | CD3γ |
| DAP10 | CD86 | CD3ε |
| DAP10 | CD86 | FcγRI-γ |
| DAP10 | CD86 | FcγRIII-γ |
| DAP10 | CD86 | FcεRIβ |
| DAP10 | CD86 | FcεRIγ |
| DAP10 | CD86 | DAP10 |
| DAP10 | CD86 | DAP12 |
| DAP10 | CD86 | CD32 |
| DAP10 | CD86 | CD79a |
| DAP10 | CD86 | CD79b |
| DAP10 | OX40 | CD8 |
| DAP10 | OX40 | CD3ζ |
| DAP10 | OX40 | CD3δ |
| DAP10 | OX40 | CD3γ |
| DAP10 | OX40 | CD3ε |
| DAP10 | OX40 | FcγRI-γ |
| DAP10 | OX40 | FcγRIII-γ |
| DAP10 | OX40 | FcεRIβ |
| DAP10 | OX40 | FcεRIγ |
| DAP10 | OX40 | DAP10 |
| DAP10 | OX40 | DAP12 |
| DAP10 | OX40 | CD32 |
| DAP10 | OX40 | CD79a |
| DAP10 | OX40 | CD79b |
| DAP10 | DAP10 | CD8 |
| DAP10 | DAP10 | CD3ζ |
| DAP10 | DAP10 | CD3δ |
| DAP10 | DAP10 | CD3γ |
| DAP10 | DAP10 | CD3ε |
| DAP10 | DAP10 | FcγRI-γ |
| DAP10 | DAP10 | FcγRIII-γ |
| DAP10 | DAP10 | FcεRIβ |
| DAP10 | DAP10 | FcεRIγ |
| DAP10 | DAP10 | DAP10 |
| DAP10 | DAP10 | DAP12 |
| DAP10 | DAP10 | CD32 |
| DAP10 | DAP10 | CD79a |
| DAP10 | DAP10 | CD79b |
| DAP10 | DAP12 | CD8 |
| DAP10 | DAP12 | CD3ζ |
| DAP10 | DAP12 | CD3δ |
| DAP10 | DAP12 | CD3γ |
| DAP10 | DAP12 | CD3ε |
| DAP10 | DAP12 | FcγRI-γ |
| DAP10 | DAP12 | FcγRIII-γ |
| DAP10 | DAP12 | FcεRIβ |
| DAP10 | DAP12 | FcεRIγ |
| DAP10 | DAP12 | DAP10 |
| DAP10 | DAP12 | DAP12 |
| DAP10 | DAP12 | CD32 |
| DAP10 | DAP12 | CD79a |
| DAP10 | DAP12 | CD79b |
| DAP10 | MyD88 | CD8 |
| DAP10 | MyD88 | CD3ζ |
| DAP10 | MyD88 | CD3δ |
| DAP10 | MyD88 | CD3γ |
| DAP10 | MyD88 | CD3ε |
| DAP10 | MyD88 | FcγRI-γ |
| DAP10 | MyD88 | FcγRIII-γ |
| DAP10 | MyD88 | FcεRIβ |
| DAP10 | MyD88 | FcεRIγ |
| DAP10 | MyD88 | DAP10 |
| DAP10 | MyD88 | DAP12 |
| DAP10 | MyD88 | CD32 |
| DAP10 | MyD88 | CD79a |
| DAP10 | MyD88 | CD79b |
| DAP10 | CD7 | CD8 |
| DAP10 | CD7 | CD3ζ |
| DAP10 | CD7 | CD3δ |
| DAP10 | CD7 | CD3γ |
| DAP10 | CD7 | CD3ε |
| DAP10 | CD7 | FcγRI-γ |
| DAP10 | CD7 | FcγRIII-γ |
| DAP10 | CD7 | FcεRIβ |
| DAP10 | CD7 | FcεRIγ |
| DAP10 | CD7 | DAP10 |
| DAP10 | CD7 | DAP12 |
| DAP10 | CD7 | CD32 |
| DAP10 | CD7 | CD79a |
| DAP10 | CD7 | CD79b |
| DAP10 | BTNL3 | CD8 |
| DAP10 | BTNL3 | CD3ζ |
| DAP10 | BTNL3 | CD3δ |
| DAP10 | BTNL3 | CD3γ |
| DAP10 | BTNL3 | CD3ε |
| DAP10 | BTNL3 | FcγRI-γ |
| DAP10 | BTNL3 | FcγRIII-γ |
| DAP10 | BTNL3 | FcεRIβ |
| DAP10 | BTNL3 | FcεRIγ |
| DAP10 | BTNL3 | DAP10 |
| DAP10 | BTNL3 | DAP12 |
| DAP10 | BTNL3 | CD32 |
| DAP10 | BTNL3 | CD79a |
| DAP10 | BTNL3 | CD79b |
| DAP10 | NKG2D | CD8 |
| DAP10 | NKG2D | CD3ζ |
| DAP10 | NKG2D | CD3δ |
| DAP10 | NKG2D | CD3γ |
| DAP10 | NKG2D | CD3ε |
| DAP10 | NKG2D | FcγRI-γ |
| DAP10 | NKG2D | FcγRIII-γ |
| DAP10 | NKG2D | FcεRIβ |
| DAP10 | NKG2D | FcεRIγ |
| DAP10 | NKG2D | DAP10 |
| DAP10 | NKG2D | DAP12 |
| DAP10 | NKG2D | CD32 |
| DAP10 | NKG2D | CD79a |
| DAP10 | NKG2D | CD79b |
| DAP12 | CD28 | CD8 |
| DAP12 | CD28 | CD3ζ |
| DAP12 | CD28 | CD3δ |
| DAP12 | CD28 | CD3γ |
| DAP12 | CD28 | CD3ε |
| DAP12 | CD28 | FcγRI-γ |
| DAP12 | CD28 | FcγRIII-γ |
| DAP12 | CD28 | FcεRIβ |
| DAP12 | CD28 | FcεRIγ |
| DAP12 | CD28 | DAP10 |
| DAP12 | CD28 | DAP12 |
| DAP12 | CD28 | CD32 |
| DAP12 | CD28 | CD79a |
| DAP12 | CD28 | CD79b |
| DAP12 | CD8 | CD8 |
| DAP12 | CD8 | CD3ζ |
| DAP12 | CD8 | CD3δ |
| DAP12 | CD8 | CD3γ |
| DAP12 | CD8 | CD3ε |
| DAP12 | CD8 | FcγRI-γ |
| DAP12 | CD8 | FcγRIII-γ |
| DAP12 | CD8 | FcεRIβ |
| DAP12 | CD8 | FcεRIγ |
| DAP12 | CD8 | DAP10 |
| DAP12 | CD8 | DAP12 |
| DAP12 | CD8 | CD32 |
| DAP12 | CD8 | CD79a |
| DAP12 | CD8 | CD79b |
| DAP12 | CD4 | CD8 |
| DAP12 | CD4 | CD3ζ |
| DAP12 | CD4 | CD3δ |
| DAP12 | CD4 | CD3γ |
| DAP12 | CD4 | CD3ε |
| DAP12 | CD4 | FcγRI-γ |
| DAP12 | CD4 | FcγRIII-γ |
| DAP12 | CD4 | FcεRIβ |
| DAP12 | CD4 | FcεRIγ |
| DAP12 | CD4 | DAP10 |
| DAP12 | CD4 | DAP12 |
| DAP12 | CD4 | CD32 |
| DAP12 | CD4 | CD79a |
| DAP12 | CD4 | CD79b |
| DAP12 | b2c | CD8 |
| DAP12 | b2c | CD3ζ |
| DAP12 | b2c | CD3δ |
| DAP12 | b2c | CD3γ |
| DAP12 | b2c | CD3ε |
| DAP12 | b2c | FcγRI-γ |
| DAP12 | b2c | FcγRIII-γ |
| DAP12 | b2c | FcεRIβ |
| DAP12 | b2c | FcεRIγ |
| DAP12 | b2c | DAP10 |
| DAP12 | b2c | DAP12 |
| DAP12 | b2c | CD32 |
| DAP12 | b2c | CD79a |
| DAP12 | b2c | CD79b |
| DAP12 | CD137/41BB | CD8 |
| DAP12 | CD137/41BB | CD3ζ |
| DAP12 | CD137/41BB | CD3δ |
| DAP12 | CD137/41BB | CD3γ |
| DAP12 | CD137/41BB | CD3ε |
| DAP12 | CD137/41BB | FcγRI-γ |
| DAP12 | CD137/41BB | FcγRIII-γ |
| DAP12 | CD137/41BB | FcεRIβ |
| DAP12 | CD137/41BB | FcεRIγ |
| DAP12 | CD137/41BB | DAP10 |
| DAP12 | CD137/41BB | DAP12 |
| DAP12 | CD137/41BB | CD32 |
| DAP12 | CD137/41BB | CD79a |
| DAP12 | CD137/41BB | CD79b |
| DAP12 | ICOS | CD8 |
| DAP12 | ICOS | CD3ζ |
| DAP12 | ICOS | CD3δ |
| DAP12 | ICOS | CD3γ |
| DAP12 | ICOS | CD3ε |
| DAP12 | ICOS | FcγRI-γ |
| DAP12 | ICOS | FcγRIII-γ |
| DAP12 | ICOS | FcεRIβ |
| DAP12 | ICOS | FcεRIγ |
| DAP12 | ICOS | DAP10 |
| DAP12 | ICOS | DAP12 |
| DAP12 | ICOS | CD32 |
| DAP12 | ICOS | CD79a |
| DAP12 | ICOS | CD79b |
| DAP12 | CD27 | CD8 |
| DAP12 | CD27 | CD3ζ |
| DAP12 | CD27 | CD3δ |
| DAP12 | CD27 | CD3γ |
| DAP12 | CD27 | CD3ε |
| DAP12 | CD27 | FcγRI-γ |
| DAP12 | CD27 | FcγRIII-γ |
| DAP12 | CD27 | FcεRIβ |
| DAP12 | CD27 | FcεRIγ |
| DAP12 | CD27 | DAP10 |
| DAP12 | CD27 | DAP12 |
| DAP12 | CD27 | CD32 |
| DAP12 | CD27 | CD79a |
| DAP12 | CD27 | CD79b |
| DAP12 | CD28δ | CD8 |
| DAP12 | CD28δ | CD3ζ |
| DAP12 | CD28δ | CD3δ |
| DAP12 | CD28δ | CD3γ |
| DAP12 | CD28δ | CD3ε |
| DAP12 | CD28δ | FcγRI-γ |
| DAP12 | CD28δ | FcγRIII-γ |
| DAP12 | CD28δ | FcεRIβ |
| DAP12 | CD28δ | FcεRIγ |
| DAP12 | CD28δ | DAP10 |
| DAP12 | CD28δ | DAP12 |
| DAP12 | CD28δ | CD32 |
| DAP12 | CD28δ | CD79a |
| DAP12 | CD28δ | CD79b |
| DAP12 | CD80 | CD8 |
| DAP12 | CD80 | CD3ζ |
| DAP12 | CD80 | CD3δ |
| DAP12 | CD80 | CD3γ |
| DAP12 | CD80 | CD3ε |
| DAP12 | CD80 | FcγRI-γ |
| DAP12 | CD80 | FcγRIII-γ |
| DAP12 | CD80 | FcεRIβ |
| DAP12 | CD80 | FcεRIγ |
| DAP12 | CD80 | DAP10 |
| DAP12 | CD80 | DAP12 |
| DAP12 | CD80 | CD32 |
| DAP12 | CD80 | CD79a |
| DAP12 | CD80 | CD79b |
| DAP12 | CD86 | CD8 |
| DAP12 | CD86 | CD3ζ |
| DAP12 | CD86 | CD3δ |
| DAP12 | CD86 | CD3γ |
| DAP12 | CD86 | CD3ε |
| DAP12 | CD86 | FcγRI-γ |
| DAP12 | CD86 | FcγRIII-γ |
| DAP12 | CD86 | FcεRIβ |
| DAP12 | CD86 | FcεRIγ |
| DAP12 | CD86 | DAP10 |
| DAP12 | CD86 | DAP12 |
| DAP12 | CD86 | CD32 |
| DAP12 | CD86 | CD79a |
| DAP12 | CD86 | CD79b |
| DAP12 | OX40 | CD8 |
| DAP12 | OX40 | CD3ζ |
| DAP12 | OX40 | CD3δ |
| DAP12 | OX40 | CD3γ |
| DAP12 | OX40 | CD3ε |
| DAP12 | OX40 | FcγRI-γ |
| DAP12 | OX40 | FcγRIII-γ |
| DAP12 | OX40 | FcεRIβ |
| DAP12 | OX40 | FcεRIγ |
| DAP12 | OX40 | DAP10 |
| DAP12 | OX40 | DAP12 |
| DAP12 | OX40 | CD32 |
| DAP12 | OX40 | CD79a |
| DAP12 | OX40 | CD79b |
| DAP12 | DAP10 | CD8 |
| DAP12 | DAP10 | CD3ζ |
| DAP12 | DAP10 | CD3δ |
| DAP12 | DAP10 | CD3γ |
| DAP12 | DAP10 | CD3ε |
| DAP12 | DAP10 | FcγRI-γ |
| DAP12 | DAP10 | FcγRIII-γ |
| DAP12 | DAP10 | FcεRIβ |
| DAP12 | DAP10 | FcεRIγ |
| DAP12 | DAP10 | DAP10 |
| DAP12 | DAP10 | DAP12 |
| DAP12 | DAP10 | CD32 |
| DAP12 | DAP10 | CD79a |
| DAP12 | DAP10 | CD79b |
| DAP12 | DAP12 | CD8 |
| DAP12 | DAP12 | CD3ζ |
| DAP12 | DAP12 | CD3δ |
| DAP12 | DAP12 | CD3γ |
| DAP12 | DAP12 | CD3ε |
| DAP12 | DAP12 | FcγRI-γ |
| DAP12 | DAP12 | FcγRIII-γ |
| DAP12 | DAP12 | FcεRIβ |
| DAP12 | DAP12 | FcεRIγ |
| DAP12 | DAP12 | DAP10 |
| DAP12 | DAP12 | DAP12 |
| DAP12 | DAP12 | CD32 |
| DAP12 | DAP12 | CD79a |
| DAP12 | DAP12 | CD79b |
| DAP12 | MyD88 | CD8 |
| DAP12 | MyD88 | CD3ζ |
| DAP12 | MyD88 | CD3δ |
| DAP12 | MyD88 | CD3γ |
| DAP12 | MyD88 | CD3ε |
| DAP12 | MyD88 | FcγRI-γ |
| DAP12 | MyD88 | FcγRIII-γ |
| DAP12 | MyD88 | FcεRIβ |
| DAP12 | MyD88 | FcεRIγ |
| DAP12 | MyD88 | DAP10 |
| DAP12 | MyD88 | DAP12 |
| DAP12 | MyD88 | CD32 |
| DAP12 | MyD88 | CD79a |
| DAP12 | MyD88 | CD79b |
| DAP12 | CD7 | CD8 |
| DAP12 | CD7 | CD3ζ |
| DAP12 | CD7 | CD3δ |
| DAP12 | CD7 | CD3γ |
| DAP12 | CD7 | CD3ε |
| DAP12 | CD7 | FcγRI-γ |
| DAP12 | CD7 | FcγRIII-γ |
| DAP12 | CD7 | FcεRIβ |
| DAP12 | CD7 | FcεRIγ |
| DAP12 | CD7 | DAP10 |
| DAP12 | CD7 | DAP12 |
| DAP12 | CD7 | CD32 |
| DAP12 | CD7 | CD79a |
| DAP12 | CD7 | CD79b |
| DAP12 | BTNL3 | CD8 |
| DAP12 | BTNL3 | CD3ζ |
| DAP12 | BTNL3 | CD3δ |
| DAP12 | BTNL3 | CD3γ |
| DAP12 | BTNL3 | CD3ε |
| DAP12 | BTNL3 | FcγRI-γ |
| DAP12 | BTNL3 | FcγRIII-γ |
| DAP12 | BTNL3 | FcεRIβ |
| DAP12 | BTNL3 | FcεRIγ |
| DAP12 | BTNL3 | DAP10 |
| DAP12 | BTNL3 | DAP12 |
| DAP12 | BTNL3 | CD32 |
| DAP12 | BTNL3 | CD79a |
| DAP12 | BTNL3 | CD79b |
| DAP12 | NKG2D | CD8 |
| DAP12 | NKG2D | CD3ζ |
| DAP12 | NKG2D | CD3δ |
| DAP12 | NKG2D | CD3γ |
| DAP12 | NKG2D | CD3ε |
| DAP12 | NKG2D | FcγRI-γ |
| DAP12 | NKG2D | FcγRIII-γ |
| DAP12 | NKG2D | FcεRIβ |
| DAP12 | NKG2D | FcεRIγ |
| DAP12 | NKG2D | DAP10 |
| DAP12 | NKG2D | DAP12 |
| DAP12 | NKG2D | CD32 |
| DAP12 | NKG2D | CD79a |
| DAP12 | NKG2D | CD79b |
| MyD88 | CD28 | CD8 |
| MyD88 | CD28 | CD3ζ |
| MyD88 | CD28 | CD3δ |
| MyD88 | CD28 | CD3γ |
| MyD88 | CD28 | CD3ε |
| MyD88 | CD28 | FcγRI-γ |
| MyD88 | CD28 | FcγRIII-γ |
| MyD88 | CD28 | FcεRIβ |
| MyD88 | CD28 | FcεRIγ |
| MyD88 | CD28 | DAP10 |
| MyD88 | CD28 | DAP12 |
| MyD88 | CD28 | CD32 |
| MyD88 | CD28 | CD79a |
| MyD88 | CD28 | CD79b |
| MyD88 | CD8 | CD8 |
| MyD88 | CD8 | CD3ζ |
| MyD88 | CD8 | CD3δ |
| MyD88 | CD8 | CD3γ |
| MyD88 | CD8 | CD3ε |
| MyD88 | CD8 | FcγRI-γ |
| MyD88 | CD8 | FcγRIII-γ |
| MyD88 | CD8 | FcεRIβ |
| MyD88 | CD8 | FcεRIγ |
| MyD88 | CD8 | DAP10 |
| MyD88 | CD8 | DAP12 |
| MyD88 | CD8 | CD32 |
| MyD88 | CD8 | CD79a |
| MyD88 | CD8 | CD79b |
| MyD88 | CD4 | CD8 |
| MyD88 | CD4 | CD3ζ |
| MyD88 | CD4 | CD3δ |
| MyD88 | CD4 | CD3γ |
| MyD88 | CD4 | CD3ε |
| MyD88 | CD4 | FcγRI-γ |
| MyD88 | CD4 | FcγRIII-γ |
| MyD88 | CD4 | FcεRIβ |
| MyD88 | CD4 | FcεRIγ |
| MyD88 | CD4 | DAP10 |
| MyD88 | CD4 | DAP12 |
| MyD88 | CD4 | CD32 |
| MyD88 | CD4 | CD79a |
| MyD88 | CD4 | CD79b |
| MyD88 | b2c | CD8 |
| MyD88 | b2c | CD3ζ |
| MyD88 | b2c | CD3δ |
| MyD88 | b2c | CD3γ |
| MyD88 | b2c | CD3ε |
| MyD88 | b2c | FcγRI-γ |
| MyD88 | b2c | FcγRIII-γ |
| MyD88 | b2c | FcεRIβ |
| MyD88 | b2c | FcεRIγ |
| MyD88 | b2c | DAP10 |
| MyD88 | b2c | DAP12 |
| MyD88 | b2c | CD32 |
| MyD88 | b2c | CD79a |
| MyD88 | b2c | CD79b |
| MyD88 | CD137/41BB | CD8 |
| MyD88 | CD137/41BB | CD3ζ |
| MyD88 | CD137/41BB | CD3δ |
| MyD88 | CD137/41BB | CD3γ |
| MyD88 | CD137/41BB | CD3ε |
| MyD88 | CD137/41BB | FcγRI-γ |
| MyD88 | CD137/41BB | FcγRIII-γ |
| MyD88 | CD137/41BB | FcεRIβ |
| MyD88 | CD137/41BB | FcεRIγ |
| MyD88 | CD137/41BB | DAP10 |
| MyD88 | CD137/41BB | DAP12 |
| MyD88 | CD137/41BB | CD32 |
| MyD88 | CD137/41BB | CD79a |
| MyD88 | CD137/41BB | CD79b |
| MyD88 | ICOS | CD8 |
| MyD88 | ICOS | CD3ζ |
| MyD88 | ICOS | CD3δ |
| MyD88 | ICOS | CD3γ |
| MyD88 | ICOS | CD3ε |
| MyD88 | ICOS | FcγRI-γ |
| MyD88 | ICOS | FcγRIII-γ |
| MyD88 | ICOS | FcεRIβ |
| MyD88 | ICOS | FcεRIγ |
| MyD88 | ICOS | DAP10 |
| MyD88 | ICOS | DAP12 |
| MyD88 | ICOS | CD32 |
| MyD88 | ICOS | CD79a |
| MyD88 | ICOS | CD79b |
| MyD88 | CD27 | CD8 |
| MyD88 | CD27 | CD3ζ |
| MyD88 | CD27 | CD3δ |
| MyD88 | CD27 | CD3γ |
| MyD88 | CD27 | CD3ε |
| MyD88 | CD27 | FcγRI-γ |
| MyD88 | CD27 | FcγRIII-γ |
| MyD88 | CD27 | FcεRIβ |
| MyD88 | CD27 | FcεRIγ |
| MyD88 | CD27 | DAP10 |
| MyD88 | CD27 | DAP12 |
| MyD88 | CD27 | CD32 |
| MyD88 | CD27 | CD79a |
| MyD88 | CD27 | CD79b |
| MyD88 | CD28δ | CD8 |
| MyD88 | CD28δ | CD3ζ |
| MyD88 | CD28δ | CD3δ |
| MyD88 | CD28δ | CD3γ |
| MyD88 | CD28δ | CD3ε |
| MyD88 | CD28δ | FcγRI-γ |
| MyD88 | CD28δ | FcγRIII-γ |
| MyD88 | CD28δ | FcεRIβ |
| MyD88 | CD28δ | FcεRIγ |
| MyD88 | CD28δ | DAP10 |
| MyD88 | CD28δ | DAP12 |
| MyD88 | CD28δ | CD32 |
| MyD88 | CD28δ | CD79a |
| MyD88 | CD28δ | CD79b |
| MyD88 | CD80 | CD8 |
| MyD88 | CD80 | CD3ζ |
| MyD88 | CD80 | CD3δ |
| MyD88 | CD80 | CD3γ |
| MyD88 | CD80 | CD3ε |
| MyD88 | CD80 | FcγRI-γ |
| MyD88 | CD80 | FcγRIII-γ |
| MyD88 | CD80 | FcεRIβ |
| MyD88 | CD80 | FcεRIγ |
| MyD88 | CD80 | DAP10 |
| MyD88 | CD80 | DAP12 |
| MyD88 | CD80 | CD32 |
| MyD88 | CD80 | CD79a |
| MyD88 | CD80 | CD79b |
| MyD88 | CD86 | CD8 |
| MyD88 | CD86 | CD3ζ |
| MyD88 | CD86 | CD3δ |
| MyD88 | CD86 | CD3γ |
| MyD88 | CD86 | CD3ε |
| MyD88 | CD86 | FcγRI-γ |
| MyD88 | CD86 | FcγRIII-γ |
| MyD88 | CD86 | FcεRIβ |
| MyD88 | CD86 | FcεRIγ |
| MyD88 | CD86 | DAP10 |
| MyD88 | CD86 | DAP12 |
| MyD88 | CD86 | CD32 |
| MyD88 | CD86 | CD79a |
| MyD88 | CD86 | CD79b |
| MyD88 | OX40 | CD8 |
| MyD88 | OX40 | CD3ζ |
| MyD88 | OX40 | CD3δ |
| MyD88 | OX40 | CD3γ |
| MyD88 | OX40 | CD3ε |
| MyD88 | OX40 | FcγRI-γ |
| MyD88 | OX40 | FcγRIII-γ |
| MyD88 | OX40 | FcεRIβ |
| MyD88 | OX40 | FcεRIγ |
| MyD88 | OX40 | DAP10 |
| MyD88 | OX40 | DAP12 |
| MyD88 | OX40 | CD32 |
| MyD88 | OX40 | CD79a |
| MyD88 | OX40 | CD79b |
| MyD88 | DAP10 | CD8 |
| MyD88 | DAP10 | CD3ζ |
| MyD88 | DAP10 | CD3δ |
| MyD88 | DAP10 | CD3γ |
| MyD88 | DAP10 | CD3ε |
| MyD88 | DAP10 | FcγRI-γ |
| MyD88 | DAP10 | FcγRIII-γ |
| MyD88 | DAP10 | FcεRIβ |
| MyD88 | DAP10 | FcεRIγ |
| MyD88 | DAP10 | DAP10 |
| MyD88 | DAP10 | DAP12 |
| MyD88 | DAP10 | CD32 |
| MyD88 | DAP10 | CD79a |
| MyD88 | DAP10 | CD79b |
| MyD88 | DAP12 | CD8 |
| MyD88 | DAP12 | CD3ζ |
| MyD88 | DAP12 | CD3δ |
| MyD88 | DAP12 | CD3γ |
| MyD88 | DAP12 | CD3ε |
| MyD88 | DAP12 | FcγRI-γ |
| MyD88 | DAP12 | FcγRIII-γ |
| MyD88 | DAP12 | FcεRIβ |
| MyD88 | DAP12 | FcεRIγ |
| MyD88 | DAP12 | DAP10 |
| MyD88 | DAP12 | DAP12 |
| MyD88 | DAP12 | CD32 |
| MyD88 | DAP12 | CD79a |
| MyD88 | DAP12 | CD79b |
| MyD88 | MyD88 | CD8 |
| MyD88 | MyD88 | CD3ζ |
| MyD88 | MyD88 | CD3δ |
| MyD88 | MyD88 | CD3γ |
| MyD88 | MyD88 | CD3ε |
| MyD88 | MyD88 | FcγRI-γ |
| MyD88 | MyD88 | FcγRIII-γ |
| MyD88 | MyD88 | FcεRIβ |
| MyD88 | MyD88 | FcεRIγ |
| MyD88 | MyD88 | DAP10 |
| MyD88 | MyD88 | DAP12 |
| MyD88 | MyD88 | CD32 |
| MyD88 | MyD88 | CD79a |
| MyD88 | MyD88 | CD79b |
| MyD88 | CD7 | CD8 |
| MyD88 | CD7 | CD3ζ |
| MyD88 | CD7 | CD3δ |
| MyD88 | CD7 | CD3γ |
| MyD88 | CD7 | CD3ε |
| MyD88 | CD7 | FcγRI-γ |
| MyD88 | CD7 | FcγRIII-γ |
| MyD88 | CD7 | FcεRIβ |
| MyD88 | CD7 | FcεRIγ |
| MyD88 | CD7 | DAP10 |
| MyD88 | CD7 | DAP12 |
| MyD88 | CD7 | CD32 |
| MyD88 | CD7 | CD79a |
| MyD88 | CD7 | CD79b |
| MyD88 | BTNL3 | CD8 |
| MyD88 | BTNL3 | CD3ζ |
| MyD88 | BTNL3 | CD3δ |
| MyD88 | BTNL3 | CD3γ |
| MyD88 | BTNL3 | CD3ε |
| MyD88 | BTNL3 | FcγRI-γ |
| MyD88 | BTNL3 | FcγRIII-γ |
| MyD88 | BTNL3 | FcεRIβ |
| MyD88 | BTNL3 | FcεRIγ |
| MyD88 | BTNL3 | DAP10 |
| MyD88 | BTNL3 | DAP12 |
| MyD88 | BTNL3 | CD32 |
| MyD88 | BTNL3 | CD79a |
| MyD88 | BTNL3 | CD79b |
| MyD88 | NKG2D | CD8 |
| MyD88 | NKG2D | CD3ζ |
| MyD88 | NKG2D | CD3δ |
| MyD88 | NKG2D | CD3γ |
| MyD88 | NKG2D | CD3ε |
| MyD88 | NKG2D | FcγRI-γ |
| MyD88 | NKG2D | FcγRIII-γ |
| MyD88 | NKG2D | FcεRIβ |
| MyD88 | NKG2D | FcεRIγ |
| MyD88 | NKG2D | DAP10 |
| MyD88 | NKG2D | DAP12 |
| MyD88 | NKG2D | CD32 |
| MyD88 | NKG2D | CD79a |
| MyD88 | NKG2D | CD79b |
| CD7 | CD28 | CD8 |
| CD7 | CD28 | CD3ζ |
| CD7 | CD28 | CD3δ |
| CD7 | CD28 | CD3γ |
| CD7 | CD28 | CD3ε |
| CD7 | CD28 | FcγRI-γ |
| CD7 | CD28 | FcγRIII-γ |
| CD7 | CD28 | FcεRIβ |
| CD7 | CD28 | FcεRIγ |
| CD7 | CD28 | DAP10 |
| CD7 | CD28 | DAP12 |
| CD7 | CD28 | CD32 |
| CD7 | CD28 | CD79a |
| CD7 | CD28 | CD79b |
| CD7 | CD8 | CD8 |
| CD7 | CD8 | CD3ζ |
| CD7 | CD8 | CD3δ |
| CD7 | CD8 | CD3γ |
| CD7 | CD8 | CD3ε |
| CD7 | CD8 | FcγRI-γ |
| CD7 | CD8 | FcγRIII-γ |
| CD7 | CD8 | FcεRIβ |
| CD7 | CD8 | FcεRIγ |
| CD7 | CD8 | DAP10 |
| CD7 | CD8 | DAP12 |
| CD7 | CD8 | CD32 |
| CD7 | CD8 | CD79a |
| CD7 | CD8 | CD79b |
| CD7 | CD4 | CD8 |
| CD7 | CD4 | CD3ζ |
| CD7 | CD4 | CD3δ |
| CD7 | CD4 | CD3γ |
| CD7 | CD4 | CD3ε |
| CD7 | CD4 | FcγRI-γ |
| CD7 | CD4 | FcγRIII-γ |
| CD7 | CD4 | FcεRIβ |
| CD7 | CD4 | FcεRIγ |
| CD7 | CD4 | DAP10 |
| CD7 | CD4 | DAP12 |
| CD7 | CD4 | CD32 |
| CD7 | CD4 | CD79a |
| CD7 | CD4 | CD79b |
| CD7 | b2c | CD8 |
| CD7 | b2c | CD3ζ |
| CD7 | b2c | CD3δ |
| CD7 | b2c | CD3γ |
| CD7 | b2c | CD3ε |
| CD7 | b2c | FcγRI-γ |
| CD7 | b2c | FcγRIII-γ |
| CD7 | b2c | FcεRIβ |
| CD7 | b2c | FcεRIγ |
| CD7 | b2c | DAP10 |
| CD7 | b2c | DAP12 |
| CD7 | b2c | CD32 |
| CD7 | b2c | CD79a |
| CD7 | b2c | CD79b |
| CD7 | CD137/41BB | CD8 |
| CD7 | CD137/41BB | CD3ζ |
| CD7 | CD137/41BB | CD3δ |
| CD7 | CD137/41BB | CD3γ |
| CD7 | CD137/41BB | CD3ε |
| CD7 | CD137/41BB | FcγRI-γ |
| CD7 | CD137/41BB | FcγRIII-γ |
| CD7 | CD137/41BB | FcεRIβ |
| CD7 | CD137/41BB | FcεRIγ |
| CD7 | CD137/41BB | DAP10 |
| CD7 | CD137/41BB | DAP12 |
| CD7 | CD137/41BB | CD32 |
| CD7 | CD137/41BB | CD79a |
| CD7 | CD137/41BB | CD79b |
| CD7 | ICOS | CD8 |
| CD7 | ICOS | CD3ζ |
| CD7 | ICOS | CD3δ |
| CD7 | ICOS | CD3γ |
| CD7 | ICOS | CD3ε |
| CD7 | ICOS | FcγRI-γ |
| CD7 | ICOS | FcγRIII-γ |
| CD7 | ICOS | FcεRIβ |
| CD7 | ICOS | FcεRIγ |
| CD7 | ICOS | DAP10 |
| CD7 | ICOS | DAP12 |
| CD7 | ICOS | CD32 |
| CD7 | ICOS | CD79a |
| CD7 | ICOS | CD79b |
| CD7 | CD27 | CD8 |
| CD7 | CD27 | CD3ζ |
| CD7 | CD27 | CD3δ |
| CD7 | CD27 | CD3γ |
| CD7 | CD27 | CD3ε |
| CD7 | CD27 | FcγRI-γ |
| CD7 | CD27 | FcγRIII-γ |
| CD7 | CD27 | FcεRIβ |
| CD7 | CD27 | FcεRIγ |
| CD7 | CD27 | DAP10 |
| CD7 | CD27 | DAP12 |
| CD7 | CD27 | CD32 |
| CD7 | CD27 | CD79a |
| CD7 | CD27 | CD79b |
| CD7 | CD28δ | CD8 |
| CD7 | CD28δ | CD3ζ |
| CD7 | CD28δ | CD3δ |
| CD7 | CD28δ | CD3γ |
| CD7 | CD28δ | CD3ε |
| CD7 | CD28δ | FcγRI-γ |
| CD7 | CD28δ | FcγRIII-γ |
| CD7 | CD28δ | FcεRIβ |
| CD7 | CD28δ | FcεRIγ |
| CD7 | CD28δ | DAP10 |
| CD7 | CD28δ | DAP12 |
| CD7 | CD28δ | CD32 |
| CD7 | CD28δ | CD79a |
| CD7 | CD28δ | CD79b |
| CD7 | CD80 | CD8 |
| CD7 | CD80 | CD3ζ |
| CD7 | CD80 | CD3δ |
| CD7 | CD80 | CD3γ |
| CD7 | CD80 | CD3ε |
| CD7 | CD80 | FcγRI-γ |
| CD7 | CD80 | FcγRIII-γ |
| CD7 | CD80 | FcεRIβ |
| CD7 | CD80 | FcεRIγ |
| CD7 | CD80 | DAP10 |
| CD7 | CD80 | DAP12 |
| CD7 | CD80 | CD32 |
| CD7 | CD80 | CD79a |
| CD7 | CD80 | CD79b |
| CD7 | CD86 | CD8 |
| CD7 | CD86 | CD3ζ |
| CD7 | CD86 | CD3δ |
| CD7 | CD86 | CD3γ |
| CD7 | CD86 | CD3ε |
| CD7 | CD86 | FcγRI-γ |
| CD7 | CD86 | FcγRIII-γ |
| CD7 | CD86 | FcεRIβ |
| CD7 | CD86 | FcεRIγ |
| CD7 | CD86 | DAP10 |
| CD7 | CD86 | DAP12 |
| CD7 | CD86 | CD32 |
| CD7 | CD86 | CD79a |
| CD7 | CD86 | CD79b |
| CD7 | OX40 | CD8 |
| CD7 | OX40 | CD3ζ |
| CD7 | OX40 | CD3δ |
| CD7 | OX40 | CD3γ |
| CD7 | OX40 | CD3ε |
| CD7 | OX40 | FcγRI-γ |
| CD7 | OX40 | FcγRIII-γ |
| CD7 | OX40 | FcεRIβ |
| CD7 | OX40 | FcεRIγ |
| CD7 | OX40 | DAP10 |
| CD7 | OX40 | DAP12 |
| CD7 | OX40 | CD32 |
| CD7 | OX40 | CD79a |
| CD7 | OX40 | CD79b |
| CD7 | DAP10 | CD8 |
| CD7 | DAP10 | CD3ζ |
| CD7 | DAP10 | CD3δ |
| CD7 | DAP10 | CD3γ |
| CD7 | DAP10 | CD3ε |
| CD7 | DAP10 | FcγRI-γ |
| CD7 | DAP10 | FcγRIII-γ |
| CD7 | DAP10 | FcεRIβ |
| CD7 | DAP10 | FcεRIγ |
| CD7 | DAP10 | DAP10 |
| CD7 | DAP10 | DAP12 |
| CD7 | DAP10 | CD32 |
| CD7 | DAP10 | CD79a |
| CD7 | DAP10 | CD79b |
| CD7 | DAP12 | CD8 |
| CD7 | DAP12 | CD3ζ |
| CD7 | DAP12 | CD3δ |
| CD7 | DAP12 | CD3γ |
| CD7 | DAP12 | CD3ε |
| CD7 | DAP12 | FcγRI-γ |
| CD7 | DAP12 | FcγRIII-γ |
| CD7 | DAP12 | FcεRIβ |
| CD7 | DAP12 | FcεRIγ |
| CD7 | DAP12 | DAP10 |
| CD7 | DAP12 | DAP12 |
| CD7 | DAP12 | CD32 |
| CD7 | DAP12 | CD79a |
| CD7 | DAP12 | CD79b |
| CD7 | MyD88 | CD8 |
| CD7 | MyD88 | CD3ζ |
| CD7 | MyD88 | CD3δ |
| CD7 | MyD88 | CD3γ |
| CD7 | MyD88 | CD3ε |
| CD7 | MyD88 | FcγRI-γ |
| CD7 | MyD88 | FcγRIII-γ |
| CD7 | MyD88 | FcεRIβ |
| CD7 | MyD88 | FcεRIγ |
| CD7 | MyD88 | DAP10 |
| CD7 | MyD88 | DAP12 |
| CD7 | MyD88 | CD32 |
| CD7 | MyD88 | CD79a |
| CD7 | MyD88 | CD79b |
| CD7 | CD7 | CD8 |
| CD7 | CD7 | CD3ζ |
| CD7 | CD7 | CD3δ |
| CD7 | CD7 | CD3γ |
| CD7 | CD7 | CD3ε |
| CD7 | CD7 | FcγRI-γ |
| CD7 | CD7 | FcγRIII-γ |
| CD7 | CD7 | FcεRIβ |
| CD7 | CD7 | FcεRIγ |
| CD7 | CD7 | DAP10 |
| CD7 | CD7 | DAP12 |
| CD7 | CD7 | CD32 |
| CD7 | CD7 | CD79a |
| CD7 | CD7 | CD79b |
| CD7 | BTNL3 | CD8 |
| CD7 | BTNL3 | CD3ζ |
| CD7 | BTNL3 | CD3δ |
| CD7 | BTNL3 | CD3γ |
| CD7 | BTNL3 | CD3ε |
| CD7 | BTNL3 | FcγRI-γ |
| CD7 | BTNL3 | FcγRIII-γ |
| CD7 | BTNL3 | FcεRIβ |
| CD7 | BTNL3 | FcεRIγ |
| CD7 | BTNL3 | DAP10 |
| CD7 | BTNL3 | DAP12 |
| CD7 | BTNL3 | CD32 |
| CD7 | BTNL3 | CD79a |
| CD7 | BTNL3 | CD79b |
| CD7 | NKG2D | CD8 |
| CD7 | NKG2D | CD3ζ |
| CD7 | NKG2D | CD3δ |
| CD7 | NKG2D | CD3γ |
| CD7 | NKG2D | CD3ε |
| CD7 | NKG2D | FcγRI-γ |
| CD7 | NKG2D | FcγRIII-γ |
| CD7 | NKG2D | FcεRIβ |
| CD7 | NKG2D | FcεRIγ |
| CD7 | NKG2D | DAP10 |
| CD7 | NKG2D | DAP12 |
| CD7 | NKG2D | CD32 |
| CD7 | NKG2D | CD79a |
| CD7 | NKG2D | CD79b |
| BTNL3 | CD28 | CD8 |
| BTNL3 | CD28 | CD3ζ |
| BTNL3 | CD28 | CD3δ |
| BTNL3 | CD28 | CD3γ |
| BTNL3 | CD28 | CD3ε |
| BTNL3 | CD28 | FcγRI-γ |
| BTNL3 | CD28 | FcγRIII-γ |
| BTNL3 | CD28 | FcεRIβ |
| BTNL3 | CD28 | FcεRIγ |
| BTNL3 | CD28 | DAP10 |
| BTNL3 | CD28 | DAP12 |
| BTNL3 | CD28 | CD32 |
| BTNL3 | CD28 | CD79a |
| BTNL3 | CD28 | CD79b |
| BTNL3 | CD8 | CD8 |
| BTNL3 | CD8 | CD3ζ |
| BTNL3 | CD8 | CD3δ |
| BTNL3 | CD8 | CD3γ |
| BTNL3 | CD8 | CD3ε |
| BTNL3 | CD8 | FcγRI-γ |
| BTNL3 | CD8 | FcγRIII-γ |
| BTNL3 | CD8 | FcεRIβ |
| BTNL3 | CD8 | FcεRIγ |
| BTNL3 | CD8 | DAP10 |
| BTNL3 | CD8 | DAP12 |
| BTNL3 | CD8 | CD32 |
| BTNL3 | CD8 | CD79a |
| BTNL3 | CD8 | CD79b |
| BTNL3 | CD4 | CD8 |
| BTNL3 | CD4 | CD3ζ |
| BTNL3 | CD4 | CD3δ |
| BTNL3 | CD4 | CD3γ |
| BTNL3 | CD4 | CD3ε |
| BTNL3 | CD4 | FcγRI-γ |
| BTNL3 | CD4 | FcγRIII-γ |
| BTNL3 | CD4 | FcεRIβ |
| BTNL3 | CD4 | FcεRIγ |
| BTNL3 | CD4 | DAP10 |
| BTNL3 | CD4 | DAP12 |
| BTNL3 | CD4 | CD32 |
| BTNL3 | CD4 | CD79a |
| BTNL3 | CD4 | CD79b |
| BTNL3 | b2c | CD8 |
| BTNL3 | b2c | CD3ζ |
| BTNL3 | b2c | CD3δ |
| BTNL3 | b2c | CD3γ |
| BTNL3 | b2c | CD3ε |
| BTNL3 | b2c | FcγRI-γ |
| BTNL3 | b2c | FcγRIII-γ |
| BTNL3 | b2c | FcεRIβ |
| BTNL3 | b2c | FcεRIγ |
| BTNL3 | b2c | DAP10 |
| BTNL3 | b2c | DAP12 |
| BTNL3 | b2c | CD32 |
| BTNL3 | b2c | CD79a |
| BTNL3 | b2c | CD79b |
| BTNL3 | CD137/41BB | CD8 |
| BTNL3 | CD137/41BB | CD3ζ |
| BTNL3 | CD137/41BB | CD3δ |
| BTNL3 | CD137/41BB | CD3γ |
| BTNL3 | CD137/41BB | CD3ε |
| BTNL3 | CD137/41BB | FcγRI-γ |
| BTNL3 | CD137/41BB | FcγRIII-γ |
| BTNL3 | CD137/41BB | FcεRIβ |
| BTNL3 | CD137/41BB | FcεRIγ |
| BTNL3 | CD137/41BB | DAP10 |
| BTNL3 | CD137/41BB | DAP12 |
| BTNL3 | CD137/41BB | CD32 |
| BTNL3 | CD137/41BB | CD79a |
| BTNL3 | CD137/41BB | CD79b |
| BTNL3 | ICOS | CD8 |
| BTNL3 | ICOS | CD3ζ |
| BTNL3 | ICOS | CD3δ |
| BTNL3 | ICOS | CD3γ |
| BTNL3 | ICOS | CD3ε |
| BTNL3 | ICOS | FcγRI-γ |
| BTNL3 | ICOS | FcγRIII-γ |
| BTNL3 | ICOS | FcεRIβ |
| BTNL3 | ICOS | FcεRIγ |
| BTNL3 | ICOS | DAP10 |
| BTNL3 | ICOS | DAP12 |
| BTNL3 | ICOS | CD32 |
| BTNL3 | ICOS | CD79a |
| BTNL3 | ICOS | CD79b |
| BTNL3 | CD27 | CD8 |
| BTNL3 | CD27 | CD3ζ |
| BTNL3 | CD27 | CD3δ |
| BTNL3 | CD27 | CD3γ |
| BTNL3 | CD27 | CD3ε |
| BTNL3 | CD27 | FcγRI-γ |
| BTNL3 | CD27 | FcγRIII-γ |
| BTNL3 | CD27 | FcεRIβ |
| BTNL3 | CD27 | FcεRIγ |
| BTNL3 | CD27 | DAP10 |
| BTNL3 | CD27 | DAP12 |
| BTNL3 | CD27 | CD32 |
| BTNL3 | CD27 | CD79a |
| BTNL3 | CD27 | CD79b |
| BTNL3 | CD28δ | CD8 |
| BTNL3 | CD28δ | CD3ζ |
| BTNL3 | CD28δ | CD3δ |
| BTNL3 | CD28δ | CD3γ |
| BTNL3 | CD28δ | CD3ε |
| BTNL3 | CD28δ | FcγRI-γ |
| BTNL3 | CD28δ | FcγRIII-γ |
| BTNL3 | CD28δ | FcεRIβ |
| BTNL3 | CD28δ | FcεRIγ |
| BTNL3 | CD28δ | DAP10 |
| BTNL3 | CD28δ | DAP12 |
| BTNL3 | CD28δ | CD32 |
| BTNL3 | CD28δ | CD79a |
| BTNL3 | CD28δ | CD79b |
| BTNL3 | CD80 | CD8 |
| BTNL3 | CD80 | CD3ζ |
| BTNL3 | CD80 | CD3δ |
| BTNL3 | CD80 | CD3γ |
| BTNL3 | CD80 | CD3ε |
| BTNL3 | CD80 | FcγRI-γ |
| BTNL3 | CD80 | FcγRIII-γ |
| BTNL3 | CD80 | FcεRIβ |
| BTNL3 | CD80 | FcεRIγ |
| BTNL3 | CD80 | DAP10 |
| BTNL3 | CD80 | DAP12 |
| BTNL3 | CD80 | CD32 |
| BTNL3 | CD80 | CD79a |
| BTNL3 | CD80 | CD79b |
| BTNL3 | CD86 | CD8 |
| BTNL3 | CD86 | CD3ζ |
| BTNL3 | CD86 | CD3δ |
| BTNL3 | CD86 | CD3γ |
| BTNL3 | CD86 | CD3ε |
| BTNL3 | CD86 | FcγRI-γ |
| BTNL3 | CD86 | FcγRIII-γ |
| BTNL3 | CD86 | FcεRIβ |
| BTNL3 | CD86 | FcεRIγ |
| BTNL3 | CD86 | DAP10 |
| BTNL3 | CD86 | DAP12 |
| BTNL3 | CD86 | CD32 |
| BTNL3 | CD86 | CD79a |
| BTNL3 | CD86 | CD79b |
| BTNL3 | OX40 | CD8 |
| BTNL3 | OX40 | CD3ζ |
| BTNL3 | OX40 | CD3δ |
| BTNL3 | OX40 | CD3γ |
| BTNL3 | OX40 | CD3ε |
| BTNL3 | OX40 | FcγRI-γ |
| BTNL3 | OX40 | FcγRIII-γ |
| BTNL3 | OX40 | FcεRIβ |
| BTNL3 | OX40 | FcεRIγ |
| BTNL3 | OX40 | DAP10 |
| BTNL3 | OX40 | DAP12 |
| BTNL3 | OX40 | CD32 |
| BTNL3 | OX40 | CD79a |
| BTNL3 | OX40 | CD79b |
| BTNL3 | DAP10 | CD8 |
| BTNL3 | DAP10 | CD3ζ |
| BTNL3 | DAP10 | CD3δ |
| BTNL3 | DAP10 | CD3γ |
| BTNL3 | DAP10 | CD3ε |
| BTNL3 | DAP10 | FcγRI-γ |
| BTNL3 | DAP10 | FcγRIII-γ |
| BTNL3 | DAP10 | FcεRIβ |
| BTNL3 | DAP10 | FcεRIγ |
| BTNL3 | DAP10 | DAP10 |
| BTNL3 | DAP10 | DAP12 |
| BTNL3 | DAP10 | CD32 |
| BTNL3 | DAP10 | CD79a |
| BTNL3 | DAP10 | CD79b |
| BTNL3 | DAP12 | CD8 |
| BTNL3 | DAP12 | CD3ζ |
| BTNL3 | DAP12 | CD3δ |
| BTNL3 | DAP12 | CD3γ |
| BTNL3 | DAP12 | CD3ε |
| BTNL3 | DAP12 | FcγRI-γ |
| BTNL3 | DAP12 | FcγRIII-γ |
| BTNL3 | DAP12 | FcεRIβ |
| BTNL3 | DAP12 | FcεRIγ |
| BTNL3 | DAP12 | DAP10 |
| BTNL3 | DAP12 | DAP12 |
| BTNL3 | DAP12 | CD32 |
| BTNL3 | DAP12 | CD79a |
| BTNL3 | DAP12 | CD79b |
| BTNL3 | MyD88 | CD8 |
| BTNL3 | MyD88 | CD3ζ |
| BTNL3 | MyD88 | CD3δ |
| BTNL3 | MyD88 | CD3γ |
| BTNL3 | MyD88 | CD3ε |
| BTNL3 | MyD88 | FcγRI-γ |
| BTNL3 | MyD88 | FcγRIII-γ |
| BTNL3 | MyD88 | FcεRIβ |
| BTNL3 | MyD88 | FcεRIγ |
| BTNL3 | MyD88 | DAP10 |
| BTNL3 | MyD88 | DAP12 |
| BTNL3 | MyD88 | CD32 |
| BTNL3 | MyD88 | CD79a |
| BTNL3 | MyD88 | CD79b |
| BTNL3 | CD7 | CD8 |
| BTNL3 | CD7 | CD3ζ |
| BTNL3 | CD7 | CD3δ |
| BTNL3 | CD7 | CD3γ |
| BTNL3 | CD7 | CD3ε |
| BTNL3 | CD7 | FcγRI-γ |
| BTNL3 | CD7 | FcγRIII-γ |
| BTNL3 | CD7 | FcεRIβ |
| BTNL3 | CD7 | FcεRIγ |
| BTNL3 | CD7 | DAP10 |
| BTNL3 | CD7 | DAP12 |
| BTNL3 | CD7 | CD32 |
| BTNL3 | CD7 | CD79a |
| BTNL3 | CD7 | CD79b |
| BTNL3 | BTNL3 | CD8 |
| BTNL3 | BTNL3 | CD3ζ |
| BTNL3 | BTNL3 | CD3δ |
| BTNL3 | BTNL3 | CD3γ |
| BTNL3 | BTNL3 | CD3ε |
| BTNL3 | BTNL3 | FcγRI-γ |
| BTNL3 | BTNL3 | FcγRIII-γ |
| BTNL3 | BTNL3 | FcεRIβ |
| BTNL3 | BTNL3 | FcεRIγ |
| BTNL3 | BTNL3 | DAP10 |
| BTNL3 | BTNL3 | DAP12 |
| BTNL3 | BTNL3 | CD32 |
| BTNL3 | BTNL3 | CD79a |
| BTNL3 | BTNL3 | CD79b |
| BTNL3 | NKG2D | CD8 |
| BTNL3 | NKG2D | CD3ζ |
| BTNL3 | NKG2D | CD3δ |
| BTNL3 | NKG2D | CD3γ |
| BTNL3 | NKG2D | CD3ε |
| BTNL3 | NKG2D | FcγRI-γ |
| BTNL3 | NKG2D | FcγRIII-γ |
| BTNL3 | NKG2D | FcεRIβ |
| BTNL3 | NKG2D | FcεRIγ |
| BTNL3 | NKG2D | DAP10 |
| BTNL3 | NKG2D | DAP12 |
| BTNL3 | NKG2D | CD32 |
| BTNL3 | NKG2D | CD79a |
| BTNL3 | NKG2D | CD79b |
| NKG2D | CD28 | CD8 |
| NKG2D | CD28 | CD3ζ |
| NKG2D | CD28 | CD3δ |
| NKG2D | CD28 | CD3γ |
| NKG2D | CD28 | CD3ε |
| NKG2D | CD28 | FcγRI-γ |
| NKG2D | CD28 | FcγRIII-γ |
| NKG2D | CD28 | FcεRIβ |
| NKG2D | CD28 | FcεRIγ |
| NKG2D | CD28 | DAP10 |
| NKG2D | CD28 | DAP12 |
| NKG2D | CD28 | CD32 |
| NKG2D | CD28 | CD79a |
| NKG2D | CD28 | CD79b |
| NKG2D | CD8 | CD8 |
| NKG2D | CD8 | CD3ζ |
| NKG2D | CD8 | CD3δ |
| NKG2D | CD8 | CD3γ |
| NKG2D | CD8 | CD3ε |
| NKG2D | CD8 | FcγRI-γ |
| NKG2D | CD8 | FcγRIII-γ |
| NKG2D | CD8 | FcεRIβ |
| NKG2D | CD8 | FcεRIγ |
| NKG2D | CD8 | DAP10 |
| NKG2D | CD8 | DAP12 |
| NKG2D | CD8 | CD32 |
| NKG2D | CD8 | CD79a |
| NKG2D | CD8 | CD79b |
| NKG2D | CD4 | CD8 |
| NKG2D | CD4 | CD3ζ |
| NKG2D | CD4 | CD3δ |
| NKG2D | CD4 | CD3γ |
| NKG2D | CD4 | CD3ε |
| NKG2D | CD4 | FcγRI-γ |
| NKG2D | CD4 | FcγRIII-γ |
| NKG2D | CD4 | FcεRIβ |
| NKG2D | CD4 | FcεRIγ |
| NKG2D | CD4 | DAP10 |
| NKG2D | CD4 | DAP12 |
| NKG2D | CD4 | CD32 |
| NKG2D | CD4 | CD79a |
| NKG2D | CD4 | CD79b |
| NKG2D | b2c | CD8 |
| NKG2D | b2c | CD3ζ |
| NKG2D | b2c | CD3δ |
| NKG2D | b2c | CD3γ |
| NKG2D | b2c | CD3ε |
| NKG2D | b2c | FcγRI-γ |
| NKG2D | b2c | FcγRIII-γ |
| NKG2D | b2c | FcεRIβ |
| NKG2D | b2c | FcεRIγ |
| NKG2D | b2c | DAP10 |
| NKG2D | b2c | DAP12 |
| NKG2D | b2c | CD32 |
| NKG2D | b2c | CD79a |
| NKG2D | b2c | CD79b |
| NKG2D | CD137/41BB | CD8 |
| NKG2D | CD137/41BB | CD3ζ |
| NKG2D | CD137/41BB | CD3δ |
| NKG2D | CD137/41BB | CD3γ |
| NKG2D | CD137/41BB | CD3ε |
| NKG2D | CD137/41BB | FcγRI-γ |
| NKG2D | CD137/41BB | FcγRIII-γ |
| NKG2D | CD137/41BB | FcεRIβ |
| NKG2D | CD137/41BB | FcεRIγ |
| NKG2D | CD137/41BB | DAP10 |
| NKG2D | CD137/41BB | DAP12 |
| NKG2D | CD137/41BB | CD32 |
| NKG2D | CD137/41BB | CD79a |
| NKG2D | CD137/41BB | CD79b |
| NKG2D | ICOS | CD8 |
| NKG2D | ICOS | CD3ζ |
| NKG2D | ICOS | CD3δ |
| NKG2D | ICOS | CD3γ |
| NKG2D | ICOS | CD3ε |
| NKG2D | ICOS | FcγRI-γ |
| NKG2D | ICOS | FcγRIII-γ |
| NKG2D | ICOS | FcεRIβ |
| NKG2D | ICOS | FcεRIγ |
| NKG2D | ICOS | DAP10 |
| NKG2D | ICOS | DAP12 |
| NKG2D | ICOS | CD32 |
| NKG2D | ICOS | CD79a |
| NKG2D | ICOS | CD79b |
| NKG2D | CD27 | CD8 |
| NKG2D | CD27 | CD3ζ |
| NKG2D | CD27 | CD3δ |
| NKG2D | CD27 | CD3γ |
| NKG2D | CD27 | CD3ε |
| NKG2D | CD27 | FcγRI-γ |
| NKG2D | CD27 | FcγRIII-γ |
| NKG2D | CD27 | FcεRIβ |
| NKG2D | CD27 | FcεRIγ |
| NKG2D | CD27 | DAP10 |
| NKG2D | CD27 | DAP12 |
| NKG2D | CD27 | CD32 |
| NKG2D | CD27 | CD79a |
| NKG2D | CD27 | CD79b |
| NKG2D | CD28δ | CD8 |
| NKG2D | CD28δ | CD3ζ |
| NKG2D | CD28δ | CD3δ |
| NKG2D | CD28δ | CD3γ |
| NKG2D | CD28δ | CD3ε |
| NKG2D | CD28δ | FcγRI-γ |
| NKG2D | CD28δ | FcγRIII-γ |
| NKG2D | CD28δ | FcεRIβ |
| NKG2D | CD28δ | FcεRIγ |
| NKG2D | CD28δ | DAP10 |
| NKG2D | CD28δ | DAP12 |
| NKG2D | CD28δ | CD32 |
| NKG2D | CD28δ | CD79a |
| NKG2D | CD28δ | CD79b |
| NKG2D | CD80 | CD8 |
| NKG2D | CD80 | CD3ζ |
| NKG2D | CD80 | CD3δ |
| NKG2D | CD80 | CD3γ |
| NKG2D | CD80 | CD3ε |
| NKG2D | CD80 | FcγRI-γ |
| NKG2D | CD80 | FcγRIII-γ |
| NKG2D | CD80 | FcεRIβ |
| NKG2D | CD80 | FcεRIγ |
| NKG2D | CD80 | DAP10 |
| NKG2D | CD80 | DAP12 |
| NKG2D | CD80 | CD32 |
| NKG2D | CD80 | CD79a |
| NKG2D | CD80 | CD79b |
| NKG2D | CD86 | CD8 |
| NKG2D | CD86 | CD3ζ |
| NKG2D | CD86 | CD3δ |
| NKG2D | CD86 | CD3γ |
| NKG2D | CD86 | CD3ε |
| NKG2D | CD86 | FcγRI-γ |
| NKG2D | CD86 | FcγRIII-γ |
| NKG2D | CD86 | FcεRIβ |
| NKG2D | CD86 | FcεRIγ |
| NKG2D | CD86 | DAP10 |
| NKG2D | CD86 | DAP12 |
| NKG2D | CD86 | CD32 |
| NKG2D | CD86 | CD79a |
| NKG2D | CD86 | CD79b |
| NKG2D | OX40 | CD8 |
| NKG2D | OX40 | CD3ζ |
| NKG2D | OX40 | CD3δ |
| NKG2D | OX40 | CD3γ |
| NKG2D | OX40 | CD3ε |
| NKG2D | OX40 | FcγRI-γ |
| NKG2D | OX40 | FcγRIII-γ |
| NKG2D | OX40 | FcεRIβ |
| NKG2D | OX40 | FcεRIγ |
| NKG2D | OX40 | DAP10 |
| NKG2D | OX40 | DAP12 |
| NKG2D | OX40 | CD32 |
| NKG2D | OX40 | CD79a |
| NKG2D | OX40 | CD79b |
| NKG2D | DAP10 | CD8 |
| NKG2D | DAP10 | CD3ζ |
| NKG2D | DAP10 | CD3δ |
| NKG2D | DAP10 | CD3γ |
| NKG2D | DAP10 | CD3ε |
| NKG2D | DAP10 | FcγRI-γ |
| NKG2D | DAP10 | FcγRIII-γ |
| NKG2D | DAP10 | FcεRIβ |
| NKG2D | DAP10 | FcεRIγ |
| NKG2D | DAP10 | DAP10 |
| NKG2D | DAP10 | DAP12 |
| NKG2D | DAP10 | CD32 |
| NKG2D | DAP10 | CD79a |
| NKG2D | DAP10 | CD79b |
| NKG2D | DAP12 | CD8 |
| NKG2D | DAP12 | CD3ζ |
| NKG2D | DAP12 | CD3δ |
| NKG2D | DAP12 | CD3γ |
| NKG2D | DAP12 | CD3ε |
| NKG2D | DAP12 | FcγRI-γ |
| NKG2D | DAP12 | FcγRIII-γ |
| NKG2D | DAP12 | FcεRIβ |
| NKG2D | DAP12 | FcεRIγ |
| NKG2D | DAP12 | DAP10 |
| NKG2D | DAP12 | DAP12 |
| NKG2D | DAP12 | CD32 |
| NKG2D | DAP12 | CD79a |
| NKG2D | DAP12 | CD79b |
| NKG2D | MyD88 | CD8 |
| NKG2D | MyD88 | CD3ζ |
| NKG2D | MyD88 | CD3δ |
| NKG2D | MyD88 | CD3γ |
| NKG2D | MyD88 | CD3ε |
| NKG2D | MyD88 | FcγRI-γ |
| NKG2D | MyD88 | FcγRIII-γ |
| NKG2D | MyD88 | FcεRIβ |
| NKG2D | MyD88 | FcεRIγ |
| NKG2D | MyD88 | DAP10 |
| NKG2D | MyD88 | DAP12 |
| NKG2D | MyD88 | CD32 |
| NKG2D | MyD88 | CD79a |
| NKG2D | MyD88 | CD79b |
| NKG2D | CD7 | CD8 |
| NKG2D | CD7 | CD3ζ |
| NKG2D | CD7 | CD3δ |
| NKG2D | CD7 | CD3γ |
| NKG2D | CD7 | CD3ε |
| NKG2D | CD7 | FcγRI-γ |
| NKG2D | CD7 | FcγRIII-γ |
| NKG2D | CD7 | FcεRIβ |
| NKG2D | CD7 | FcεRIγ |
| NKG2D | CD7 | DAP10 |
| NKG2D | CD7 | DAP12 |
| NKG2D | CD7 | CD32 |
| NKG2D | CD7 | CD79a |
| NKG2D | CD7 | CD79b |
| NKG2D | BTNL3 | CD8 |
| NKG2D | BTNL3 | CD3ζ |
| NKG2D | BTNL3 | CD3δ |
| NKG2D | BTNL3 | CD3γ |
| NKG2D | BTNL3 | CD3ε |
| NKG2D | BTNL3 | FcγRI-γ |
| NKG2D | BTNL3 | FcγRIII-γ |
| NKG2D | BTNL3 | FcεRIβ |
| NKG2D | BTNL3 | FcεRIγ |
| NKG2D | BTNL3 | DAP10 |
| NKG2D | BTNL3 | DAP12 |
| NKG2D | BTNL3 | CD32 |
| NKG2D | BTNL3 | CD79a |
| NKG2D | BTNL3 | CD79b |
| NKG2D | NKG2D | CD8 |
| NKG2D | NKG2D | CD3ζ |
| NKG2D | NKG2D | CD3δ |
| NKG2D | NKG2D | CD3γ |
| NKG2D | NKG2D | CD3ε |
| NKG2D | NKG2D | FcγRI-γ |
| NKG2D | NKG2D | FcγRIII-γ |
| NKG2D | NKG2D | FcεRIβ |
| NKG2D | NKG2D | FcεRIγ |
| NKG2D | NKG2D | DAP10 |
| NKG2D | NKG2D | DAP12 |
| NKG2D | NKG2D | CD32 |
| NKG2D | NKG2D | CD79a |
| NKG2D | NKG2D | CD79b |

TABLE 4
CARs lacking Co-Simulatory Signal (for dual CAR approach)

| Co-stimulatory Signal | Signal Domain |
|---|---|
| none | CD8 |
| none | CD3ζ |
| none | CD3δ |
| none | CD3γ |
| none | CD3ε |
| none | FcγRI-γ |
| none | FcγRIII-γ |
| none | FcεRIβ |
| none | FcεRIγ |
| none | DAP10 |
| none | DAP12 |
| none | CD32 |
| none | CD79a |
| none | CD8 |
| none | CD3ζ |
| none | CD3δ |
| none | CD3γ |
| none | CD3ε |
| none | FcγRI-γ |

TABLE 5
CARs lacking Signal Domain (for dual CAR approach)

| Co-stimulatory Signal | Signal Domain |
|---|---|
| CD28 | none |
| CD8 | none |
| CD4 | none |
| b2c | none |
| CD137/41BB | none |
| ICOS | none |
| CD27 | none |
| CD28δ | none |
| CD80 | none |
| CD86 | none |
| OX40 | none |
| DAP10 | none |
| MyD88 | none |
| CD7 | none |
| DAP12 | none |
| MyD88 | none |
| CD7 | none |
| BTNL3 | none |
| NKG2D | none |

TABLE 6
Third Generation CARs lacking Signal
Domain (for dual CAR approach)

| Co-stimulatory | Co-stimulatory | Signal |
|---|---|---|
| Signal | Signal | Domain |
| CD28 | CD28 | none |
| CD28 | CD8 | none |
| CD28 | CD4 | none |
| CD28 | b2c | none |
| CD28 | CD137/41BB | none |
| CD28 | ICOS | none |
| CD28 | CD27 | none |
| CD28 | CD28δ | none |
| CD28 | CD80 | none |
| CD28 | CD86 | none |
| CD28 | OX40 | none |
| CD28 | DAP10 | none |
| CD28 | MyD88 | none |
| CD28 | CD7 | none |
| CD28 | DAP12 | none |
| CD28 | MyD88 | none |
| CD28 | CD7 | none |
| CD8 | CD28 | none |
| CD8 | CD8 | none |
| CD8 | CD4 | none |
| CD8 | b2c | none |
| CD8 | CD137/41BB | none |
| CD8 | ICOS | none |
| CD8 | CD27 | none |
| CD8 | CD28δ | none |
| CD8 | CD80 | none |
| CD8 | CD86 | none |
| CD8 | OX40 | none |
| CD8 | DAP10 | none |
| CD8 | MyD88 | none |
| CD8 | CD7 | none |
| CD8 | DAP12 | none |
| CD8 | MyD88 | none |
| CD8 | CD7 | none |
| CD4 | CD28 | none |
| CD4 | CD8 | none |
| CD4 | CD4 | none |
| CD4 | b2c | none |
| CD4 | CD137/41BB | none |
| CD4 | ICOS | none |
| CD4 | CD27 | none |
| CD4 | CD28δ | none |
| CD4 | CD80 | none |
| CD4 | CD86 | none |
| CD4 | OX40 | none |
| CD4 | DAP10 | none |
| CD4 | MyD88 | none |
| CD4 | CD7 | none |
| CD4 | DAP12 | none |
| CD4 | MyD88 | none |
| CD4 | CD7 | none |
| b2c | CD28 | none |
| b2c | CD8 | none |
| b2c | CD4 | none |
| b2c | b2c | none |
| b2c | CD137/41BB | none |
| b2c | ICOS | none |
| b2c | CD27 | none |
| b2c | CD28δ | none |
| b2c | CD80 | none |
| b2c | CD86 | none |
| b2c | OX40 | none |
| b2c | DAP10 | none |
| b2c | MyD88 | none |
| b2c | CD7 | none |
| b2c | DAP12 | none |
| b2c | MyD88 | none |
| b2c | CD7 | none |
| CD137/41BB | CD28 | none |
| CD137/41BB | CD8 | none |
| CD137/41BB | CD4 | none |
| CD137/41BB | b2c | none |
| CD137/41BB | CD137/41BB | none |
| CD137/41BB | ICOS | none |
| CD137/41BB | CD27 | none |
| CD137/41BB | CD28δ | none |
| CD137/41BB | CD80 | none |
| CD137/41BB | CD86 | none |
| CD137/41BB | OX40 | none |
| CD137/41BB | DAP10 | none |
| CD137/41BB | MyD88 | none |
| CD137/41BB | CD7 | none |
| CD137/41BB | DAP12 | none |
| CD137/41BB | MyD88 | none |
| CD137/41BB | CD7 | none |
| ICOS | CD28 | none |
| ICOS | CD8 | none |
| ICOS | CD4 | none |
| ICOS | b2c | none |
| ICOS | CD137/41BB | none |
| ICOS | ICOS | none |
| ICOS | CD27 | none |
| ICOS | CD28δ | none |
| ICOS | CD80 | none |
| ICOS | CD86 | none |
| ICOS | OX40 | none |
| ICOS | DAP10 | none |
| ICOS | MyD88 | none |
| ICOS | CD7 | none |
| ICOS | DAP12 | none |
| ICOS | MyD88 | none |
| ICOS | CD7 | none |
| ICOS | CD28 | none |
| ICOS | CD8 | none |
| ICOS | CD4 | none |
| ICOS | b2c | none |
| ICOS | CD137/41BB | none |
| ICOS | ICOS | none |
| ICOS | CD27 | none |
| ICOS | CD28δ | none |
| ICOS | CD80 | none |
| ICOS | CD86 | none |
| ICOS | OX40 | none |
| ICOS | DAP10 | none |
| ICOS | MyD88 | none |
| ICOS | CD7 | none |
| ICOS | DAP12 | none |
| ICOS | MyD88 | none |
| ICOS | CD7 | none |
| CD27 | CD28 | none |
| CD27 | CD8 | none |
| CD27 | CD4 | none |
| CD27 | b2c | none |
| CD27 | CD137/41BB | none |
| CD27 | ICOS | none |
| CD27 | CD27 | none |
| CD27 | CD28δ | none |
| CD27 | CD80 | none |
| CD27 | CD86 | none |
| CD27 | OX40 | none |
| CD27 | DAP10 | none |
| CD27 | MyD88 | none |
| CD27 | CD7 | none |
| CD27 | DAP12 | none |
| CD27 | MyD88 | none |
| CD27 | CD7 | none |
| CD28δ | CD28 | none |
| CD28δ | CD8 | none |
| CD28δ | CD4 | none |
| CD28δ | b2c | none |
| CD28δ | CD137/41BB | none |
| CD28δ | ICOS | none |
| CD28δ | CD27 | none |
| CD28δ | CD28δ | none |
| CD28δ | CD80 | none |
| CD28δ | CD86 | none |
| CD28δ | OX40 | none |
| CD28δ | DAP10 | none |
| CD28δ | MyD88 | none |
| CD28δ | CD7 | none |
| CD28δ | DAP12 | none |
| CD28δ | MyD88 | none |
| CD28δ | CD7 | none |
| CD80 | CD28 | none |
| CD80 | CD8 | none |
| CD80 | CD4 | none |
| CD80 | b2c | none |
| CD80 | CD137/41BB | none |
| CD80 | ICOS | none |
| CD80 | CD27 | none |
| CD80 | CD28δ | none |
| CD80 | CD80 | none |
| CD80 | CD86 | none |
| CD80 | OX40 | none |
| CD80 | DAP10 | none |
| CD80 | MyD88 | none |
| CD80 | CD7 | none |
| CD80 | DAP12 | none |
| CD80 | MyD88 | none |
| CD80 | CD7 | none |
| CD86 | CD28 | none |
| CD86 | CD8 | none |
| CD86 | CD4 | none |
| CD86 | b2c | none |
| CD86 | CD137/41BB | none |
| CD86 | ICOS | none |
| CD86 | CD27 | none |
| CD86 | CD28δ | none |
| CD86 | CD80 | none |
| CD86 | CD86 | none |
| CD86 | OX40 | none |
| CD86 | DAP10 | none |
| CD86 | MyD88 | none |
| CD86 | CD7 | none |
| CD86 | DAP12 | none |
| CD86 | MyD88 | none |
| CD86 | CD7 | none |
| OX40 | CD28 | none |
| OX40 | CD8 | none |
| OX40 | CD4 | none |
| OX40 | b2c | none |
| OX40 | CD137/41BB | none |
| OX40 | ICOS | none |
| OX40 | CD27 | none |
| OX40 | CD28δ | none |
| OX40 | CD80 | none |
| OX40 | CD86 | none |
| OX40 | OX40 | none |
| OX40 | DAP10 | none |
| OX40 | MyD88 | none |
| OX40 | CD7 | none |
| OX40 | DAP12 | none |
| OX40 | MyD88 | none |
| OX40 | CD7 | none |
| DAP10 | CD28 | none |
| DAP10 | CD8 | none |
| DAP10 | CD4 | none |
| DAP10 | b2c | none |
| DAP10 | CD137/41BB | none |
| DAP10 | ICOS | none |
| DAP10 | CD27 | none |
| DAP10 | CD28δ | none |
| DAP10 | CD80 | none |
| DAP10 | CD86 | none |
| DAP10 | OX40 | none |
| DAP10 | DAP10 | none |
| DAP10 | MyD88 | none |
| DAP10 | CD7 | none |
| DAP10 | DAP12 | none |
| DAP10 | MyD88 | none |
| DAP10 | CD7 | none |
| DAP12 | CD28 | none |
| DAP12 | CD8 | none |
| DAP12 | CD4 | none |
| DAP12 | b2c | none |
| DAP12 | CD137/41BB | none |
| DAP12 | ICOS | none |
| DAP12 | CD27 | none |
| DAP12 | CD28δ | none |
| DAP12 | CD80 | none |
| DAP12 | CD86 | none |
| DAP12 | OX40 | none |
| DAP12 | DAP10 | none |
| DAP12 | MyD88 | none |
| DAP12 | CD7 | none |
| DAP12 | DAP12 | none |
| DAP12 | MyD88 | none |
| DAP12 | CD7 | none |
| MyD88 | CD28 | none |
| MyD88 | CD8 | none |
| MyD88 | CD4 | none |
| MyD88 | b2c | none |
| MyD88 | CD137/41BB | none |
| MyD88 | ICOS | none |
| MyD88 | CD27 | none |
| MyD88 | CD28δ | none |
| MyD88 | CD80 | none |
| MyD88 | CD86 | none |
| MyD88 | OX40 | none |
| MyD88 | DAP10 | none |
| MyD88 | MyD88 | none |
| MyD88 | CD7 | none |
| MyD88 | DAP12 | none |
| MyD88 | MyD88 | none |
| MyD88 | CD7 | none |
| CD7 | CD28 | none |
| CD7 | CD8 | none |
| CD7 | CD4 | none |
| CD7 | b2c | none |
| CD7 | CD137/41BB | none |
| CD7 | ICOS | none |
| CD7 | CD27 | none |
| CD7 | CD28δ | none |
| CD7 | CD80 | none |
| CD7 | CD86 | none |
| CD7 | OX40 | none |
| CD7 | DAP10 | none |
| CD7 | MyD88 | none |
| CD7 | CD7 | none |
| CD7 | DAP12 | none |
| CD7 | MyD88 | none |
| CD7 | CD7 | none |
| BTNL3 | CD28 | none |
| BTNL3 | CD8 | none |
| BTNL3 | CD4 | none |
| BTNL3 | b2c | none |
| BTNL3 | CD137/41BB | none |
| BTNL3 | ICOS | none |
| BTNL3 | CD27 | none |
| BTNL3 | CD28δ | none |
| BTNL3 | CD80 | none |
| BTNL3 | CD86 | none |
| BTNL3 | OX40 | none |
| BTNL3 | DAP10 | none |
| BTNL3 | MyD88 | none |
| BTNL3 | CD7 | none |
| BTNL3 | DAP12 | none |
| BTNL3 | MyD88 | none |
| BTNL3 | CD7 | none |
| NKG2D | CD28 | none |
| NKG2D | CD8 | none |
| NKG2D | CD4 | none |
| NKG2D | b2c | none |
| NKG2D | CD137/41BB | none |
| NKG2D | ICOS | none |
| NKG2D | CD27 | none |
| NKG2D | CD28δ | none |
| NKG2D | CD80 | none |
| NKG2D | CD86 | none |
| NKG2D | OX40 | none |
| NKG2D | DAP10 | none |
| NKG2D | MyD88 | none |
| NKG2D | CD7 | none |
| NKG2D | DAP12 | none |
| NKG2D | MyD88 | none |
| NKG2D | CD7 | none |

In some embodiments, the antigen recognition domain is single chain variable fragment (scFv) antibody. The affinity/specificity of an scFv is driven in large part by specific sequences within complementarity determining regions (CDRs) in the heavy (V_{H}) and light (V_{L}) chain. Each V_{H} and V_{L} sequence will have three CDRs (CDR1, CDR2, CDR3).

In some embodiments, the antigen recognition domain is derived from natural antibodies, such as monoclonal antibodies. In some cases, the antibody is human. In some cases, the antibody has undergone an alteration to render it less immunogenic when administered to humans. For example, the alteration comprises one or more techniques selected from the group consisting of chimerization, humanization, CDR-grafting, deimmunization, and mutation of framework amino acids to correspond to the closest human germline sequence.

Nucleic Acids and Vectors

Also disclosed are polynucleotides and polynucleotide vectors encoding the disclosed CARs that allow expression of the CARs in the disclosed immune effector cells.

Nucleic acid sequences encoding the disclosed CARs, and regions thereof, can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

Expression of nucleic acids encoding CARs is typically achieved by operably linking a nucleic acid encoding the CAR polypeptide to a promoter, and incorporating the construct into an expression vector. Typical cloning vectors contain transcription and translation terminators, initiation sequences, and promoters useful for regulation of the expression of the desired nucleic acid sequence.

The disclosed nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. In some embodiments, the polynucleotide vectors are lentiviral or retroviral vectors.

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, MND (myeloproliferative sarcoma virus) promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. The promoter can alternatively be an inducible promoter. Examples of inducible promoters include, but are not limited to a metallothionine promoter, a glucocorticoid promoter, a progesterone promoter, and a tetracycline promoter.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another.

In order to assess the expression of a CAR polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene. Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York).

Biological methods for introducing a polynucleotide of interest into a host cell include the use of DNA and RNA vectors. Viral vectors, and especially retroviral vectors, have become the most widely used method for inserting genes into mammalian, e.g., human cells.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes. Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc, (Birmingham, Ala.).

Immune Effector Cells

Also disclosed are immune effector cells that are engineered to express the disclosed CARs (also referred to herein as “CAR-T cells.” These cells are preferably obtained from the subject to be treated (i.e. are autologous). However, in some embodiments, immune effector cell lines or donor effector cells (allogeneic) are used. Immune effector cells can be obtained from a number of sources, including peripheral blood mononuclear cells, bone marrow, lymph node tissue, cord blood, thymus tissue, tissue from a site of infection, ascites, pleural effusion, spleen tissue, and tumors. Immune effector cells can be obtained from blood collected from a subject using any number of techniques known to the skilled artisan, such as Ficoll™ separation. For example, cells from the circulating blood of an individual may be obtained by apheresis. In some embodiments, immune effector cells are isolated from peripheral blood lymphocytes by lysing the red blood cells and depleting the monocytes, for example, by centrifugation through a PERCOLL™ gradient or by counterflow centrifugal elutriation. A specific subpopulation of immune effector cells can be further isolated by positive or negative selection techniques. For example, immune effector cells can be isolated using a combination of antibodies directed to surface markers unique to the positively selected cells, e.g., by incubation with antibody-conjugated beads for a time period sufficient for positive selection of the desired immune effector cells. Alternatively, enrichment of immune effector cells population can be accomplished by negative selection using a combination of antibodies directed to surface markers unique to the negatively selected cells.

In some embodiments, the immune effector cells comprise any leukocyte involved in defending the body against infectious disease and foreign materials. For example, the immune effector cells can comprise lymphocytes, monocytes, macrophages, dendritic cells, mast cells, neutrophils, basophils, eosinophils, or any combinations thereof. For example, the immune effector cells can comprise T lymphocytes.

T cells or T lymphocytes can be distinguished from other lymphocytes, such as B cells and natural killer cells (NK cells), by the presence of a T-cell receptor (TCR) on the cell surface. They are called T cells because they mature in the thymus (although some also mature in the tonsils). There are several subsets of T cells, each with a distinct function.

T helper cells (T_{H} cells) assist other white blood cells in immunologic processes, including maturation of B cells into plasma cells and memory B cells, and activation of cytotoxic T cells and macrophages. These cells are also known as CD4+ T cells because they express the CD4 glycoprotein on their surface. Helper T cells become activated when they are presented with peptide antigens by MHC class II molecules, which are expressed on the surface of antigen-presenting cells (APCs). Once activated, they divide rapidly and secrete small proteins called cytokines that regulate or assist in the active immune response. These cells can differentiate into one of several subtypes, including T_{H}1, T_{H}2, T_{H}3, T_{H}17, T_{H}9, or T_{FH}, which secrete different cytokines to facilitate a different type of immune response.

Cytotoxic T cells (Tc cells, or CTLs) destroy virally infected cells and tumor cells, and are also implicated in transplant rejection. These cells are also known as CD8⁺ T cells since they express the CD8 glycoprotein at their surface. These cells recognize their targets by binding to antigen associated with MHC class I molecules, which are present on the surface of all nucleated cells. Through IL-10, adenosine and other molecules secreted by regulatory T cells, the CD8+ cells can be inactivated to an anergic state, which prevents autoimmune diseases.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory cells may be either CD4⁺ or CD8⁺. Memory T cells typically express the cell surface protein CD45RO.

Regulatory T cells (T_{reg} cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus. Two major classes of CD4⁺ T_{reg} cells have been described-naturally occurring T_{reg} cells and adaptive T_{reg} cells.

Natural killer T (NKT) cells (not to be confused with natural killer (NK) cells) bridge the adaptive immune system with the innate immune system. Unlike conventional T cells that recognize peptide antigens presented by major histocompatibility complex (MHC) molecules, NKT cells recognize glycolipid antigen presented by a molecule called CD1d.

In some embodiments, the T cells comprise a mixture of CD4+ cells. In other embodiments, the T cells are enriched for one or more subsets based on cell surface expression. For example, in some cases, the T comprise are cytotoxic CD8⁺ T lymphocytes. In some embodiments, the T cells comprise γδ T cells, which possess a distinct T-cell receptor (TCR) having one γ chain and one δ chain instead of α and β chains.

Natural-killer (NK) cells are CD56⁺CD3⁻ large granular lymphocytes that can kill virally infected and transformed cells, and constitute a critical cellular subset of the innate immune system (Godfrey J, et al. Leuk Lymphoma 2012 53:1666-1676). Unlike cytotoxic CD8⁺ T lymphocytes, NK cells launch cytotoxicity against tumor cells without the requirement for prior sensitization, and can also eradicate MHC-I-negative cells (Narni-Mancinelli E, et al. Int Immunol 2011 23:427-431). NK cells are safer effector cells, as they may avoid the potentially lethal complications of cytokine storms (Morgan R A, et al. Mol Ther 2010 18:843-851), tumor lysis syndrome (Porter D L, et al. N Engl J Med 2011 365:725-733), and on-target, off-tumor effects.

Therapeutic Methods

Immune effector cells expressing the disclosed CARs suppress alloreactive donor cells, such as T-cells, and prevent GVHD. Therefore, the disclosed CARs can be administered to any subject at risk for GVHD. In some embodiments, the subject receives a bone marrow transplant and the disclosed CAR-modified immune effector cells suppress alloreactivity of donor T-cells or dendritic cells.

The disclosed CAR-modified immune effector cells may be administered either alone, or as a pharmaceutical composition in combination with diluents and/or with other components such as IL-2, IL-15, or other cytokines or cell populations.

In some embodiments, the disclosed CAR-modified immune effector cells are administered in combination with ER stress blockade (compounds to target the IRE-1/XBP-1 pathway (e.g., B-I09). In some embodiments, the disclosed CAR-modified immune effector cells are administered in combination with a JAK2 inhibitor, a STAT3 inhibitor, an Aurora kinase inhibitor, an mTOR inhibitor, or any combination thereof.

Briefly, pharmaceutical compositions may comprise a target cell population as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions for use in the disclosed methods are in some embodiments formulated for intravenous administration. Pharmaceutical compositions may be administered in any manner appropriate treat MM. The quantity and frequency of administration will be determined by such factors as the condition of the patient, and the severity of the patient's disease, although appropriate dosages may be determined by clinical trials.

When a “therapeutic amount” is indicated, the precise amount of the compositions of the present invention to be administered can be determined by a physician with consideration of individual differences in age, weight, extent of transplantation, and condition of the patient (subject). It can generally be stated that a pharmaceutical composition comprising the T cells described herein may be administered at a dosage of 10⁴ to 10⁹ cells/kg body weight, such as 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

In certain embodiments, it may be desired to administer activated T cells to a subject and then subsequently re-draw blood (or have an apheresis performed), activate T cells therefrom according to the disclosed methods, and reinfuse the patient with these activated and expanded T cells. This process can be carried out multiple times every few weeks. In certain embodiments, T cells can be activated from blood draws of from 10 cc to 400 cc. In certain embodiments, T cells are activated from blood draws of 20 cc, 30 cc, 40 cc, 50 cc, 60 cc, 70 cc, 80 cc, 90 cc, or 100 cc. Using this multiple blood draw/multiple reinfusion protocol may serve to select out certain populations of T cells.

The administration of the disclosed compositions may be carried out in any convenient manner, including by injection, transfusion, or implantation. The compositions described herein may be administered to a patient subcutaneously, intradermally, intranodally, intramedullary, intramuscularly, by intravenous (i.v.) injection, or intraperitoneally. In some embodiments, the disclosed compositions are administered to a patient by intradermal or subcutaneous injection. In some embodiments, the disclosed compositions are administered by i.v. injection. The compositions may also be injected directly into a site of transplantation.

In certain embodiments, the disclosed CAR-modified immune effector cells are administered to a patient in conjunction with (e.g., before, simultaneously or following) any number of relevant treatment modalities, including but not limited to thalidomide, dexamethasone, bortezomib, and lenalidomide. In further embodiments, the CAR-modified immune effector cells may be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycophenolic acid, steroids, FR901228, cytokines, and irradiation. In some embodiments, the CAR-modified immune effector cells are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In another embodiment, the cell compositions of the present invention are administered following B-cell ablative therapy such as agents that react with CD20, e.g., Rituxan. For example, in some embodiments, subjects may undergo standard treatment with high dose chemotherapy followed by peripheral blood stem cell transplantation. In certain embodiments, following the transplant, subjects receive an infusion of the expanded immune cells of the present invention. In an additional embodiment, expanded cells are administered before or following surgery.

One primary concern with CAR-T cells as a form of “living therapeutic” is their manipulability in vivo and their potential immune-stimulating side effects. To better control CAR-T therapy and prevent against unwanted side effects, a variety of features have been engineered including off-switches, safety mechanisms, and conditional control mechanisms. Both self-destruct and marked/tagged CAR-T cells for example, are engineered to have an “off-switch” that promotes clearance of the CAR-expressing T-cell. A self-destruct CAR-T contains a CAR, but is also engineered to express a pro-apoptotic suicide gene or “elimination gene” inducible upon administration of an exogenous molecule. A variety of suicide genes may be employed for this purpose, including HSV-TK (herpes simplex virus thymidine kinase), Fas, iCasp9 (inducible caspase 9), CD20, MYC TAG, and truncated EGFR (endothelial growth factor receptor). HSK for example, will convert the prodrug ganciclovir (GCV) into GCV-triphosphate that incorporates itself into replicating DNA, ultimately leading to cell death. iCasp9 is a chimeric protein containing components of FK506-binding protein that binds the small molecule AP1903, leading to caspase 9 dimerization and apoptosis. A marked/tagged CAR-T cell however, is one that possesses a CAR but also is engineered to express a selection marker. Administration of a mAb against this selection marker will promote clearance of the CAR-T cell. Truncated EGFR is one such targetable antigen by the anti-EGFR mAb, and administration of cetuximab works to promotes elimination of the CAR-T cell. CARs created to have these features are also referred to as sCARs for ‘switchable CARs’, and RCARs for ‘regulatable CARs’. A “safety CAR”, also known as an “inhibitory CAR” (iCAR), is engineered to express two antigen binding domains. One of these extracellular domains is directed against a first antigen and bound to an intracellular costimulatory and stimulatory domain. The second extracellular antigen binding domain however is specific for normal tissue and bound to an intracellular checkpoint domain such as CTLA4, PD1, or CD45. Incorporation of multiple intracellular inhibitory domains to the iCAR is also possible. Some inhibitory molecules that may provide these inhibitory domains include B7-H1, B7-1, CD160, PIH, 2B4, CEACAM (CEACAM-1. CEACAM-3, and/or CEACAM-5), LAG-3, TIGIT, BTLA, LAIR1, and TGFβ-R. In the presence of normal tissue, stimulation of this second antigen binding domain will work to inhibit the CAR. It should be noted that due to this dual antigen specificity, iCARs are also a form of bi-specific CAR-T cells. The safety CAR-T engineering enhances specificity of the CAR-T cell for tissue, and is advantageous in situations where certain normal tissues may express very low levels of a antigen that would lead to off target effects with a standard CAR (Morgan 2010). A conditional CAR-T cell expresses an extracellular antigen binding domain connected to an intracellular costimulatory domain and a separate, intracellular costimulator. The costimulatory and stimulatory domain sequences are engineered in such a way that upon administration of an exogenous molecule the resultant proteins will come together intracellularly to complete the CAR circuit. In this way, CAR-T activation can be modulated, and possibly even ‘fine-tuned’ or personalized to a specific patient. Similar to a dual CAR design, the stimulatory and costimulatory domains are physically separated when inactive in the conditional CAR; for this reason these too are also referred to as a “split CAR”.

Typically, CAR-T cells are created using α-β T cells, however γ-δ T cells may also be used. In some embodiments, the described CAR constructs, domains, and engineered features used to generate CAR-T cells could similarly be employed in the generation of other types of CAR-expressing immune cells including NK (natural killer) cells, B cells, mast cells, myeloid-derived phagocytes, and NKT cells. Alternatively, a CAR-expressing cell may be created to have properties of both T-cell and NK cells. In an additional embodiment, the transduced with CARs may be autologous or allogeneic.

Several different methods for CAR expression may be used including retroviral transduction (including γ-retroviral), lentiviral transduction, transposon/transposases (Sleeping Beauty and PiggyBac systems), and messenger RNA transfer-mediated gene expression. Gene editing (gene insertion or gene deletion/disruption) has become of increasing importance with respect to the possibility for engineering CAR-T cells as well. CRISPR-Cas9, ZFN (zinc finger nuclease), and TALEN (transcription activator like effector nuclease) systems are three potential methods through which CAR-T cells may be generated.

### Definitions

The term “amino acid sequence” refers to a list of abbreviations, letters, characters or words representing amino acid residues. The amino acid abbreviations used herein are conventional one letter codes for the amino acids and are expressed as follows: A, alanine; B, asparagine or aspartic acid; C, cysteine; D aspartic acid; E, glutamate, glutamic acid; F, phenylalanine; G, glycine; H histidine; I isoleucine; K, lysine; L, leucine; M, methionine; N, asparagine; P, proline; Q, glutamine; R, arginine; S, serine; T, threonine; V, valine; W, tryptophan; Y, tyrosine; Z, glutamine or glutamic acid.

The term “antibody” refers to an immunoglobulin, derivatives thereof which maintain specific binding ability, and proteins having a binding domain which is homologous or largely homologous to an immunoglobulin binding domain. These proteins may be derived from natural sources, or partly or wholly synthetically produced. An antibody may be monoclonal or polyclonal. The antibody may be a member of any immunoglobulin class from any species, including any of the human classes: IgG, IgM, IgA, IgD, and IgE. In exemplary embodiments, antibodies used with the methods and compositions described herein are derivatives of the IgG class. In addition to intact immunoglobulin molecules, also included in the term “antibodies” are fragments or polymers of those immunoglobulin molecules, and human or humanized versions of immunoglobulin molecules that selectively bind the target antigen.

The term “antibody fragment” refers to any derivative of an antibody which is less than full-length. In exemplary embodiments, the antibody fragment retains at least a significant portion of the full-length antibody's specific binding ability. Examples of antibody fragments include, but are not limited to, Fab, Fab′, F(ab)2, scFv, Fv, dsFv diabody, Fc, and Fd fragments. The antibody fragment may be produced by any means. For instance, the antibody fragment may be enzymatically or chemically produced by fragmentation of an intact antibody, it may be recombinantly produced from a gene encoding the partial antibody sequence, or it may be wholly or partially synthetically produced. The antibody fragment may optionally be a single chain antibody fragment. Alternatively, the fragment may comprise multiple chains which are linked together, for instance, by disulfide linkages. The fragment may also optionally be a multimolecular complex. A functional antibody fragment will typically comprise at least about 50 amino acids and more typically will comprise at least about 200 amino acids.

The term “antigen binding site” refers to a region of an antibody that specifically binds an epitope on an antigen.

The term “aptamer” refers to oligonucleic acid or peptide molecules that bind to a specific target molecule. These molecules are generally selected from a random sequence pool. The selected aptamers are capable of adapting unique tertiary structures and recognizing target molecules with high affinity and specificity. A “nucleic acid aptamer” is a DNA or RNA oligonucleic acid that binds to a target molecule via its conformation, and thereby inhibits or suppresses functions of such molecule. A nucleic acid aptamer may be constituted by DNA, RNA, or a combination thereof. A “peptide aptamer” is a combinatorial protein molecule with a variable peptide sequence inserted within a constant scaffold protein. Identification of peptide aptamers is typically performed under stringent yeast dihybrid conditions, which enhances the probability for the selected peptide aptamers to be stably expressed and correctly folded in an intracellular context.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

The term “chimeric molecule” refers to a single molecule created by joining two or more molecules that exist separately in their native state. The single, chimeric molecule has the desired functionality of all of its constituent molecules. One type of chimeric molecules is a fusion protein.

The term “engineered antibody” refers to a recombinant molecule that comprises at least an antibody fragment comprising an antigen binding site derived from the variable domain of the heavy chain and/or light chain of an antibody and may optionally comprise the entire or part of the variable and/or constant domains of an antibody from any of the Ig classes (for example IgA, IgD, IgE, IgG, IgM and IgY).

The term “epitope” refers to the region of an antigen to which an antibody binds preferentially and specifically. A monoclonal antibody binds preferentially to a single specific epitope of a molecule that can be molecularly defined. In the present invention, multiple epitopes can be recognized by a multispecific antibody.

The term “fusion protein” refers to a polypeptide formed by the joining of two or more polypeptides through a peptide bond formed between the amino terminus of one polypeptide and the carboxyl terminus of another polypeptide. The fusion protein can be formed by the chemical coupling of the constituent polypeptides or it can be expressed as a single polypeptide from nucleic acid sequence encoding the single contiguous fusion protein. A single chain fusion protein is a fusion protein having a single contiguous polypeptide backbone. Fusion proteins can be prepared using conventional techniques in molecular biology to join the two genes in frame into a single nucleic acid, and then expressing the nucleic acid in an appropriate host cell under conditions in which the fusion protein is produced.

The term “Fab fragment” refers to a fragment of an antibody comprising an antigen-binding site generated by cleavage of the antibody with the enzyme papain, which cuts at the hinge region N-terminally to the inter-H-chain disulfide bond and generates two Fab fragments from one antibody molecule.

The term “F(ab′)2 fragment” refers to a fragment of an antibody containing two antigen-binding sites, generated by cleavage of the antibody molecule with the enzyme pepsin which cuts at the hinge region C-terminally to the inter-H-chain disulfide bond.

The term “Fc fragment” refers to the fragment of an antibody comprising the constant domain of its heavy chain.

The term “Fv fragment” refers to the fragment of an antibody comprising the variable domains of its heavy chain and light chain.

“Gene construct” refers to a nucleic acid, such as a vector, plasmid, viral genome or the like which includes a “coding sequence” for a polypeptide or which is otherwise transcribable to a biologically active RNA (e.g., antisense, decoy, ribozyme, etc), may be transfected into cells, e.g. in certain embodiments mammalian cells, and may cause expression of the coding sequence in cells transfected with the construct. The gene construct may include one or more regulatory elements operably linked to the coding sequence, as well as intronic sequences, polyadenylation sites, origins of replication, marker genes, etc.

The term “identity” refers to sequence identity between two nucleic acid molecules or polypeptides. Identity can be determined by comparing a position in each sequence which may be aligned for purposes of comparison. When a position in the compared sequence is occupied by the same base, then the molecules are identical at that position. A degree of similarity or identity between nucleic acid or amino acid sequences is a function of the number of identical or matching nucleotides at positions shared by the nucleic acid sequences. Various alignment algorithms and/or programs may be used to calculate the identity between two sequences, including FASTA, or BLAST which are available as a part of the GCG sequence analysis package (University of Wisconsin, Madison, Wis.), and can be used with, e.g., default setting. For example, polypeptides having at least 70%, 85%, 90%, 95%, 98% or 99% identity to specific polypeptides described herein and preferably exhibiting substantially the same functions, as well as polynucleotide encoding such polypeptides, are contemplated. Unless otherwise indicated a similarity score will be based on use of BLOSUM62. When BLASTP is used, the percent similarity is based on the BLASTP positives score and the percent sequence identity is based on the BLASTP identities score. BLASTP “Identities” shows the number and fraction of total residues in the high scoring sequence pairs which are identical; and BLASTP “Positives” shows the number and fraction of residues for which the alignment scores have positive values and which are similar to each other. Amino acid sequences having these degrees of identity or similarity or any intermediate degree of identity of similarity to the amino acid sequences disclosed herein are contemplated and encompassed by this disclosure. The polynucleotide sequences of similar polypeptides are deduced using the genetic code and may be obtained by conventional means, in particular by reverse translating its amino acid sequence using the genetic code.

The term “linker” is art-recognized and refers to a molecule or group of molecules connecting two compounds, such as two polypeptides. The linker may be comprised of a single linking molecule or may comprise a linking molecule and a spacer molecule, intended to separate the linking molecule and a compound by a specific distance.

The term “multivalent antibody” refers to an antibody or engineered antibody comprising more than one antigen recognition site. For example, a “bivalent” antibody has two antigen recognition sites, whereas a “tetravalent” antibody has four antigen recognition sites. The terms “monospecific”, “bispecific”, “trispecific”, “tetraspecific”, etc. refer to the number of different antigen recognition site specificities (as opposed to the number of antigen recognition sites) present in a multivalent antibody. For example, a “monospecific” antibody's antigen recognition sites all bind the same epitope. A “bispecific” antibody has at least one antigen recognition site that binds a first epitope and at least one antigen recognition site that binds a second epitope that is different from the first epitope. A “multivalent monospecific” antibody has multiple antigen recognition sites that all bind the same epitope. A “multivalent bispecific” antibody has multiple antigen recognition sites, some number of which bind a first epitope and some number of which bind a second epitope that is different from the first epitope.

The term “nucleic acid” refers to a natural or synthetic molecule comprising a single nucleotide or two or more nucleotides linked by a phosphate group at the 3′ position of one nucleotide to the 5′ end of another nucleotide. The nucleic acid is not limited by length, and thus the nucleic acid can include deoxyribonucleic acid (DNA) or ribonucleic acid (RNA).

The term “operably linked to” refers to the functional relationship of a nucleic acid with another nucleic acid sequence. Promoters, enhancers, transcriptional and translational stop sites, and other signal sequences are examples of nucleic acid sequences operably linked to other sequences. For example, operable linkage of DNA to a transcriptional control element refers to the physical and functional relationship between the DNA and promoter such that the transcription of such DNA is initiated from the promoter by an RNA polymerase that specifically recognizes, binds to and transcribes the DNA.

The terms “peptide,” “protein,” and “polypeptide” are used interchangeably to refer to a natural or synthetic molecule comprising two or more amino acids linked by the carboxyl group of one amino acid to the alpha amino group of another.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The terms “polypeptide fragment” or “fragment”, when used in reference to a particular polypeptide, refers to a polypeptide in which amino acid residues are deleted as compared to the reference polypeptide itself, but where the remaining amino acid sequence is usually identical to that of the reference polypeptide. Such deletions may occur at the amino-terminus or carboxy-terminus of the reference polypeptide, or alternatively both. Fragments typically are at least about 5, 6, 8 or 10 amino acids long, at least about 14 amino acids long, at least about 20, 30, 40 or 50 amino acids long, at least about 75 amino acids long, or at least about 100, 150, 200, 300, 500 or more amino acids long. A fragment can retain one or more of the biological activities of the reference polypeptide. In various embodiments, a fragment may comprise an enzymatic activity and/or an interaction site of the reference polypeptide. In another embodiment, a fragment may have immunogenic properties.

The term “protein domain” refers to a portion of a protein, portions of a protein, or an entire protein showing structural integrity; this determination may be based on amino acid composition of a portion of a protein, portions of a protein, or the entire protein.

The term “single chain variable fragment or scFv” refers to an Fv fragment in which the heavy chain domain and the light chain domain are linked. One or more scFv fragments may be linked to other antibody fragments (such as the constant domain of a heavy chain or a light chain) to form antibody constructs having one or more antigen recognition sites.

A “spacer” as used herein refers to a peptide that joins the proteins comprising a fusion protein. Generally a spacer has no specific biological activity other than to join the proteins or to preserve some minimum distance or other spatial relationship between them. However, the constituent amino acids of a spacer may be selected to influence some property of the molecule such as the folding, net charge, or hydrophobicity of the molecule.

The term “specifically binds”, as used herein, when referring to a polypeptide (including antibodies) or receptor, refers to a binding reaction which is determinative of the presence of the protein or polypeptide or receptor in a heterogeneous population of proteins and other biologics. Thus, under designated conditions (e.g. immunoassay conditions in the case of an antibody), a specified ligand or antibody “specifically binds” to its particular “target” (e.g. an antibody specifically binds to an endothelial antigen) when it does not bind in a significant amount to other proteins present in the sample or to other proteins to which the ligand or antibody may come in contact in an organism. Generally, a first molecule that “specifically binds” a second molecule has an affinity constant (Ka) greater than about 10⁵ M⁻¹ (e.g., 10⁶ M⁻¹, 10⁷ M⁻¹, 10⁸ M⁻¹, 10⁹ M⁻¹, 10¹⁰ M⁻¹, 10¹¹ M⁻¹, and 10¹² M⁻¹ or more) with that second molecule.

The term “specifically deliver” as used herein refers to the preferential association of a molecule with a cell or tissue bearing a particular target molecule or marker and not to cells or tissues lacking that target molecule. It is, of course, recognized that a certain degree of non-specific interaction may occur between a molecule and a non-target cell or tissue. Nevertheless, specific delivery, may be distinguished as mediated through specific recognition of the target molecule. Typically specific delivery results in a much stronger association between the delivered molecule and cells bearing the target molecule than between the delivered molecule and cells lacking the target molecule.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The terms “transformation” and “transfection” mean the introduction of a nucleic acid, e.g., an expression vector, into a recipient cell including introduction of a nucleic acid to the chromosomal DNA of said cell.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “variant” refers to an amino acid or peptide sequence having conservative amino acid substitutions, non-conservative amino acid substitutions (i.e. a degenerate variant), substitutions within the wobble position of each codon (i.e. DNA and RNA) encoding an amino acid, amino acids added to the C-terminus of a peptide, or a peptide having 60%, 70%, 80%, 90%, 95%, 96%, 97%, 98%, 99% sequence identity to a reference sequence.

The term “vector” refers to a nucleic acid sequence capable of transporting into a cell another nucleic acid to which the vector sequence has been linked. The term “expression vector” includes any vector, (e.g., a plasmid, cosmid or phage chromosome) containing a gene construct in a form suitable for expression by a cell (e.g., linked to a transcriptional control element).

A number of embodiments of the invention have been described.

Nevertheless, it will be understood that various modifications may be made without departing from the spirit and scope of the invention. Accordingly, other embodiments are within the scope of the following claims.

### EXAMPLES

### Example 1

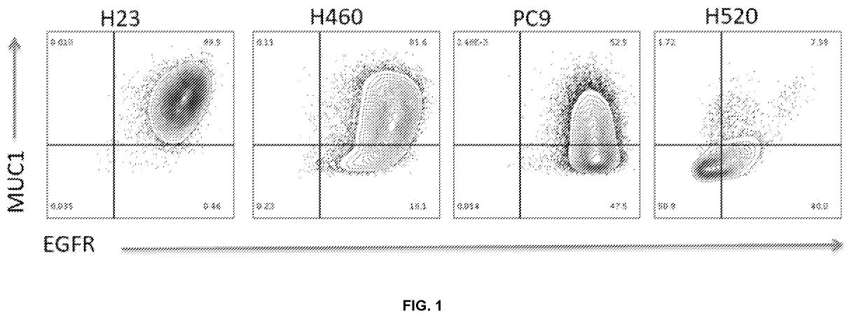

FIG. 1 shows NSCLC expresses both MUC1 and EGFR. Mucin 1 (MUC1) is a transmembrane glycoprotein that is aberrantly overexpressed in 60-80% of NSCLC cells (Sun et al, Oncology Letters 15.4 (2018): 4278-4288). The EGFR gene is overexpressed in up to 90% of NSCLC tumors (Hirsch et al, Lung Cancer. 2003; 41 Suppl 1:S29-42; Meert et al. European Respiratory Journal. 2002; 20(4):975-981). Both MUC1 and EGFR overexpression are currently being independently evaluated as targets for CART cell therapy against NSCLC (NCT 02862028, NCT 02587689).

Experiments were conducted to determine if T cells gene-targeted with CARs specific both for EGFR and MUC1 will mediate safe and effective eradication of NSCLC.

Methods

Various CAR ScFVs include Cetuximab (EGFR), C10KV3 (EGFR), SM3 (MUC1), and MUC1*. Cetuximab is an epidermal growth factor receptor binding FAB. C10KV3 are thermodynamically stable—has another kapp3 variable. The SM3 and MUC1* monoclonal antibody recognizes the under-glycosylated form of MUC1 and is therefore tumor-specific. It also reacts minimally with normal tissue.

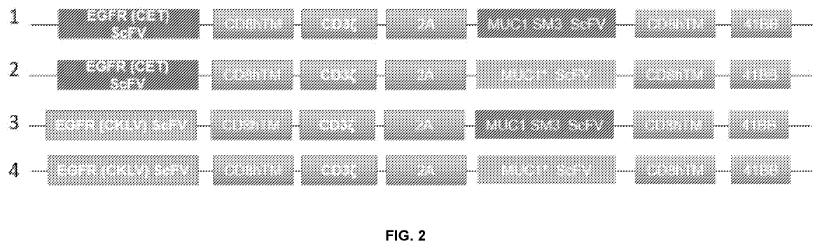

FIG. 2 shows EGFR and MUC1 CAR combinations.

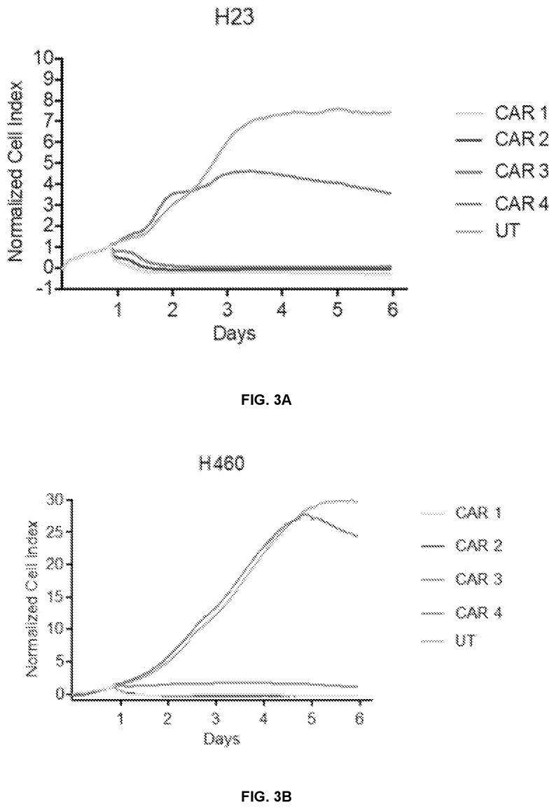

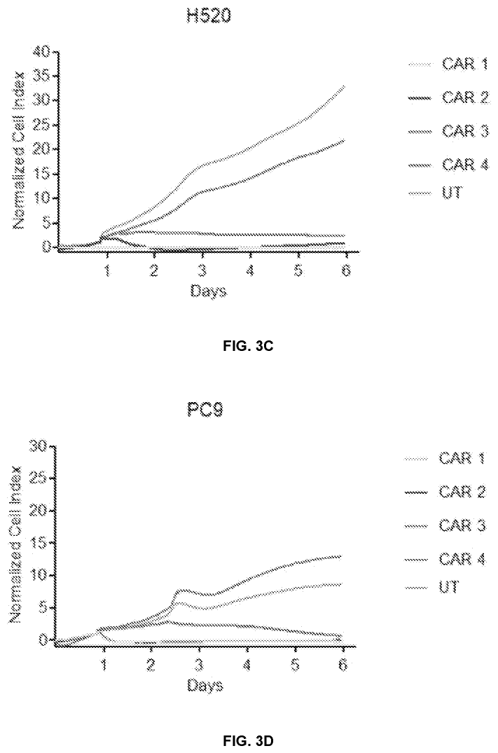

FIGS. 3A to 3D show EGFR and MUC1 bi-specific CAR-T 1, 2, and 3 elicit effector response against NSCLC.

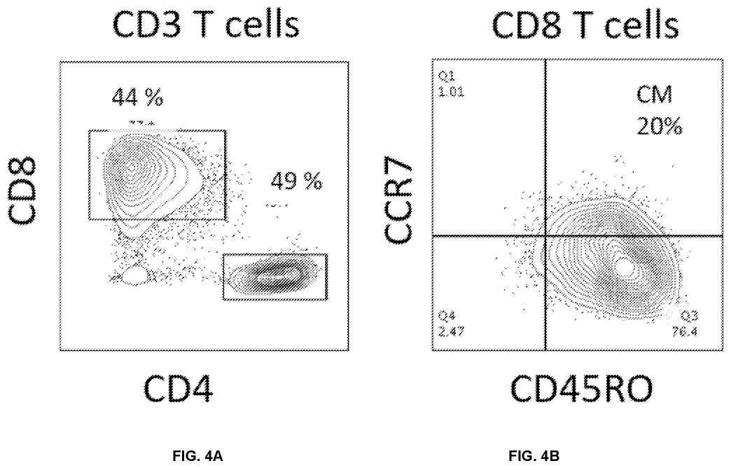

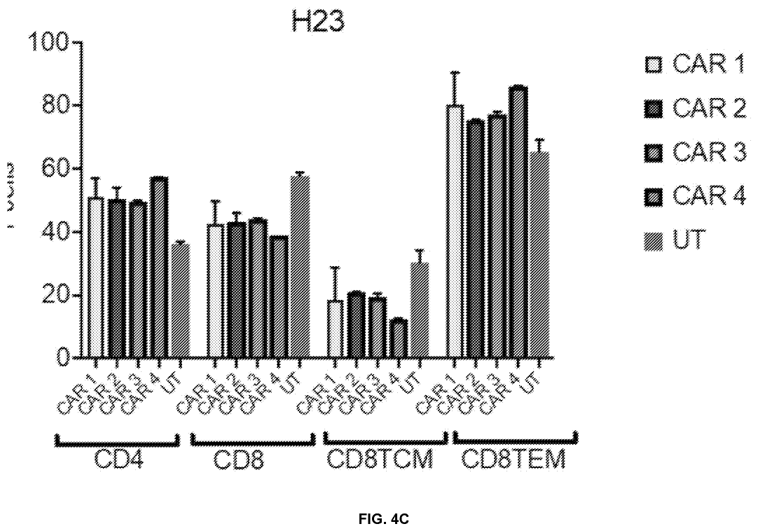

FIGS. 4A to 4C show different CARs do not show differences between different subsets of T cells.

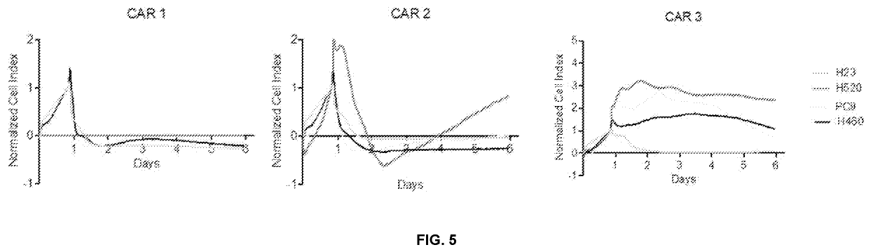

FIG. 5 shows CAR T killing comparison on different cells. Activated Bi specific EGFR and MUC1 CAR T cells or mock transduced T cells were co-cultured with target NSCLC cell lines (H23, H460, H520, and PC9) and cytotoxicity was compared via xCELLigence system as mentioned before.

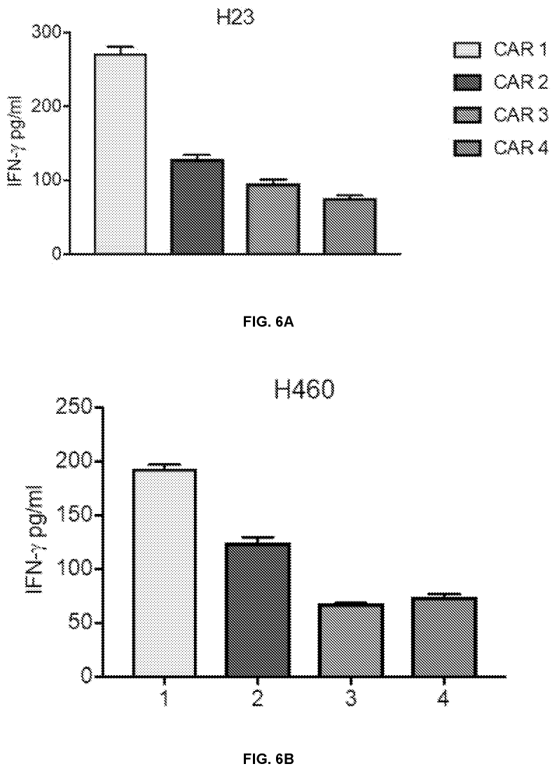

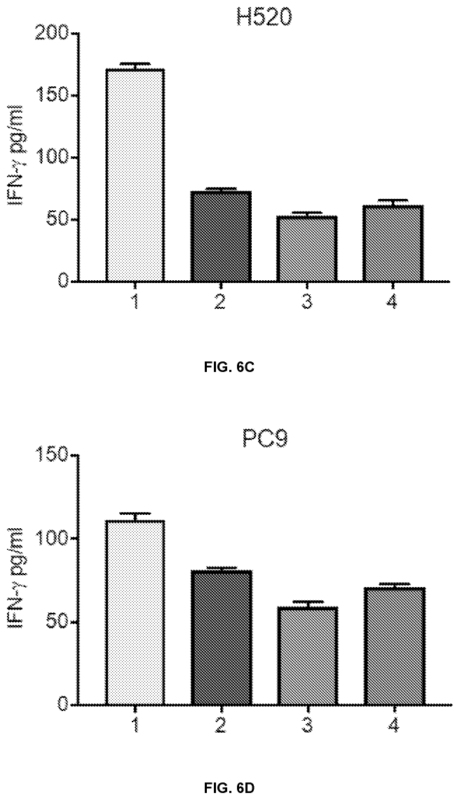

FIGS. 6A to 6D show all EGFR and MUC1 bi-specific CARs produce IFN-gamma cytokine against NSCLC cell lines. EGFR and MUC1 Bi-specific CART cell cytokine production. Activated Bi specific EGFR and MUC1 CAR T were co-cultured with indicated target cells for 24 hours. Supernatants were collected and cytokines were analyzed via Ella.

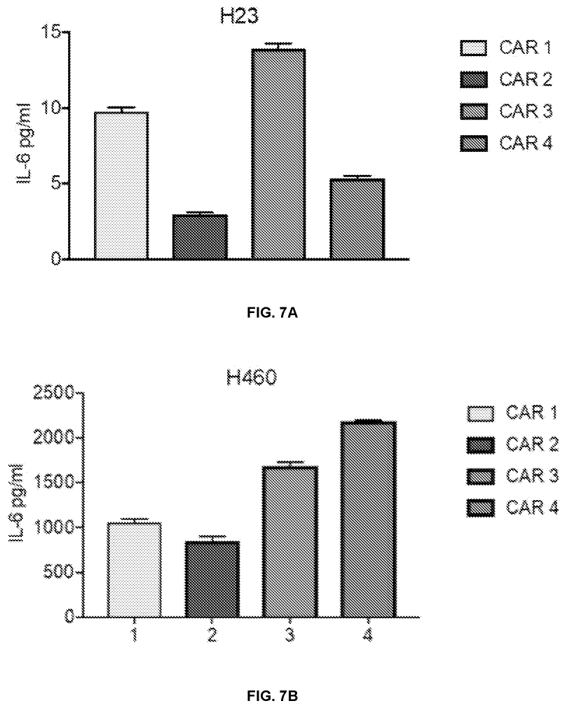

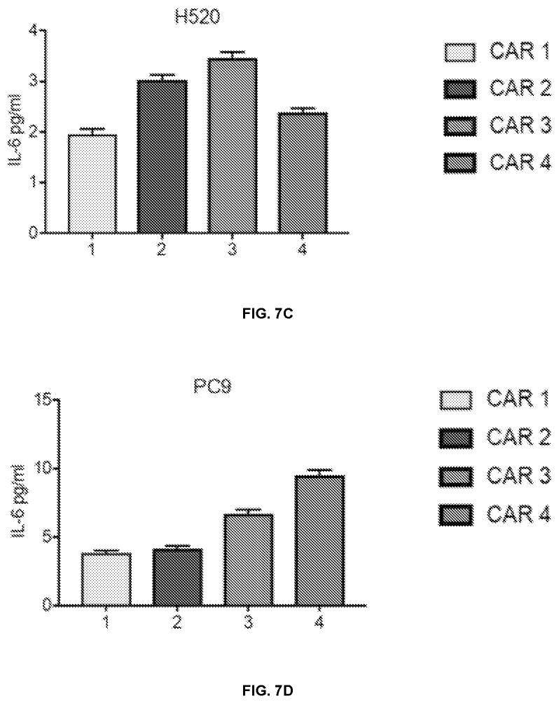

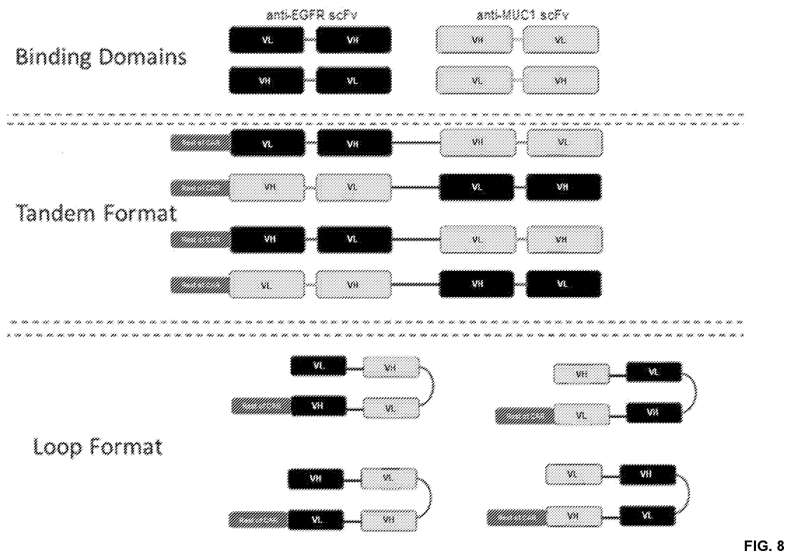

FIGS. 7A to 7D show EGFR and MUC1 Bi-specific CAR produces cytokine IL-6 against NSCLC cell lines. EGFR and MUC1 Bi-specific CART cell cytokine production. Activated Bi specific EGFR and MUC1 CAR T were co-cultured with indicated target cells for 24 hours. Supernatants were collected and IL-6 cytokine were analyzed via Ella.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. An immune effector cell engineered to express a first chimeric antigen receptor (CAR) polypeptide that selectively binds EGFR and a second chimeric antigen receptor that selectively binds MUC1.

2. The immune effector cell of claim 1, wherein the first CAR polypeptide comprises an EGFR antigen binding domain and an intracellular signaling domain, but not a co-stimulatory domain, and wherein the second CAR polypeptide comprises an MUC1 antigen binding domain and a co-stimulatory domain but not an intracellular signaling domain.

3. The immune effector cell of claim 1, wherein the first CAR polypeptide comprises an EGFR antigen binding domain and a co-stimulatory domain but not an intracellular signaling domain, and wherein the second CAR polypeptide comprises an MUC1 antigen binding domain and an intracellular signaling domain, but not a co-stimulatory domain.

4. The immune effector cell of claim 1, wherein the cell is selected from the group consisting of an αβT cell, γδT cell, a Natural Killer (NK) cells, a Natural Killer T (NKT) cell, a B cell, an innate lymphoid cell (ILC), a cytokine induced killer (CIK) cell, a cytotoxic T lymphocyte (CTL), a lymphokine activated killer (LAK) cell, a regulatory T cell, or any combination thereof.

5. The immune effector cell of claim 1, wherein the cell exhibits an anti-tumor immunity when the antigen binding domain of the first CAR polypeptide binds EGFR and the antigen binding domain of the second CAR polypeptide binds to MUC1.

6. A chimeric antigen receptor (CAR) polypeptide, comprising a EGFR antigen binding domain, a MUC1 antigen binding domain, a transmembrane domain, an intracellular signaling domain, and a co-stimulatory signaling region

7. The CAR polypeptide of claim 6, wherein the EGFR antigen binding domain is a single-chain variable fragment (scFv) of an antibody comprising a variable heavy (V_{H}) domain and a variable light (V_{L}) domain, and wherein the MUC1 antigen binding domain is a scFv comprising a V_{H} domain and a V_{L} domain.

8. The CAR polypeptide of claim 7, wherein the CAR polypeptide is defined by the formula:
SP-EV_{H}-EV_{L}-MV_{H}-MV_{L}-HG-TM-CSR/IDS;
SP-EV_{L}-EV_{H}-MV_{H}-MV_{L}-HG-TM-CSR/IDS;
SP-EV_{H}-EV_{L}-MV_{L}-M V_{H}-HG-TM-CSR/IDS;
SP-EV_{L}-EV_{H}-MV_{L}-MV_{H}-HG-TM-CSR/IDS;
SP-MV_{H}-MV_{L}-EV_{H}-EV_{L}-HG-TM-CSR/IDS;
SP-M V_{L}-MV_{H}-EV_{H}-EV_{L}-HG-TM-CSR/IDS;
SP-MV_{H}-MV_{L}-EV_{L}-EV_{H}-HG-TM-CSR/IDS; or
SP-M V_{L}-MV_{H}-EV_{L}-EV_{H}-HG-TM-CSR/IDS;
wherein “SP” represents a signal peptide,
wherein “EV_{H}” represents the EGFR scFv V_{H} domain,
wherein “EV_{L}” represents the EGFR scFv V_{L} domain,
wherein “MV_{H}” represents the MUC1 scFv V_{H} domain,
wherein “MV_{L}” represents the MUC1 scFv V_{L} domain,
wherein “HG” represents and optional hinge domain,
wherein “TM” represents a transmembrane domain,
wherein “CSR/IDS” represents a co-stimulatory signaling region and an intracellular signaling domain,
wherein “-” represents a bivalent linker.

9. The CAR polypeptide of claim 7, wherein the CAR polypeptide is defined by the formula:
SP-EV_{H}-MV_{L}-MV_{H}-EV_{L}-HG-TM-CSR/IDS;
SP-EV_{L}-MV_{L}-MV_{H}-EV_{H}-HG-TM-CSR/IDS;
SP-EV_{H}-MV_{H}-MV_{L}-EV_{L}-HG-TM-CSR/IDS;
SP-EV_{L}-MV_{H}-MV_{L}-EV_{H}-HG-TM-CSR/IDS;
SP-MV_{H}-EV_{L}-EV_{H}-MV_{L}-HG-TM-CSR/IDS;
SP-MV_{L}-EV_{L}-EV_{H}-MV_{H}-HG-TM-CSR/IDS;
SP-MV_{H}-EV_{H}-EV_{L}-MV_{L}-HG-TM-CSR/IDS;
SP-MV_{L}-EV_{H}-EV_{L}-MV_{H}-HG-TM-CSR/IDS;
wherein “SP” represents a signal peptide,
wherein “EV_{H}” represents the EGFR scFv V_{H} domain,
wherein “EV_{L}” represents the EGFR scFv V_{L} domain,
wherein “MV_{H}” represents the MUC1 scFv V_{H} domain,
wherein “MV_{L}” represents the MUC1 scFv V_{L} domain,
wherein “HG” represents and optional hinge domain,
wherein “TM” represents a transmembrane domain,
wherein “CSR/IDS” represents a co-stimulatory signaling region and an intracellular signaling domain,
wherein “-” represents a bivalent linker.

10. An immune effector cell engineered to express the CAR polypeptide of claim 7.

11. A method of providing an anti-cancer immunity in a subject with an EGFR and MUC1-expressing cancer, the method comprising administering to the subject an effective amount of the immune effector cell of claim 1, thereby providing an anti-tumor immunity in the subject.

12. The method of claim 11, further comprising administering to the subject a checkpoint inhibitor.

13. The method of claim 12, wherein the checkpoint inhibitor comprises an anti-PD-1 antibody, anti-PD-L1 antibody, anti-CTLA-4 antibody, or a combination thereof.

14. The method of claim 11, wherein the cancer comprises non-small-cell lung carcinoma (NSCLC).
